(19) United States
(12) Patent Application Publication
(10) Pub. No.: US 2006/0252744 A1
(43) Pub. Date: Nov. 9, 2006

(54) Use of N-desmethylclozapine and related compounds as dopamine stabilizing agents

(76) Inventor: Ethan S. Burstein, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/397,248

(22) Filed: Apr. 3, 2006

Related U.S. Application Data
(60) Provisional application No. 60/668,295, filed on Apr. 4, 2005.
(60) Provisional application No. 60/730,193, filed on Oct. 25, 2005.

(51) Int. Cl. A61K 31/554; A61K 31/553; A61K 31/551; A61K 31/55

(52) U.S. Cl. 514/211.13; 514/217; 514/220

(57) ABSTRACT

Disclosed herein is the use of N-desmethylclozapine (NDMC) and related compounds to treat a variety of neuropsychiatric diseases including psychosis. It is shown that NDMC and related compounds are agonists or partial agonists at D2 and D3 dopamine receptors and thus may be effective as a dopamine stabilizing agent, allowing it to be used to treat or provide reduced incidence of Extrapyramidal symptoms (EPS) and/or tardive dyskinesias (TD). Also disclosed is administering NDMC and related compounds in combination with other anti-psychotic agents.

### RELATED APPLICATIONS

This application claims the benefit of U.S. Provisional Application No. 60/668,295, filed Apr. 4, 2005 and U.S. Provisional Application No. 60/730,193 filed Oct. 25, 2005, both of which are incorporated herein by reference in their entirety.

### BACKGROUND OF THE INVENTION

1. Field of the Invention

The present invention relates to the fields of chemistry and medicine. More particularly, the present invention relates to the use of N-desmethylclozapine as a dopamine stabilizing agent and for the treatment of neuropsychiatric disease.

2. Description of the Related Art

Blockade of dopamine receptors is a key feature of antipsychotic medications and is thought to mediate many of the therapeutic effects of these drugs, particularly for the ‘positive symptoms’ of schizophrenia (1). However, antagonism of dopamine function is also responsible for many of the debilitating side effects associated with these drugs, especially the extrapyramidal side effects (EPS) and elevated serum prolactin levels (2). The antipsychotics are divided into two major classes, the typical and the atypical antipsychotics. The typical antipsychotics, exemplified by drugs such as chlorpromazine and haloperidol, were the first generation of compounds used to treat schizophrenia, and as a group tend to have uniformly higher affinity for D2 dopamine receptors, and produce a high incidence of EPS symptoms. In fact there is a strong correlation between D2 affinity, clinical dose, clinical efficacy and incidence of EPS for these agents (3, 4).

The atypical antipsychotics include many newer drugs and are distinguished by their lower incidence of EPS compared with the typical antipsychotics, while still controlling the symptoms of schizophrenia. As a group, the atypical drugs are much more heterogenous than the typical antipsychotics and thus it has been difficult to find a common mechanism of action explaining the clinical profiles of these drugs (5). The atypical drugs have varied affinities for D2 receptors, and they produce a variety of side effects including metabolic disorders, weight gain, cardiovascular effects as well as EPS in some cases. Of the atypical antipsychotics, clozapine is notable both for its beneficial effects on cognitive function (6, 7) and for its utility in treating patients that experience EPS and/or tardive dyskinesia (TD) with other antipsychotic drugs (8-10).

Dopamine hypersensitivity (also dopamine supersensitivity) caused by chronic blockade of dopamine receptors by antipsychotic drugs is a popular theory explaining the propensity of these agents to cause EPS/TD (11). Although these theories have concentrated on D2 receptor occupancy as the key determining factor, an additional consideration is that several antipsychotics are known to possess negative intrinsic activity, i.e. they are inverse agonists (12), and it is well known that inverse agonists cause recruitment and upregulation of GPCRs to the cell surface (13, 14). Therefore, D2 partial agonists may be particularly useful for treating schizophrenia because they would not be predicted to cause the upregulation of dopamine receptor tone observed with D2 inverse agonists but would still block the actions of full agonists at D2 receptors resulting in ‘dopamine stabilization’ (15, 16). In support of these ideas are the observations that aripiprazole, a newer atypical agent with partial agonist activity at D2 (17-19), has low liability for inducing EPS/TD, does not elevate serum prolactin levels, and yet is effective in controlling both the positive and negative symptoms of schizophrenia (20). In fact, it has been demonstrated that chronic treatment with aripiprazole does not upregulate either D2 binding sites or D2 mRNA whereas chronic treatment with haloperidol does (21).

These findings emphasize the importance of defining the efficacy as well as the affinity of compounds for individual receptor subtypes in order to understand their molecular basis of clinical action. There is a need for compounds that show efficacy for treating neuropsychiatric disorders, such as by having efficacy at dopamine receptors, while having a reduced incidence of EPS/TD side effects, such as by being partial agonists of dopamine receptors.

### SUMMARY OF THE INVENTION

Various aspects of the present invention include using a compound of Formula I, II, or XV: 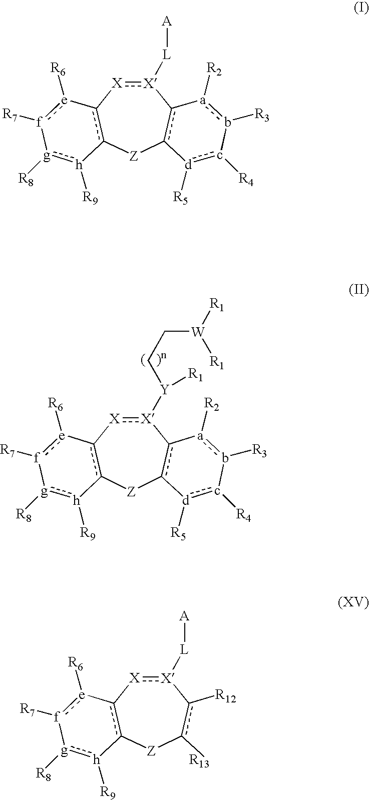
or a pharmaceutically acceptable salt, ester, amide, or prodrug thereof, wherein:
A is selected from the group consisting of 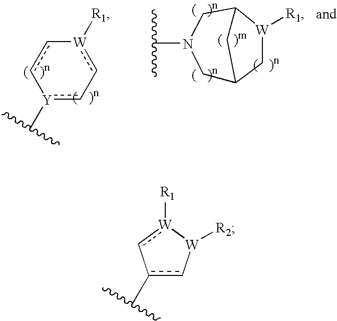
X is nitrogen, CH, or CH₂;
X′ is C or CH, wherein when X′ is C, there is a double bond between X and X′ and wherein when X′ is CH, there is a single bond between X and X′;
each Y is separately selected from the group consisting of nitrogen, oxygen, or CH;
each W is separately selected from the group consisting of nitrogen, CH, oxygen, or sulfur;
each n is separately selected from the group consisting of 0, 1, 2, 3, and 4;
m is selected from the group consisting of 1, 2, and 3;
each R₁ is separately absent or is separately selected from the group consisting of hydrogen, halogen, amine, optionally substituted C₁₋₂₀ alkyl, optionally substituted C₃₋₈ cycloalkyl, optionally substituted C₂₋₂₀ alkenyl, optionally substituted C₂₋₂₀ alkynyl, optionally substituted C₁₋₂₀-alkoxyalkyl, and optionally substituted aryl and arylalkyl;
L is absent or is selected from the group consisting of —NH(CH₂)ₙ— and —(CH₂)ₙ—;
a, b, c, and d are each separately selected from the group consisting of carbon, nitrogen, oxygen, and sulfur, or each is separately absent,
provided that at least three of a, b, c, or d are present,
provided that at least one of a, b, c, or d is carbon, and
provided that no two adjacent a, b, c, or d are both oxygen or both sulfur;
e, f, g, and h are each separately selected from the group consisting of carbon, nitrogen, oxygen, and sulfur, or each is separately absent,
provided that at least three of e, f, g, or h are present,
provided that at least one of e, f, g, or h is carbon, and
provided that no two adjacent e, f, g, or h are both oxygen or both sulfur;
R₂, R₃, R₄, and R₅, are each separately selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₂ and R₃, or R₃ and R₄, or R₄ and R₅ taken together, along with the ring carbons to which they are attached, form a five-membered or six-membered cycloalkyl, heterocyclyl or heteroaryl ring, or a six-membered aryl ring moiety;
R₆, R₇, R₈, and R₉, are each separately selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₆ and R₇, or R₇ and R₉, or R₈ and R₉ taken together, along with the ring carbons to which they are attached, form a five-membered or six-membered cycloalkyl, heterocyclyl or heteroaryl ring, or a six-membered aryl ring moiety;
Z is selected from the group consisting of NR₁₁, oxygen, sulfur, and CH₂;
R₁₀ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl optionally substituted aryl, optionally substituted arylalkyl, and perhaloalkyl; and
R₁₁ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl, and optionally substituted arylalkyl;
R₁₂ and R₁₃ are separately selected from the group consiting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₁₂ and R₁₃, taken together, along with the ring carbons to which they are attached, form a five-membered or six-membered cycloalkyl, heterocyclyl or heteroaryl ring, or a six-membered aryl ring moiety;
any bond represented by a dashed and solid line represents a bond selected from the group consisting of a carbon-carbon single bond and a carbon-carbon double bond.

In some embodiments, the compound has a structure set forth in Formulas III or IV.

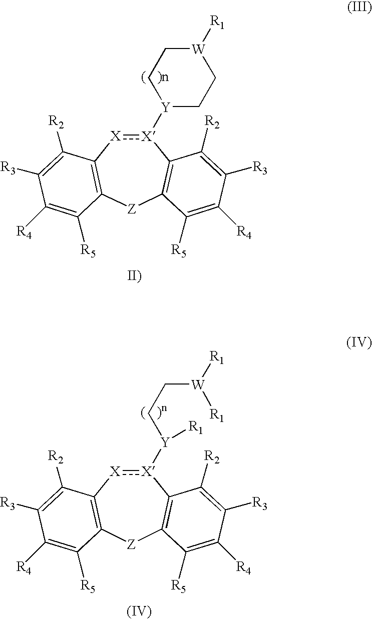

In some embodiments, the compound is selected from the group consisting of:

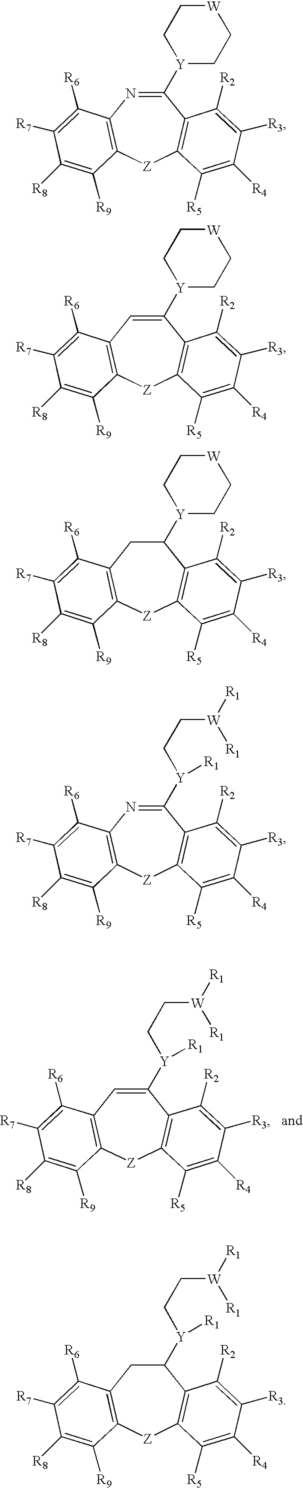

In some embodiments, the compound is selected from the group consisting of:

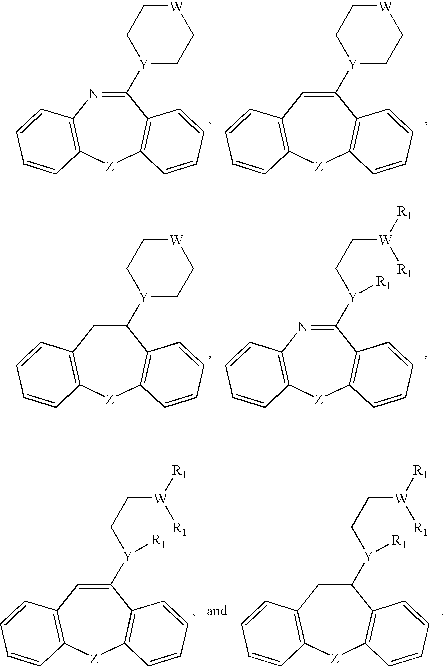

In some embodiments of the compounds described above, none of a, b, C, or d is absent. In some embodiments, none of e, f, g, or h is absent. In some embodiments, a, b, c, and d are carbon. In some embodiments, e, f, g, and h are carbon. In some embodiments, R₂ is selected from the group consisting of hydrogen, halogen, optionally substituted C₁₋₆ alkyl, and optionally substituted C₁₋₆ alkyloxy. In some embodiments, the alkyl is selected from the group consisting of methyl, ethyl, propyl, isopropyl, butyl, sec-butyl, and tert-butyl. In some embodiments, the alkyloxy is selected from the group consisting of methoxy, ethoxy, propoxy, isopropoxy, butoxy, sec-butoxy, and tert-butoxy. In some embodiments, the halogen is selected from the group consisting of fluoro, chloro, and bromo. In some embodiments, R₂ is selected from the group consisting of hydrogen, methyl, methoxy, and chloro. In some embodiments, R₃ is selected from the group consisting of hydrogen, halogen, optionally substituted C₁₋₆ alkyl, optionally substituted C₁₋₆ alkyloxy, and NO₂. In some embodiments, the alkyl is selected from the group consisting of methyl, ethyl, propyl, isopropyl, butyl, sec-butyl, and tert-butyl. In some embodiments, the alkyloxy is selected from the group consisting of methoxy, ethoxy, propoxy, isopropoxy, butoxy, sec-butoxy, and tert-butoxy. In some embodiments, the halogen is selected from the group consisting of chloro, bromo, and iodo. In some embodiments, R₃ is selected from the group consisting of hydrogen, methyl, methoxy, chloro, bromo, iodo, and NO₂. In some embodiments, R₄ is selected from the group consisting of hydrogen, halogen, optionally substituted C₁₋₆ alkyl, perhaloalkyl, SO₂R₁₀, and NO₂. In some embodiments, the alkyl is selected from the group consisting of methyl, ethyl, propyl, isopropyl, butyl, sec-butyl, and tert-butyl. In some embodiments, the perhaloalkyl is perfluoroalkyl. In some embodiments, the perfluoroalkyl is trifluoromethyl. In some embodiments, the halogen is selected from the group consisting of fluoro, chloro, and bromo. In some embodiments, R₁₀ is hydrogen or optionally substituted C₁₋₆ alkyl. In some embodiments, the alkyl is selected from the group consisting of methyl, ethyl, propyl, isopropyl, butyl, sec-butyl, and tert-butyl. In some embodiments, R₄ is selected from the group consisting of hydrogen, methyl, fluoro, chloro, bromo, trifluoromethyl, SO₂CH₃, and NO₂. In some embodiments, R₅ is selected from the group consisting of hydrogen, halogen, and optionally substituted C₁₋₆ alkyl. In some embodiments, the alkyl is selected from the group consisting of methyl, ethyl, propyl, isopropyl, butyl, sec-butyl, and tert-butyl. In some embodiments, the halogen is selected from the group consisting of fluoro, chloro, and bromo. In some embodiments, R₅ is hydrogen or chloro. In some embodiments, R₆ is hydrogen or optionally substituted C₁₋₆ alkyl. In some embodiments, R₆ is hydrogen. In some embodiments, R₇ is selected from the group consisting of hydrogen, halogen, optionally substituted C₁₋₆ alkyl, perhaloalkyl, CN, SO₂R₁₀, and NO₂. In some embodiments, the alkyl is selected from the group consisting of methyl, ethyl, propyl, isopropyl, butyl, sec-butyl, and tert-butyl. In some embodiments, the halogen is selected from the group consisting of fluoro, chloro, and bromo. In some embodiments, the perhaloalkyl is perfluoroalkyl. In some embodiments, the perfluoroalkyl is trifluoromethyl. In some embodiments, R₁₀ is hydrogen or optionally substituted C₁₋₆ alkyl. In some embodiments, the alkyl is selected from the group consisting of methyl, ethyl, propyl, isopropyl, butyl, sec-butyl, and tert-butyl. In some embodiments, R₇ is selected from the group consisting of hydrogen, methyl, chloro, trifluoromethyl, SO₂CH₃, CN, and NO₂. In some embodiments, R₈ is selected from the group consisting of hydrogen, halogen, optionally substituted C₁₋₆ alkyl. In some embodiments, the alkyl is selected from the group consisting of methyl, ethyl, propyl, isopropyl, butyl, sec-butyl, and tert-butyl. In some embodiments, the halogen is selected from the group consisting of fluoro, chloro, and bromo. In some embodiments, R₈ is selected from the group consisting of hydrogen, chloro, and bromo. In some embodiments, R₉ is selected from the group consisting of hydrogen, halogen, optionally substituted C₁₋₆ alkyl, and perhaloalkyl. In some embodiments, the alkyl is selected from the group consisting of methyl, ethyl, propyl, isopropyl, butyl, sec-butyl, and tert-butyl. In some embodiments, the halogen is selected from the group consisting of fluoro, chloro, and bromo. In some embodiments, the perhaloalkyl is perfluoroalkyl. In some embodiments, the perfluoroalkyl is trifluoromethyl. In some embodiments, R₉ is selected from the group consisting of hydrogen, chloro, methyl, and trifluoromethyl. In some embodiments, R₁ is selected from the group consisting of hydrogen, optionally substituted C₁₋₆ alkyl, and optionally substituted aryl. In some embodiments, the alkyl is selected from the group consisting of methyl, ethyl, propyl, isopropyl, butyl, sec-butyl, and tert-butyl. In some embodiments, R₁ is hydrogen. In some embodiments, X is nitrogen. In some embodiments, Y is NH. In some embodiments, L is absent or is selected from the group consisting of —NHCH₂—, —NH—, and —CH₂—. In some embodiments, A is selected from the group consisting of: 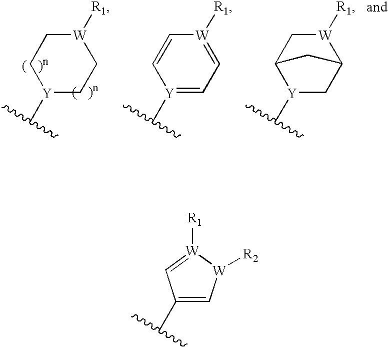
wherein n is selected from the group consiting of 0, 1, and 2.

In some embodiments, the compound is selected from the group consiting of:
2,7-Dichloro-11-(piperazin-1-yl)-5H-dibenzo[b,e][1,4]diazepine,
2-Chloro-11-(piperazin-1-yl)-5H-dibenzo[b,e][1,4]diazepine,
2,8-Dichloro-11-(piperazin-1-yl)-5H-dibenzo[b,e][1,4]diazepine,
8-Bromo-2-chloro-1-(piperazin-1-yl)-5H-dibenzo[b,e][1,4]diazepine,
2-Chloro-11-(piperazin-1-yl)-8-trifluoromethyl-5H-dibenzo[b,e][1,4]diazepine,
6-Chloro-1-(piperazin-1-yl)-8-trifluoromethyl-5H-dibenzo[b,e][1,4]diazepine,
7-Chloro-1-(piperazin-1-yl)-5H-dibenzo[b,e][1,4]diazepine,
8-Bromo-1-chloro-1-(piperazin-1-yl)-5H-dibenzo[b,e][1,4]diazepine,
8-Bromo-2-methyl-11-(piperazin-1-yl)-5H-dibenzo[b,e][1,4]diazepine,
4,8-Dichloro-11-(piperazin-1-yl)-5H-dibenzo[b,e][1,4]diazepine,
8-Chloro-2-methyl-11-(piperazin-1-yl)-5H-dibenzo[b,e][1,4]diazepine,
8-Chloro-2-fluoro-11-(piperazin-1-yl)-5H-dibenzo[b,e][1,4]diazepine,
3,8-Dichloro-11-(piperazin-1-yl)-5H-dibenzo[b,e][1,4]diazepine,
2-Bromo-8-chloro-11-(piperazin-1-yl)-5H-dibenzo[b,e][1,4]diazepine,
3,7-Dichloro-11-(piperazin-1-yl)-5H-dibenzo[b,e][1,4]diazepine,
8-Bromo-3-chloro-11-(piperazin-1-yl)-5H-dibenzo[b,e][1,4]diazepine,
3-Chloro-11-(piperazin-1-yl)-5H-dibenzo[b,e][1,4]diazepine,
3-Chloro-11-(piperazin-1-yl)-8-trifluoromethyl-5H-dibenzo[b,e][1,4]diazepine,
7-Chloro-2-methyl-11-(piperazin-1-yl)-5H-dibenzo[b,e][1,4]diazepine,
2-Methyl-11-(piperazin-1-yl)-5H-dibenzo[b,e][1,4]diazepine,
2-Methyl-11-(piperazin-1-yl)-8-trifluoromethyl-5H-dibenzo[b,e][1,4]diazepine,
8-Chloro-4-methyl-11-(piperazin-1-yl)-5H-dibenzo[b,e][1,4]diazepine,
1,8-Dichloro-11-(piperazin-1-yl)-5H-dibenzo[b,e][1,4]diazepine,
8-Bromo-5-methyl-11-(piperazin-1-yl)-5H-dibenzo[b,e][1,4]diazepine,
7,8-Dichloro-11-(piperazin-1-yl)-5H-dibenzo[b,e][1,4]diazepine,
11-(piperazin-1-yl)-8-trifluoromethyl-5H-dibenzo[b,e][1,4]diazepine,
11-(piperazin-1-yl)-5H-dibenzo[b,e][1,4]diazepine,
8-Fluoro-1-(piperazin-1-yl)-5H-dibenzo[b,e][1,4]diazepine,
11-(piperazin-1-yl)-5H-dibenzo[b,e][1,4]diazepine-8-carbonitrile,
8-Bromo-11-(piperazin-1-yl)-5H-dibenzo[b,e][1,4]diazepine,
8-Methyl-1-(piperazin-1-yl)-5H-dibenzo[b,e][1,4]diazepine,
3-Fluoro-6-piperazin-1-yl-11H-dibenzo[b,e]azepine,
2-(Trifluoromethanesulfonyloxy)-11-(piperazin-1-yl)-5H-dibenzo[b,e][1,4]diazepine,
2-(Trifluoromethanesulfonyloxy)-11-(piperazin-1-yl)-5H-dibenzo[b,e][1,4]oxazepine,
8-Chloro-2-(trifluoromethanesulfonyloxy)-11-(piperazin-1-yl)-5H-dibenzo[b,e][1,4]diazepine,
8-(Trifluoromethanesulfonyloxy)-11-(piperazin-1-yl)-5H-dibenzo[b,e][1,4]diazepine,
11-(piperazin-1-yl)-dibenzo[b,f][1,4]thiazepin,
11-(piperazin-1-yl)-2,3-dihydro-1,4-benzodioxino[6,7-b][1,4]benzothiazepin,
8-Chloro-1-[1,4]diazepam-1-yl-5H-dibenzo[b,e][1,4]diazepine,
N′-(8-Chloro-5H-dibenzo[b,e][1,4]diazepine-11-yl)-N,N-dimethyl-ethane-1,2-diamine,
N′-(8-Chloro-5H-dibenzo[b,e][1,4]diazepine-11-yl)-N,N-diethyl-ethane-1,2-diamine,
8-Chloro-11-(4-methyl-[1,4]diazepam-1-yl)-5H-dibenzo[b,e][1,4]diazepine,
8-Chloro-2-methoxy-11-(piperazin-1-yl)-5H-dibenzo[b,e][1,4]diazepine,
N′-(5H-Dibenzo[b,e][1,4]diazepine-11-yl)-N,N-dimethyl-ethane-1,2-diamine,
11-[1,4]Diazepam-1-yl-5H-dibenzo[b,e][1,4]diazepine,
N′-(8-Fluoro-5H-dibenzo[b,e][1,4]diazepine-11-yl)-N,N-dimethyl-ethane-1,2-diamine,
8-Fluoro-11-[1,4]diazepam-1-yl-5H-dibenzo[b,e][1,4]diazepine,
N′-(8-Chloro-5H-dibenzo[b,e][1,4]diazepine-1-yl)-N-methyl-ethane-1,2-diamine,
8-Chloro-11-(trans-2,5-dimethyl-piperazin-1-yl)-5H-dibenzo[b,e][1,4]diazepine,
8-Chloro-11-(3,5-dimethyl-piperazin-1-yl)-5H-dibenzo[b,e][1,4]diazepine,
8-Chloro-11-(3-methyl-piperazin-1-yl)-5H-dibenzo[b,e][1,4]diazepine,
8-Chloro-11-(3-phenyl-piperazin-1-yl)-5H-dibenzo[b,e][1,4]diazepine,
8-Chloro-5-methyl-11-(piperazin-1-yl)-5H-dibenzo[b,e][1,4]diazepine,
8-Chloro-5-benzyl-11-(piperazin-1-yl)-5H-dibenzo[b,e][1,4]diazepine,
8-Iodo-11-(piperazin-1-yl)-5H-dibenzo[b,e][1,4]diazepine,
2-Iodo-8-chloro-11-(piperazin-1-yl)-5H-dibenzo[b,e][1,4]diazepine,
8-Phenyl-11-(piperazin-1-yl)-5H-dibenzo[b,e][1,4]diazepine,
8-Chloro-11-(piperidin-1-yl)-5H-dibenzo[b,e][1,4]diazepine,
8-Chloro-11-(morpholin-4-yl)-5H-dibenzo[b,e][1,4]diazepine,
5-Allyl-8-chloro-11-(piperazin-1-yl)-5H-dibenzo[b,e][1,4]diazepine,
6-Chloro-11-(piperazin-1-yl)-5H-dibenzo[b,e][1,4]diazepine,
8-Chloro-5-piperazin-1-yl-11H-benzo[b]pyrido[2,3-e][1,4]diazepine,
2-Chloro-10-piperazin-1-yl-5H-dibenzo[b,f]azepin,
8-Chloro-11-(piperazin-1-yl)-dibenzo[b,f][1,4]thiazepine,
8-Chloro-11-(piperazin-1-yl)-dibenzo[b,f][1,4]oxazepine,
8-Chloro-11-(4-methyl-piperazin-1-yl)-dibenzo[b,f][1,4]oxazepine,
3-Chloro-6-piperazin-1-yl-1H-dibenzo[b,e]azepine,
8-Bromo-1-(piperazin-1-yl)-dibenzo[b,f][1,4]oxazepine,
11-(piperazin-1-yl)-dibenzo[b,f][1,4]oxazepine,
7-Chloro-11-(piperazin-1-yl)-dibenzo[b,f][1,4]oxazepine,
8-Chloro-3-methoxy-1-(piperazin-1-yl)-dibenzo[b,f][1,4]oxazepine,
8-Bromo-3-methoxy-1-(piperazin-1-yl)-dibenzo[b,f][1,4]oxazepine,
3-Methoxy-11-(piperazin-1-yl)-dibenzo[b,f][1,4]oxazepine,
7-Chloro-3-methoxy-11-(piperazin-1-yl)-dibenzo[b,f][1,4]oxazepine,
8-Chloro-4-methyl-11-(piperazin-1-yl)-dibenzo[b,f][1,4]oxazepine,
8-Bromo-4-methyl-11-(piperazin-1-yl)-dibenzo[b,f][1,4]oxazepine,
4-Methyl-11-(piperazin-1-yl)-dibenzo[b,f][1,4]oxazepine,
2-Bromo-8-chloro-11-(piperazin-1-yl)-dibenzo[b,f][1,4]oxazepine,
2,8-Dibromo-11-(piperazin-1-yl)-dibenzo[b,f][1,4]oxazepine,
2-Bromo-11-(piperazin-1-yl)-dibenzo[b,f][1,4]oxazepine,
2-Bromo-7-chloro-11-(piperazin-1-yl)-dibenzo[b,f][1,4]oxazepine,
11-(piperazin-1-yl)-8-trifluoromethyl-dibenzo[b,f][1,4]oxazepine,
4-Methyl-11-(piperazin-1-yl)-8-trifluoromethyl-dibenzo[b,f][1,4]oxazepine,
8-Fluoro-11-(piperazin-1-yl)-dibenzo[b,f][1,4]oxazepine,
8-Fluoro-3-methoxy-11-(piperazin-1-yl)-dibenzo[b,f][1,4]oxazepine,
8-Fluoro-4-methyl-1-(piperazin-1-yl)-dibenzo[b,f][1,4]oxazepine,
2-Bromo-8-fluoro-11-(piperazin-1-yl)-dibenzo[b,f][1,4]oxazepine,
8-Methyl-11-(piperazin-1-yl)-dibenzo[b,f][1,4]oxazepine,
3-Methoxy-8-methyl-11-(piperazin-1-yl)-dibenzo[b,f][1,4]oxazepine,
4,8-Dimethyl-11-(piperazin-1-yl)-dibenzo[b,f][1,4]oxazepine,
3-Methoxy-11-(piperazin-1-yl)-8-trifluoromethyl-dibenzo[b,f][1,4]oxazepine,
2-Bromo-11-(piperazin-1-yl)-8-trifluoromethyl-dibenzo[b,f][1,4]oxazepine,
6-Chloro-11-(piperazin-1-yl)-dibenzo[b,f][1,4]oxazepine,
2-Bromo-8-methyl-11-(piperazin-1-yl)-dibenzo[b,f][1,4]oxazepine,
7-Chloro-4-methyl-11-(piperazin-1-yl)-dibenzo[b,f][1,4]oxazepine,
8-Phenyl-11-(piperazin-1-yl)-dibenzo[b,f][1,4]oxazepine,
8-Chloro-11-(piperidin-4-yl)-5H-dibenzo[b,e][1,4]diazepine
5-Benzyl-8-chloro-11-(piperidin-4-yl)-5H-dibenzo[b,e][1,4]diazepine,
8-Bromo-5,10-dihydro-dibenzo[b,e][1,4]diazepine-11-one,
5,10-Dihydro-dibenzo[b,e][1,4]diazepine-11-one,
8-Fluoro-5,10-dihydro-dibenzo[b,e][1,4]diazepine-11-one,
8,5-Dichloro-5H-dibenzo[b,e][1,4]diazepine,
8-Chloro-11-methylsulfanyl-5H-dibenzo[b,e][1,4]diazepine
(8-Chloro-5H-dibenzo[b,e][1,4]diazepin-11-yl)-(S)-1-pyrrolidin-2-yl-methyl-amine,
1-(8-Chloro-5H-dibenzo[b,e][1,4]diazepin-11-yl)piperidine-4-yl-amine,
1-(8-Chloro-5H-dibenzo[b,e][1,4]diazepin-11-yl)-pyrrolidin-3-yl-amine,
(8-Chloro-5H-dibenzo[b,e][1,4]diazepin-11-yl)-(R)-1-pyrrolidin-2-yl-methyl-amine,
(8-Chloro-5H-dibenzo[b,e][1,4]diazepin-11-yl)-pyrrolidin-3-yl-amine,
8-Chloro-11-(2,5-diaza-bicyclo[2.2.1]hept-2-yl)-5H-dibenzo[b,e][1,4]diazepine,
Acetidin-3-yl-8-chloro-5H-dibenzo[b,e][1,4]diazepine-11-yl)amine,
7-Bromo-4-(piperazin-1-yl)-2,3-dihydro-1H-benzo[b][1,4]diazepine,
7-Bromo-2-methyl-(piperazin-1-yl)-2,3-dihydro-1H-benzo[b][1,4]diazepine
7-Bromo-2-phenyl-4-(piperazine-1-yl)-2,3-dihydro-1H-benzo[b][1,4]diazepine,
7-Bromo-10-(piperazin-1-yl)-1,2,3,3a,4,10a-hexahydro-benzo[b]cyclopenta[e][1,4]diazepine,
8-Chloro-11-(4-fluorobenzyl)-5H-dibenzo[b,e][1,4]diazepine,
8-Chloro-11-(4-fluorophenyl)-5H-dibenzo[b,e][1,4]diazepine,
8-Chloro-11-(4-nonylphenyl)-5H-dibenzo[b,e][1,4]diazepine,
8-Chloro-11-(pyridin-4-yl)-5H-dibenzo[b,e][1,4]diazepine, and
8-Chloro-11-(1H-pyrazol-4-yl)-5H-dibenzo[b,e][1,4]diazepine.

In some embodiments, the compound is N-desmethylclozapine.

One aspect of the present invention is a method of treating Extrapyramidal symptoms (EPS) and/or tardive dyskinesias (TD), comprising identifying a subject exhibiting Extrapyramidal symptoms (EPS) and/or tardive dyskinesias (TD) and administering to the subject a therapeutically effective amount of any of the compounds generically or specifically described above. In one embodiment, the subject is human.

Another aspect of the present invention is a method of ameliorating Extrapyramidal symptoms (EPS) and/or tardive dyskinesias (TD), comprising administering to a subject exhibiting Extrapyramidal symptoms (EPS) and/or tardive dyskinesias (TD) a therapeutically effective amount of N-desmethylclozapine essentially free of clozapine.

Another aspect of the present invention is a method of ameliorating Extrapyramidal symptoms (EPS) and/or tardive dyskinesias (TD), comprising administering to a subject exhibiting Extrapyramidal symptoms (EPS) and/or tardive dyskinesias (TD) a therapeutically effective amount of a pharmaceutical composition comprising N-desmethylclozapine and a pharmaceutically acceptable excipient or diluent, wherein the amount of any clozapine administered is low enough such that the combined N-desmethylclozapine and clozapine result in a net agonism at dopamine receptors.

Another aspect of the present invention is a method of treating a subject suffering from Extrapyramidal symptoms (EPS) and/or tardive dyskinesias (TD) as a result of exposure to one or more medications, comprising identifying a subject exhibiting Extrapyramidal symptoms (EPS) and/or tardive dyskinesias (TD) as a result of exposure to one or more medications and administering to the subject a therapeutically effective amount of any of the compounds generically or specifically described above. In one embodiment, the subject is human.

Another aspect of the present invention is a method of treating a subject refractory to other treatments due to a propensity to develop Extrapyramidal symptoms (EPS) and/or tardive dyskinesias (TD), comprising administering to a subject having a propensity to develop Extrapyramidal symptoms (EPS) and/or tardive dyskinesias (TD) a therapeutically effective amount of any of the compounds generically or specifically described above. One embodiment further comprises identifying a subject having a propensity to develop Extrapyramidal symptoms (EPS) and/or tardive dyskinesias (TD).

Another aspect of the present invention is a method of treating a subject refractory to other treatments due to a propensity to develop Extrapyramidal symptoms (EPS) and/or tardive dyskinesias (TD), comprising administering to a subject having a propensity to develop Extrapyramidal symptoms (EPS) and/or tardive dyskinesias (TD) a therapeutically effective amount of N-desmethylclozapine essentially free of clozapine. One embodiment further comprises identifying a subject having a propensity to develop Extrapyramidal symptoms (EPS) and/or tardive dyskinesias (TD).

Another aspect of the present invention is a method of treating a subject refractory to other treatments due to a propensity to develop Extrapyramidal symptoms (EPS) and/or tardive dyskinesias (TD), comprising administering to the subject having a propensity to develop Extrapyramidal symptoms (EPS) and/or tardive dyskinesias (TD) a therapeutically effective amount of a pharmaceutical composition comprising N-desmethylclozapine and a pharmaceutically acceptable excipient or diluent, wherein the amount of any clozapine administered is low enough such that the combined N-desmethylclozapine and clozapine result in a net agonism at dopamine receptors.

Another aspect of the present invention is a method of dopamine stabilization, comprising identifying a subject in need of dopamine stabilization and administering to the subject an amount of any of the compounds generically or specifically described above effective to stabilize one or more dopamine receptors. In one embodiment, the dopamine receptor is a D2 receptor.

Another aspect of the present invention is a method of treating psychosis, comprising administering to a subject any of the compounds generically or specifically described above in combination with another anti-psychotic agent. In one embodiment, the dosage of the other anti-psychotic agent administered is less than the dosage that would be typically used if the other anti-psychotic agent were administered alone. In one embodiment, the other anti-psychotic agent is selected from the group consisting of a phenothiazine, phenylbutylpiperadine, debenzapine, benzisoxidil, and a salt of lithium. In one embodiment, the phenothiazine is selected from the group consisting of chlorpromazine (Thorazine®), mesoridazine (Serentil®), prochlorperazine (Compazine®), and thioridazine (Mellaril®). In one embodiment, the phenylbutylpiperadine is selected from the group consisting of haloperidol (Haldol®) and pimozide (Orap®). In one embodiment, the debenzapine is selected from the group consisting of clozapine (Clozaril®), loxapine (Loxitane®), olanzapine (Zyprexa®) and quetiapine (Seroquel®). In one embodiment, the benzisoxidil is selected from the group consisting of resperidone (Resperidal®) and ziprasidone (Geodon®). In one embodiment, the salt of lithium is lithium carbonate. In one embodiment, the antipsychotic agent is selected from the group consisting of Aripiprazole (Abilify), Clozapine, Clozaril, Compazine, Etrafon, Geodon, Haldol, Inapsine, Loxitane, Mellaril, Moban, Navane, Olanzapine (Zyprexa), Orap, Permitil, Prolixin, Phenergan, Quetiapine (Seroquel), Reglan, Risperdal, Serentil, Seroquel, Stelazine, Taractan, Thorazine, Triavil, Trilafon, and Zyprexa, or pharmaceutically acceptable salts thereof.

Another aspect of the present invention is a pharmaceutical composition comprising any of the compounds generically or specifically described above and another anti-psychotic agent. In one embodiment, the dosage of the other anti-psychotic agent in the pharmaceutical composition is less than the dosage that would be typically used if the other anti-psychotic agent were administered alone. In one embodiment, the other anti-psychotic agent is selected from the group consisting of a phenothiazine, phenylbutylpiperadine, debenzapine, benzisoxidil, and a salt of lithium. In one embodiment, the phenothiazine is selected from the group consisting of chlorpromazine (Thorazine®), mesoridazine (Serentil®), prochlorperazine (Compazine®), and thioridazine (Mellaril®). In one embodiment, the phenylbutylpiperadine is selected from the group consisting of haloperidol (Haldol®) and pimozide (Orap®). In one embodiment, the debenzapine is selected from the group consisting of clozapine (Clozaril®), loxapine (Loxitane®), olanzapine (Zyprexa®) and quetiapine (Seroquel®). In one embodiment, the benzisoxidil is selected from the group consisting of resperidone (Resperidal®) and ziprasidone (Geodon®). In one embodiment, the salt of lithium is lithium carbonate. In one embodiment, the antipsychotic agent is selected from the group consisting of Aripiprazole (Abilify), Clozapine, Clozaril, Compazine, Etrafon, Geodon, Haldol, Inapsine, Loxitane, Mellaril, Moban, Navane, Olanzapine (Zyprexa), Orap, Permitil, Prolixin, Phenergan, Quetiapine (Seroquel), Reglan, Risperdal, Serentil, Seroquel, Stelazine, Taractan, Thorazine, Triavil, Trilafon, and Zyprexa, or pharmaceutically acceptable salts thereof. In one embodiment, the pharmaceutical composition is essentially free of clozapine. In one embodiment, the amount of any clozapine in the composition is low enough such that the combined N-desmethylclozapine and clozapine administered to a subject when the composition is administered to the subject result in a net agonism at dopamine receptors.

Another aspect of the present invention is a method of modulating D2 receptors, comprising identifying a subject in need of D2 receptor modulation and contacting D2 receptors in the subject with any of the compounds generically or specifically described above.

Another aspect of the present invention is a method of modulating D2 receptors, comprising identifying a subject in need of D2 receptor modulation and contacting D2 receptors in the subject with N-desmethylclozapine, wherein any clozapine also contacting the D2 receptors is low enough such that the combined N-desmethylclozapine and clozapine contacting the D2 receptors result in a net agonism of the D2 receptors.

Another aspect of the present invention is a method of modulating D3 receptors, comprising identifying a subject in need of D3 receptor modulation and contacting D3 receptors in the subject with any of the compounds generically or specifically described above.

Another aspect of the present invention is a method of modulating D3 receptors, comprising identifying a subject in need of D3 receptor modulation and contacting D3 receptors in the subject with N-desmethylclozapine, wherein any clozapine also contacting the D3 receptors is low enough such that the combined N-desmethylclozapine and clozapine contacting the D3 receptors result in a net agonism of the D3 receptors.

Another aspect of the present invention is a method of ameliorating one or more symptoms of a condition associated with a dopamine receptor, comprising identifying a subject exhibiting the one or more symptoms and administering to the subject a therapeutically effective amount of any of the compounds generically or specifically described above.

Another aspect of the present invention is a method of ameliorating one or more symptoms of a condition associated with a dopamine receptor, comprising identifying a subject exhibiting the one or more symptoms and administering to the subject a therapeutically effective amount of a pharmaceutical composition comprising N-desmethylclozapine and a pharmaceutically acceptable excipient or diluent, wherein the amount of any clozapine administered is low enough such that the combined N-desmethylclozapine and clozapine result in a net agonism at the dopamine receptor.

### BRIEF DESCRIPTION OF THE DRAWINGS

FIGS. 1A and 1B depict bar graphs illustrating the activity of various anti-psychotic agents at dopamine D2 (FIG. 1A) and D3 (FIG. 1B) receptors.

FIGS. 2A and 2B depict activity-concentration curves of N-desmethylclozapine, haloperidol, pergolide, and clozapine at dopamine D3 (FIG. 2A) and D2 (FIG. 2B) receptors.

FIGS. 3A and 3B depict activity-concentration curves of N-desmethylclozapine (NDMC), clozapine+NDMC, and haloperidol+NDMC at dopamine D3 (FIG. 3A) and D2 (FIG. 3B) receptors.

### DETAILED DESCRIPTION OF THE PREFERRED EMBODIMENT

A large series of drugs that have utility in treating schizophrenia were profiled for intrinsic efficacy at the human D2 and D3 dopamine receptors. All of the antipsychotics tested were inverse agonists at the D2 and D3 dopamine receptors with the exception of only two agents; the atypical antipsychotic aripiprazole and the primary active metabolite of clozapine, N-desmethylclozapine.

The administration of clozapine to human subjects results in the formation of two major metabolites: N-desmethylclozapine (NDMC) and clozapine-N-oxide (22). However, clozapine-N-oxide is a polar metabolite that is rapidly excreted and likely does not contribute to the biological activity of the parent compound. A correlation exists between the dose of clozapine administered to a subject, and the serum levels of total clozapine moieties, yet the levels of NDMC can vary widely between individual subjects (23). Generally, NDMC constitutes 40-75% of the total serum clozapine concentrations during steady state kinetics in humans (24). Conflicting data exists as to the ability of NDMC to penetrate the blood brain barrier and impart centrally mediated activity (25, 26). These observations demonstrate that NDMC in the serum of human subjects is well tolerated. Few data exist as to the molecular properties of NDMC. NDMC has been shown to possess antagonist activity at 5HT_{2C} receptors (27). Furthermore, NDMC has be shown to be active at muscarinic receptors as described in U.S. application Ser. Nos. 10/761,787 and 10/913,117, both of which are incorporated herein by reference in their entirety. However, no data on its functional interaction with dopaminergic receptors has been reported.

Surprisingly, and unlike the closely related compound clozapine, it has been found that the compound N-desmethylclozapine (NDMC) and related analogs possesses heretofore unappreciated functional activity as a D2 and D3 receptor agonist. The molecular activities of NDMC and related analogs, as identified by the methods described herein, combined with the known clinical efficacy of compounds that possess a similar molecular pharmacological profile, indicate that NDMC and its analogs can be used to alleviate or treat disorders or conditions associated with human psychosis including treatment-induced psychosis in Parkinson's patients, patients suffering from extra-pyramidal symptoms (EPS) or tardive dyskinesia (TD), patients refractory to treatment with other antipsychotic medications due to dose-limiting side effects such as EPS or TD, mania, affective disease, degenerative dementia, glaucoma, and neuropathic pain.

Thus, in one embodiment, the present invention relates to the use of compounds of Formula I, II, or XV or a pharmaceutically acceptable salt, ester, amide, or prodrug thereof in human subjects to ameliorate one or more symptoms associated with schizophrenia or psychosis of any origin: 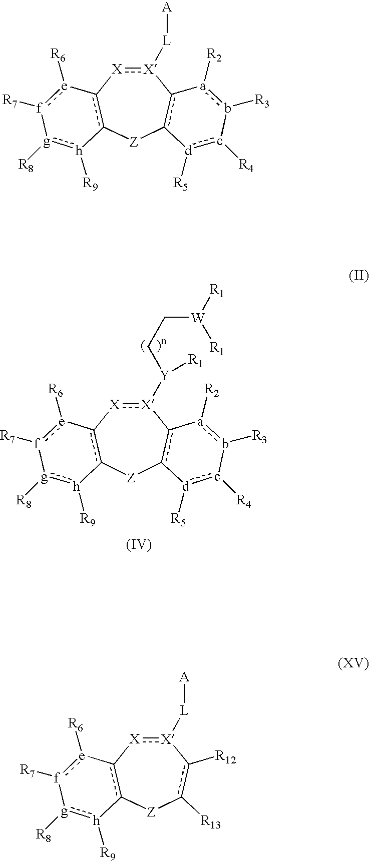
wherein:

A is selected from the group consisting of

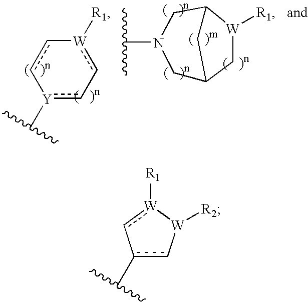

X is nitrogen, CH, or CH₂;

X′ is C or CH, wherein when X′ is C, there is a double bond between X and X′ and wherein when X′ is CH, there is a single bond between X and X′;

each Y is separately selected from the group consisting of nitrogen, oxygen, or CH;

each W is separately selected from the group consisting of nitrogen, CH, oxygen, or sulfur;

each n is separately selected from the group consisting of 0, 1, 2, 3, and 4;

m is selected from the group consisting of 1, 2, and 3;

each R₁ is separately absent or is separately selected from the group consisting of hydrogen, halogen, amine, optionally substituted C₁₋₂₀ alkyl, optionally substituted C₃₋₈ cycloalkyl, optionally substituted C₂₋₂₀ alkenyl, optionally substituted C₂₋₂₀ alkynyl, optionally substituted C₁₋₂₀-alkoxyalkyl, and optionally substituted aryl and arylalkyl;

L is absent or is selected from the group consisting of —(NH(CH₂)ₙ— and —(CH₂)ₙ—;

a, b, c, and d are each independently selected from the group consisting of carbon, nitrogen, oxygen, and sulfur, or each is independently absent,
provided that at least three of a, b, c, or d are present,
provided that at least one of a, b, c, or d is carbon, and
provided that no two adjacent a, b, c, or d are both oxygen or both sulfur;

e, f, g, and h are each independently selected from the group consisting of carbon, nitrogen, oxygen, and sulfur, or each is independently absent,
provided that at least three of e, f, g, or h are present,
provided that at least one of e, f, g, or h is carbon, and
provided that no two adjacent e, f, g, or h are both oxygen or both sulfur;

R₂, R₃, R₄, and R₅, are each independently selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,

or R₂ and R₃, or R₃ and R₄, or R₄ and R₅ taken together, along with the ring carbons to which they are attached, form a five-membered or six-membered cycloalkyl, heterocyclyl or heteroaryl ring, or a six-membered aryl ring moiety;

R₆, R₇, R₈, and R₉, are each independently selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,

or R₆ and R₇, or R₇ and R₈, or R₈ and R₉ taken together, along with the ring carbons to which they are attached, form a five-membered or six-membered cycloalkyl, heterocyclyl or heteroaryl ring, or a six-membered aryl ring moiety;

Z is selected from the group consisting of NR₁₁, oxygen, sulfur, and CH₂;

R₁₀ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl optionally substituted aryl, optionally substituted arylalkyl, and perhaloalkyl;

R₁₁ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl, and optionally substituted arylalkyl;

R₁₂ and R₁₃ are separately selected from the group consiting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,

or R₁₂ and R₁₃, taken together, along with the ring carbons to which they are attached, form a five-membered or six-membered cycloalkyl, heterocyclyl or heteroaryl ring, or a six-membered aryl ring moiety.

Bonds represented by a dashed and solid line represents a bond selected from the group consisting of a carbon-carbon single bond and a carbon-carbon double bond. The dashed bond between X and X′ in Formulae I, II, and XV indicates that X and X′ may be joined by either a single or a double bond.

In certain embodiments, the compound of Formulae I and XV does not include clozapine, the structure of which is shown below:

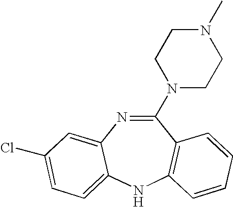

In certain embodiments, in compounds of Formulae I and XV, Y is nitrogen or CH. In other embodiments, in compounds of Formula II, Y is nitrogen, oxygen or CH.

In certain embodiments, the compounds of Formula I or XV are selected from the following structures: 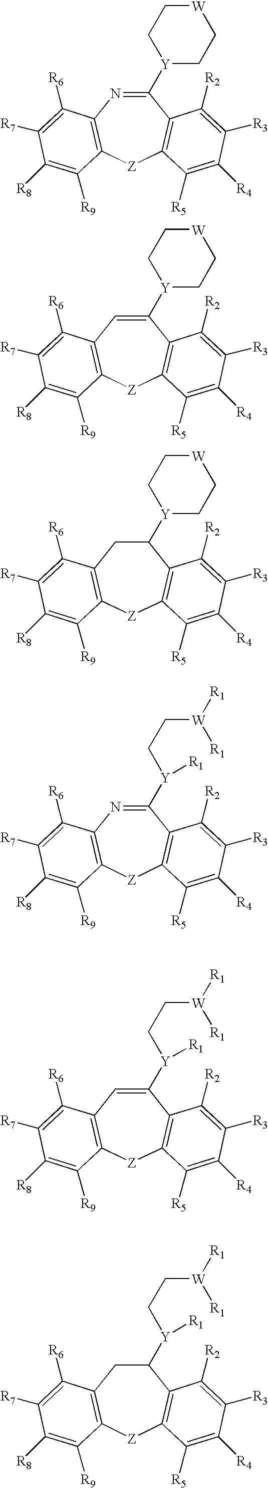
where R₁-R₉, W, Y, and Z are as described herein.

In certain other embodiments, the compounds of Formula I or XV are selected from the following structures: 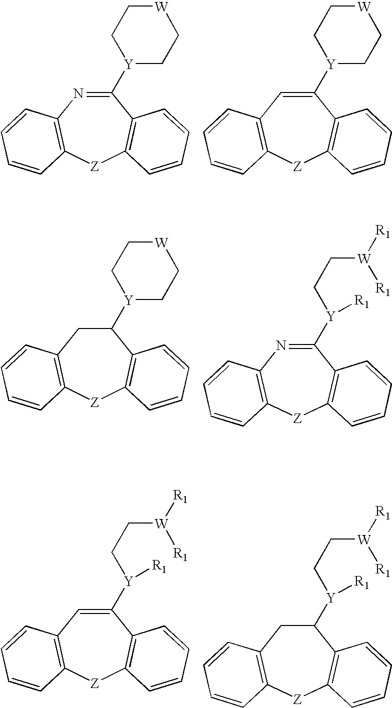
where R₁, W, Y, and Z are as described herein.

In certain embodiments, the compounds of Formula I or XV are selected from the structure set forth in Formula III or Formula IV. 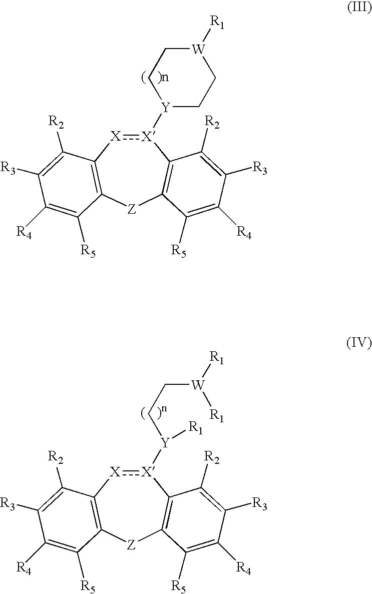
where R₁-R₅, W, X, X′, Y, and Z are as described herein.

In certain embodiments, none of a, b, c, or d is absent, and the ring formed thereby is a six-membered ring. In further embodiments, none of e, f, g, or h is absent, and consequently, the ring formed thereby is a six-membered ring. In some embodiments, a, b, c, and d are carbon, and the ring formed thereby is an optionally substituted phenyl ring. In further embodiments, e, f, g, and h are carbon, which similarly form an optionally substituted phenyl ring.

In certain embodiments, R₂ may be selected from the group consisting of hydrogen, halogen, optionally substituted C₁₋₆ alkyl, and optionally substituted C₁₋₆ alkyloxy. In some embodiments, the alkyl may be selected from the group consisting of methyl, ethyl, propyl, isopropyl, butyl, sec-butyl, and tert-butyl. In other embodiments, the alkyloxy may be selected from the group consisting of methoxy, ethoxy, propoxy, isopropoxy, butoxy, sec-butoxy, and tert-butoxy. In further embodiments, the halogen may be selected from the group consisting of fluoro, chloro, and bromo. In certain embodiments, R₂ may be selected from the group consisting of hydrogen, methyl, methoxy, and chloro.

In some embodiments, R₃ may be selected from the group consisting of hydrogen, halogen, optionally substituted C₁₋₆ alkyl, optionally substituted C₁₋₆ alkyloxy, and NO₂. The alkyl group may be selected from the group consisting of methyl, ethyl, propyl, isopropyl, butyl, sec-butyl, and tert-butyl, while the alkoxy may be selected from the group consisting of methoxy, ethoxy, propoxy, isopropoxy, butoxy, sec-butoxy, and tert-butoxy. In further embodiments, the halogen may be selected from the group consisting of chloro, bromo, and iodo. In other embodiments, R₃ may be selected from the group consisting of hydrogen, methyl, methoxy, chloro, bromo, iodo, and NO₂.

In certain embodiments, R₄ may be selected from the group consisting of hydrogen, halogen, optionally substituted C₁₋₆ alkyl, perhaloalkyl, SO₂R₁₀, and NO₂. In some embodiments, the alkyl may be selected from the group consisting of methyl, ethyl, propyl, isopropyl, butyl, sec-butyl, and tert-butyl. In further embodimetns, the perhaloalkyl may be perfluoroalkyl, which in some embodiments, may be trifluoromethyl. In other embodiments, the halogen may be selected from the group consisting of fluoro, chloro, and bromo. When R₄ is SO₂R₁₀, the R₁₀ may be hydrogen or optionally substituted C₁₋₆ alkyl, which alkyl may be selected from the group consisting of methyl, ethyl, propyl, isopropyl, butyl, sec-butyl, and tert-butyl. In certain embodiments, R₄ may be selected from the group consisting of hydrogen, methyl, fluoro, chloro, bromo, trifluoromethyl, SO₂CH₃, and NO₂.

In some embodiments, R₅ may be selected from the group consisting of hydrogen, halogen, and optionally substituted C₁₋₆ alkyl. The alkyl may be selected from the group consisting of methyl, ethyl, propyl, isopropyl, butyl, sec-butyl, and tert-butyl, while the halogen may be selected from the group consisting of fluoro, chloro, and bromo. In certain embodiments, R₅ may be hydrogen or chloro.

In certain embodiments, R₆ may be hydrogen or optionally substituted C₁₋₆ alkyl. The alkyl may be selected from the group consisting of methyl, ethyl, propyl, isopropyl, butyl, sec-butyl, and tert-butyl. In some embodiments, R₆ may be hydrogen.

In certain embodiments, R₇ may be selected from the group consisting of hydrogen, halogen, optionally substituted C₁₋₆ alkyl, perhaloalkyl, CN, SO₂R₁₀, and NO₂. The alkyl may be selected from the group consisting of methyl, ethyl, propyl, isopropyl, butyl, sec-butyl, and tert-butyl, while the halogen may be selected from the group consisting of fluoro, chloro, and bromo. In some embodiments, the perhaloalkyl is perfluoroalkyl, which in some embodiments, may be trifluoromethyl. In the embodiments in which R₇ may be SO₂R₁₀, R₁₀ may be hydrogen or optionally substituted C₁₋₆ alkyl, which alkyl may be selected from the group consisting of methyl, ethyl, propyl, isopropyl, butyl, sec-butyl, and tert-butyl. In certain embodiments, R₇ may be selected from the group consisting of hydrogen, methyl, chloro, trifluoromethyl, SO₂CH₃, CN, and NO₂.

In some embodiments, R₈ may be selected from the group consisting of hydrogen, halogen, optionally substituted C₁₋₆ alkyl, which alkyl may be selected from the group consisting of methyl, ethyl, propyl, isopropyl, butyl, sec-butyl, and tert-butyl. The halogen may be selected from the group consisting of fluoro, chloro, and bromo. In certain embodiments, R₈ may be selected from the group consisting of hydrogen, chloro, and bromo.

Embodiments of the present disclosure include those in which R₉ may be selected from the group consisting of hydrogen, halogen, optionally substituted C₁₋₆ alkyl, and perhaloalkyl. The alkyl may be selected from the group consisting of methyl, ethyl, propyl, isopropyl, butyl, sec-butyl, and tert-butyl. The halogen may be selected from the group consisting of fluoro, chloro, and bromo. The perhaloalkyl may be perfluoroalkyl, which in some embodiments may be trifluoromethyl. In some embodiments, R₉ may be selected from the group consisting of hydrogen, chloro, methyl, and trifluoromethyl.

In some embodiments, R₁ may be selected from the group consisting of hydrogen, optionally substituted C₁₋₆ alkyl, and optionally substituted aryl. The alkyl may be selected from the group consisting of methyl, ethyl, propyl, isopropyl, butyl, sec-butyl, and tert-butyl, while the aryl may be phenyl or naphthyl. In other embodiments, R₁ may be a heteroaryl. In certain embodiments, R₁ may be hydrogen. In certain embodiments, R₁ is absent.

In some embodiments, X may be nitrogen. In other embodiments, Y may be NH and W may be nitrogen or CH.

In some embodiments of the compounds of Formula I or Formula XV, L is absent or is selected from the group consisting of —NHCH₂—, —NH—, and —CH₂—. In some embodiments of the compounds of Formula I or Formula XV, A is selected from the group consisting of: 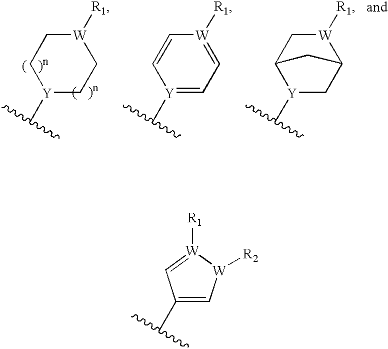
where n is selected from the group consiting of 0, 1, and 2.

Some embodiments of the compounds of Formula I, Formula II, or Formula XV, include:
2,7-Dichloro-11-(piperazin-1-yl)-5H-dibenzo[b,e][1,4]diazepine,
2-Chloro-11-(piperazin-1-yl)-5H-dibenzo[b,e][1,4]diazepine,
2,8-Dichloro-11-(piperazin-1-yl)-5H-dibenzo[b,e][1,4]diazepine,
8-Bromo-2-chloro-11-(piperazin-1-yl)-5H-dibenzo[b,e][1,4]diazepine,
2-Chloro-11-(piperazin-1-yl)-8-trifluoromethyl-5H-dibenzo[b,e][1,4]diazepine,
6-Chloro-11-(piperazin-1-yl)-8-trifluoromethyl-5H-dibenzo[b,e][1,4]diazepine,
7-Chloro-11-(piperazin-1-yl)-5H-dibenzo[b,e][1,4]diazepine,
8-Bromo-1-chloro-11-(piperazin-1-yl)-5H-dibenzo[b,e][1,4]diazepine,
8-Bromo-2-methyl-11-(piperazin-1-yl)-5H-dibenzo[b,e][1,4]diazepine,
4,8-Dichloro-11-(piperazin-1-yl)-5H-dibenzo[b,e][1,4]diazepine,
8-Chloro-2-methyl-11-(piperazin-1-yl)-5H-dibenzo[b,e][1,4]diazepine,
8-Chloro-2-fluoro-11-(piperazin-1-yl)-5H-dibenzo[b,e][1,4]diazepine,
3,8-Dichloro-11-(piperazin-1-yl)-5H-dibenzo[b,e][1,4]diazepine,
2-Bromo-8-chloro-11-(piperazin-1-yl)-5H-dibenzo[b,e][1,4]diazepine,
3,7-Dichloro-11-(piperazin-1-yl)-5H-dibenzo[b,e][1,4]diazepine,
8-Bromo-3-chloro-11-(piperazin-1-yl)-5H-dibenzo[b,e][1,4]diazepine,
3-Chloro-11-(piperazin-1-yl)-5H-dibenzo[b,e][1,4]diazepine,
3-Chloro-11-(piperazin-1-yl)-8-trifluoromethyl-5H-dibenzo[b,e][1,4]diazepine,
7-Chloro-2-methyl-11-(piperazin-1-yl)-5H-dibenzo[b,e][1,4]diazepine,
2-Methyl-11-(piperazin-1-yl)-5H-dibenzo[b,e][1,4]diazepine,
2-Methyl-11-(piperazin-1-yl)-8-trifluoromethyl-5H-dibenzo[b,e][1,4]diazepine,
8-Chloro-4-methyl-11-(piperazin-1-yl)-5H-dibenzo[b,e][1,4]diazepine,
1,8-Dichloro-11-(piperazin-1-yl)-5H-dibenzo[b,e][1,4]diazepine,
8-Bromo-5-methyl-11-(piperazin-1-yl)-5H-dibenzo[b,e][1,4]diazepine,
7,8-Dichloro-11-(piperazin-1-yl)-5H-dibenzo[b,e][1,4]diazepine,
11-(piperazin-1-yl)-8-trifluoromethyl-5H-dibenzo[b,e][1,4]diazepine,
11-(piperazin-1-yl)-5H-dibenzo[b,e][1,4]diazepine,
8-Fluoro-11-(piperazin-1-yl)-5H-dibenzo[b,e][1,4]diazepine,
11-(piperazin-1-yl)-5H-dibenzo[b,e][1,4]diazepine-8-carbonitrile,
8-Bromo-11-(piperazin-1-yl)-5H-dibenzo[b,e][1,4]diazepine,
8-Methyl-11-(piperazin-1-yl)-5H-dibenzo[b,e][1,4]diazepine,
3-Fluoro-6-piperazin-1-yl-11H-dibenzo[b,e]azepine,
2-(Trifluoromethanesulfonyloxy)-11-(piperazin-1-yl)-5H-dibenzo[b,e][1,4]diazepine,
2-(Trifluoromethanesulfonyloxy)-11-(piperazin-1-yl)-5H-dibenzo[b,e][1,4]oxazepine,
8-Chloro-2-(trifluoromethanesulfonyloxy)-11-(piperazin-1-yl)-5H-dibenzo[b,e][1,4]diazepine,
8-(Trifluoromethanesulfonyloxy)-11-(piperazin-1-yl)-5H-dibenzo[b,e][1,4]diazepine,
11-(piperazin-1-yl)-dibenzo[b,e][1,4]thiazepin,
11-(piperazin-1-yl)-2,3-dihydro-1,4-benzodioxino[6,7-b][1,4]benzothiazepin,
8-Chloro-11-[1,4]diazepam-1-yl-5H-dibenzo[b,e][1,4]diazepine,
N′-(8-Chloro-5H-dibenzo[b,e][1,4]diazepine-11-yl)-N,N-dimethyl-ethane-1,2-diamine,
N′-(8-Chloro-5H-dibenzo[b,e][1,4]diazepine-11-yl)-N,N-diethyl-ethane-1,2-diamine,
8-Chloro-11-(4-methyl-[1,4]diazepam-1-yl)-5H-dibenzo[b,e][1,4]diazepine,
8-Chloro-2-methoxy-11-(piperazin-1-yl)-5H-dibenzo[b,e][1,4]diazepine,
N′-(5H-Dibenzo[b,e][1,4]diazepine-11-yl)-N,N-dimethyl-ethane-1,2-diamine,
11-[1,4]Diazepam-1-yl-5H-dibenzo[b,e][1,4]diazepine,
N′-(8-Fluoro-5H-dibenzo[b,e][1,4]diazepine-11-yl)-N,N-dimethyl-ethane-1,2-diamine,
8-Fluoro-1-[1,4]diazepam-1-yl-5H-dibenzo[b,e][1,4]diazepine,
N′-(8-Chloro-5H-dibenzo[b,e][1,4]diazepine-11-yl)-N-methyl-ethane-1,2-diamine,
8-Chloro-11-(trans-2,5-dimethyl-piperazin-1-yl)-5H-dibenzo[b,e][1,4]diazepine,
8-Chloro-11-(3,5-dimethyl-piperazin-1-yl)-5H-dibenzo[b,e][1,4]diazepine,
8-Chloro-11-(3-methyl-piperazin-1-yl)-5H-dibenzo[b,e][1,4]diazepine,
8-Chloro-11-(3-phenyl-piperazin-1-yl)-5H-dibenzo[b,e][1,4]diazepine,
8-Chloro-5-methyl-1-(piperazin-1-yl)-5H-dibenzo[b,e][1,4]diazepine,
8-Chloro-5-benzyl-11-(piperazin-1-yl)-5H-dibenzo[b,e][1,4]diazepine,
8-Iodo-11-(piperazin-1-yl)-5H-dibenzo[b,e][1,4]diazepine,
2-Iodo-8-chloro-11-(piperazin-1-yl)-5H-dibenzo[b,e][1,4]diazepine,
8-Phenyl-11-(piperazin-1-yl)-5H-dibenzo[b,e][1,4]diazepine,
8-Chloro-11-(piperidin-1-yl)-5H-dibenzo[b,e][1,4]diazepine,
8-Chloro-11-(morpholin-4-yl)-5H-dibenzo[b,e][1,4]diazepine,
5-Allyl-8-chloro-11-(piperazin-1-yl)-5H-dibenzo[b,e][1,4]diazepine,
6-Chloro-11-(piperazin-1-yl)-5H-dibenzo[b,e][1,4]diazepine,
8-Chloro-5-piperazin-1-yl-11H-benzo[b]pyrido[2,3-e][1,4]diazepine,
2-Chloro-10-piperazin-1-yl-5H-dibenzo[b,f]azepin,
8-Chloro-11-(piperazin-1-yl)-dibenzo[b,f][1,4]thiazepine,
8-Chloro-11-(piperazin-1-yl)-dibenzo[b,f][1,4]oxazepine,
8-Chloro-11-(4-methyl-piperazin-1-yl)-dibenzo[b,f][1,4]oxazepine,
3-Chloro-6-piperazin-1-yl-11H-dibenzo[b,e]azepine,
8-Bromo-11-(piperazin-1-yl)-dibenzo[b,f][1,4]oxazepine,
11-(piperazin-1-yl)-dibenzo[b,f][1,4]oxazepine,
7-Chloro-11-(piperazin-1-yl)-dibenzo[b,f][1,4]oxazepine,
8-Chloro-3-methoxy-11-(piperazin-1-yl)-dibenzo[b,f][1,4]oxazepine,
8-Bromo-3-methoxy-11-(piperazin-1-yl)-dibenzo[b,f][1,4]oxazepine,
3-Methoxy-11-(piperazin-1-yl)-dibenzo[b,f][1,4]oxazepine,
7-Chloro-3-methoxy-11-(piperazin-1-yl)-dibenzo[b,f][1,4]oxazepine,
8-Chloro-4-methyl-11-(piperazin-1-yl)-dibenzo[b,f][1,4]oxazepine,
8-Bromo-4-methyl-11-(piperazin-1-yl)-dibenzo[b,f][1,4]oxazepine,
4-Methyl-11-(piperazin-1-yl)-dibenzo[b,f][1,4]oxazepine,
2-Bromo-8-chloro-11-(piperazin-1-yl)-dibenzo[b,f][1,4]oxazepine,
2,8-Dibromo-11-(piperazin-1-yl)-dibenzo[b,f][1,4]oxazepine,
2-Bromo-11-(piperazin-1-yl)-dibenzo[b,f][1,4]oxazepine,
2-Bromo-7-chloro-11-(piperazin-1-yl)-dibenzo[b,f][1,4]oxazepine,
11-(piperazin-1-yl)-8-trifluoromethyl-dibenzo[b,f][1,4]oxazepine,
4-Methyl-11-(piperazin-1-yl)-8-trifluoromethyl-dibenzo[b,f][1,4]oxazepine,
8-Fluoro-11-(piperazin-1-yl)-dibenzo[b,f][1,4]oxazepine,
8-Fluoro-3-methoxy-11-(piperazin-1-yl)-dibenzo[b,f][1,4]oxazepine,
8-Fluoro-4-methyl-11-(piperazin-1-yl)-dibenzo[b,f][1,4]oxazepine,
2-Bromo-8-fluoro-11-(piperazin-1-yl)-dibenzo[b,f][1,4]oxazepine,
8-Methyl-11-(piperazin-1-yl)-dibenzo[b,f][1,4]oxazepine,
3-Methoxy-8-methyl-11-(piperazin-1-yl)-dibenzo[b,f][1,4]oxazepine,
4,8-Dimethyl-11-(piperazin-1-yl)-dibenzo[b,f][1,4]oxazepine,
3-Methoxy-11-(piperazin-1-yl)-8-trifluoromethyl-dibenzo[b,f][1,4]oxazepine,
2-Bromo-11-(piperazin-1-yl)-8-trifluoromethyl-dibenzo[b,f][1,4]oxazepine,
6-Chloro-11-(piperazin-1-yl)-dibenzo[b,f][1,4]oxazepine,
2-Bromo-8-methyl-11-(piperazin-1-yl)-dibenzo[b,f][1,4]oxazepine,
7-Chloro-4-methyl-11-(piperazin-1-yl)-dibenzo[b,f][1,4]oxazepine,
8-Phenyl-11-(piperazin-1-yl)-dibenzo[b,f][1,4]oxazepine,
8-Chloro-11-(piperidin-4-yl)-5H-dibenzo[b,e][1,4]diazepine
5-Benzyl-8-chloro-11-(piperidin-4-yl)-5H-dibenzo[b,e][1,4]diazepine,
8-Bromo-5,10-dihydro-dibenzo[b,e][1,4]diazepine-11-one,
5,10-Dihydro-dibenzo[b,e][1,4]diazepine-11-one,
8-Fluoro-5,10-dihydro-dibenzo[b,e][1,4]diazepine-11-one,
8,5-Dichloro-5H-dibenzo[b,e][1,4]diazepine,
8-Chloro-11-methylsulfanyl-5H-dibenzo[b,e][1,4]diazepine
(8-Chloro-5H-dibenzo[b,e][1,4]diazepin-11-yl)-(S)-1-pyrrolidin-2-yl-methyl-amine,
1-(8-Chloro-5H-dibenzo[b,e][1,4]diazepin-11-yl)-piperidine-4-yl-amine,
1-(8-Chloro-5H-dibenzo[b,e][1,4]diazepin-11-yl)-pyrrolidin-3-yl-amine,
(8-Chloro-5H-dibenzo[b,e][1,4]diazepin-11-yl)-(R)-1-pyrrolidin-2-yl-methyl-amine,
(8-Chloro-5H-dibenzo[b,e][1,4]diazepin-11-yl)-pyrrolidin-3-yl-amine,
8-Chloro-11-(2,5-diaza-bicyclo[2.2.1]hept-2-yl)-5H-dibenzo[b,e][1,4]diazepine,
Acetidin-3-yl-8-chloro-5H-dibenzo[b,e][1,4]diazepine-11-yl)amine,
7-Bromo-4-(piperazin-1-yl)-2,3-dihydro-1H-benzo[b][1,4]diazepine,
7-Bromo-2-methyl-(piperazin-1-yl)-2,3-dihydro-1H-benzo[b][1,4]diazepine
7-Bromo-2-phenyl-4-(piperazine-1-yl)-2,3-dihydro-1H-benzo[b][1,4]diazepine,
7-Bromo-10-(piperazin-1-yl)-1,2,3,3a,4,10a-hexahydro-benzo[b]cyclopenta[e][1,4]diazepine,
8-Chloro-11-(4-fluorobenzyl)-5H-dibenzo[b,e][1,4]diazepine,
8-Chloro-11-(4-fluorophenyl)-5H-dibenzo[b,e][1,4]diazepine,
8-Chloro-11-(4-nonylphenyl)-5H-dibenzo[b,e][1,4]diazepine,
8-Chloro-11-(pyridin-4-yl)-5H-dibenzo[b,e][1,4]diazepine, and
8-Chloro-11-(1H-pyrazol-4-yl)-5H-dibenzo[b,e][1,4]diazepine.

In some embodiments, the compound of Formula I is N-desmethylclozapine (NDMC), 8-chloro-11-(1-piperazinyl)-5H-dibenzo[b,e][1,4]diazepine, which has the following structure:

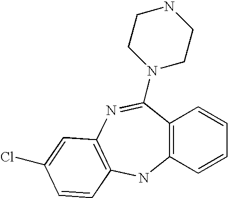

In some embodiments, the compound of Formula I does not include N-desmethylclozapine.

The term “aromatic” refers to an aromatic group which has at least one ring having a conjugated pi electron system and includes both carbocyclic aryl (e.g., phenyl) and heterocyclic aryl groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon. The term “heteroaromatic” refers to an aromatic group which contains at least one heterocyclic ring.

As used herein, the term “alkyl” refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds of the invention may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl.

The alkyl group may be substituted or unsubstituted. When substituted, the substituent group(s) is(are) one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. Wherever a substituent is described as being “optionally substituted” that substitutent may be substituted with one of the above substituents.

The substituent “R” appearing by itself and without a number designation refers to a substituent selected from the group consisting of of alkyl, cycloalkyl, aryl, heteroaryl (bonded through a ring carbon) and heteroalicyclic (bonded through a ring carbon).

An “O-carboxy” group refers to a RC(═O)O— group, where R is as defined herein.

A “C-carboxy” group refers to a —C(═O)OR groups where R is as defined herein.

An “acetyl” group refers to a —C(═O)CH₃, group.

A “trihalomethanesulfonyl” group refers to a X₃CS(═O)₂— group where X is a halogen.

A “cyano” group refers to a —CN group.

An “isocyanato” group refers to a —NCO group.

A “thiocyanato” group refers to a —CNS group.

An “isothiocyanato” group refers to a —NCS group.

A “sulfinyl” group refers to a —S(═O)—R group, with R as defined herein.

A “S-sulfonamido” group refers to a —S(═O)₂NR, group, with R as defined herein.

A “N-sulfonamido” group refers to a RS(═O)₂NH— group with R as defined herein.

A “trihalomethanesulfonamido” group refers to a X₃CS(═O)₂NR— group with X and R as defined herein.

An “O-carbamyl” group refers to a —OC(═O)—NR, group-with R as defined herein.

An “N-carbamyl” group refers to a ROC(═O)NH— group, with R as defined herein.

An “O-thiocarbamyl” group refers to a —OC(═S)—NR, group with R as defined herein.

An “N-thiocarbamyl” group refers to an ROC(═S)NH— group, with R as defined herein.

A “C-amido” group refers to a —C(═O)—NR₂ group with R as defined herein.

An “N-amido” group refers to a RC(═O)NH— group, with R as defined herein.

The term “perhaloalkyl” refers to an alkyl group where all of the hydrogen atoms are replaced by halogen atoms.

The term “acylalkyl” refers to a RC(═O)R′— group, with R as defined herein, and R′ being a diradical alkylene group. Examples of acylalkyl, without limitation, may include CH₃C(═O)CH₂—, CH₃C(═O)CH₂CH₂—, CH₃CH₂C(═O)CH₂CH₂—, CH₃C(═O)CH₂CH₂CH₂—, and the like.

Unless otherwise indicated, when a substituent is deemed to be “optionally subsituted,” it is meant that the subsitutent is a group that may be substituted with one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. The protecting groups that may form the protective derivatives of the above substituents are known to those of skill in the art and may be found in references such as Greene and Wuts, above.

In the present context, the term “cycloalkyl” is intended to cover three-, four-, five-, six-, seven-, and eight- or more membered rings comprising carbon atoms only. A cycloalkyl can optionally contain one or more unsaturated bonds situated in such a way, however, that an aromatic pi-electron system does not arise. Some examples of “cycloalkyl” are the carbocycles cyclopropane, cyclobutane, cyclopentane, cyclopentene, cyclopentadiene, cyclohexane, cyclohexene, 1,3-cyclohexadiene, 1,4-cyclohexadiene, cycloheptane, or cycloheptene.

The term “heterocyclyl” is intended to mean three-, four-, five-, six-, seven-, and eight- or more membered rings wherein carbon atoms together with from 1 to 3 heteroatoms constitute said ring. A heterocyclyl can optionally contain one or more unsaturated bonds situated in such a way, however, that an aromatic pi-electron system does not arise. The heteroatoms are independently selected from oxygen, sulfur, and nitrogen.

A heterocyclyl can further contain one or more carbonyl or thiocarbonyl functionalities, so as to make the definition include oxo-systems and thio-systems such as lactams, lactones, cyclic imides, cyclic thioimides, cyclic carbamates, and the like.

Heterocyclyl rings can optionally also be fused to aryl rings, such that the definition includes bicyclic structures. Typically such fused heterocyclyl groups share one bond with an optionally substituted benzene ring. Examples of benzo-fused heterocyclyl groups include, but are not limited to, benzimidazolidinone, tetrahydroquinoline, and methylenedioxybenzene ring structures.

Some examples of “heterocyclyls” include, but are not limited to, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Binding to the heterocycle can be at the position of a heteroatom or via a carbon atom of the heterocycle, or, for benzo-fused derivatives, via a carbon of the benzenoid ring.

In the present context the term “aryl” is intended to mean a carbocyclic aromatic ring or ring system. Moreover, the term “aryl” includes fused ring systems wherein at least two aryl rings, or at least one aryl and at least one C₃₋₈-cycloalkyl share at least one chemical bond. Some examples of “aryl” rings include optionally substituted phenyl, naphthalenyl, phenanthrenyl, anthracenyl, tetralinyl, fluorenyl, indenyl, and indanyl. The term “aryl” relates to aromatic, including, for example, benzenoid groups, connected via one of the ring-forming carbon atoms, and optionally carrying one or more substituents selected from heterocyclyl, heteroaryl, halo, hydroxy, amino, cyano, nitro, alkylamido, acyl, C₁₋₆ alkoxy, C₁₋₆ alkyl, C₁₋₆ hydroxyalkyl, C₁₋₆ aminoalkyl, C₁₋₆ alkylamino, alkylsulfenyl, alkylsulfinyl, alkylsulfonyl, sulfamoyl, or trifluoromethyl. The aryl group can be substituted at the para and/or meta positions. In other embodiments, the aryl group can be substituted at the ortho position. Representative examples of aryl groups include, but are not limited to, phenyl, 3-halophenyl, 4-halophenyl, 3-hydroxyphenyl, 4-hydroxyphenyl, 3-aminophenyl, 4-aminophenyl, 3-methylphenyl, 4-methylphenyl, 3-methoxyphenyl, 4-methoxyphenyl, 4-trifluoromethoxyphenyl 3-cyanophenyl, 4-cyanophenyl, dimethylphenyl, naphthyl, hydroxynaphthyl, hydroxymethylphenyl, trifluoromethylphenyl, alkoxyphenyl, 4-morpholin-4-ylphenyl, 4-pyrrolidin-1-ylphenyl, 4-pyrazolylphenyl, 4-triazolylphenyl, and 4-(2-oxopyrrolidin-1-yl)phenyl.

In the present context, the term “heteroaryl” is intended to mean a heterocyclic aromatic group where one or more carbon atoms in an aromatic ring have been replaced with one or more heteroatoms selected from the group comprising nitrogen, sulfur, phosphorous, and oxygen.

Furthermore, in the present context, the term “heteroaryl” comprises fused ring systems wherein at least one aryl ring and at least one heteroaryl ring, at least two heteroaryl rings, at least one heteroaryl ring and at least one heterocyclyl ring, or at least one heteroaryl ring and at least one cycloalkyl ring share at least one chemical bond.

The term “heteroaryl” is understood to relate to aromatic, C₃₋₈ cyclic groups further containing one oxygen or sulfur atom or up to four nitrogen atoms, or a combination of one oxygen or sulfur atom with up to two nitrogen atoms, and their substituted as well as benzo- and pyrido-fused derivatives, for example, connected via one of the ring-forming carbon atoms. Heteroaryl groups can carry one or more substituents, selected from halo, hydroxy, amino, cyano, nitro, alkylamido, acyl, C₁₋₆-alkoxy, C₁₋₆-alkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl. In some embodiments, heteroaryl groups can be five- and six-membered aromatic heterocyclic systems carrying 0, 1, or 2 substituents, which can be the same as or different from one another, selected from the list above. Representative examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quionoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline. In some embodiments, the substituents are halo, hydroxy, cyano, O—C₁₋₆-alkyl, C₁₋₆-alkyl, hydroxy-C₁₋₆-alkyl, and amino-C₁₋₆-alkyl.

The compounds of Formula I, II, or XV may be used for the purpose of controlling the positive (e.g., hallucinations and delusion) and negative (e.g., apathy, social withdrawal, anhedonia) symptoms of schizophrenia or related psychosis. In one embodiment, the psychosis is induced by exposure of the subject or one or more medications. In one embodiment, the compounds are administered to ameliorate one or more symptoms associated with psychosis is essentially free of clozapine. By “essentially free of clozapine,” it is meant that no appreciable amount of clozapine may be detected in the blood stream of the subject at the same time that the administered compound is detectable in the blood stream of the subject. In one embodiment, the amount of any clozapine administered with comopund is low enough such that the combined compound of Formula I, II, or XV and clozapine administered result in a net agonism at dopamine receptors. In one embodiment, the net agonism is a partial agonism. In one embodiment, some amount of clozapine is administered but it is low enough such that the combined compound and clozapine administered result in a net agonism at dopamine receptors. In one embodiment, the ratio of the compound to clozapine is high enough to have a beneficial effect due to net agonism at dopamine receptors. In various embodiments, the ratio of the compound to clozapine is at least about 100:1, 50:1, 10:1, 9:1, 7:1, 5:1, or 3:1.

In another embodiment, the present invention relates to the use of compounds of Formula I, II, or XV in human subjects to ameliorate one or more symptoms associated with affective disorders, including major depression, mania, bipolar disorder, and suicide. In this respect, the compounds may be used for the purpose of controlling the symptoms observed during major depression or manic depression. In one embodiment, the compound administered to ameliorate one or more symptoms associated with affective disorders is essentially free of clozapine. In one embodiment, the amount of any clozapine administered with the compound is low enough such that the combined compound and clozapine administered result in a net agonism at dopamine receptors. In one embodiment, the net agonism is a partial agonism. In one embodiment, some amount of clozapine is administered but it is low enough such that the combined compound and clozapine administered result in a net agonism at dopamine receptors.

In another embodiment, the present invention relates to the use of a compound of Formula I, II, or XV in human subjects to ameliorate one or more symptoms associated with dementia, such as is caused by Alzheimer's Disease and related neurodegenerative disorders. In this respect, the compound may be used for the purpose of improving the cognitive deficits and controlling the associated behavioral abnormalities observed in degenerative dementias. In one embodiment, the compound administered to ameliorate one or more symptoms associated with dementia is essentially free of clozapine. In one embodiment, the amount of any clozapine administered with the compound is low enough such that the combined compound and clozapine administered result in a net agonism at dopamine receptors. In one embodiment, the net agonism is a partial agonism. In one embodiment, some amount of clozapine is administered but it is low enough such that the combined compound and clozapine administered result in a net agonism at dopamine receptors.

In another embodiment, the present invention relates to the use of a compound of Formula I, II, or XV in human subjects to ameliorate one or more symptoms associated with neuropathic pain. In this respect, the compound may be used for the purpose of controlling the dysthesthetic, hyperalgesic, and other altered nociceptive symptoms observed in neuropathic pain states regardless of their etiology. In one embodiment, the compound administered to ameliorate one or more symptoms associated with neuropathic pain is essentially free of clozapine. In one embodiment, the amount of any clozapine administered with the compound is low enough such that the combined compound and clozapine administered result in a net agonism at dopamine receptors. In one embodiment, the net agonism is a partial agonism. In one embodiment, some amount of clozapine is administered but it is low enough such that the combined compound and clozapine administered result in a net agonism at dopamine receptors.

In another embodiment, the present invention relates to the use of a compound of Formula I, II, or XV in human subjects to ameliorate one or more symptoms associated with glaucoma. In this respect, the compound may be used for the purpose of controlling the raised intra-ocular pressure observed in glaucoma, regardless of its etiology. In one embodiment, the compound administered to ameliorate one or more symptoms associated with glaucoma is essentially free of clozapine. In one embodiment, the amount of any clozapine administered with the compound is low enough such that the combined compound and clozapine administered result in a net agonism at dopamine receptors. In one embodiment, the net agonism is a partial agonism. In one embodiment, some amount of clozapine is administered but it is low enough such that the combined compound and clozapine administered result in a net agonism at dopamine receptors.

In one embodiment, a compound of Formula I, II, or XV is administered to a human subject in order to ameliorate one or more symptoms associated with EPS and/or TD. In one embodiment, the compound administered to ameliorate one or more symptoms associated with EPS and/or TD is essentially free of clozapine. In one embodiment, the EPS and/or TD are caused by exposure of the subject to one or more medications, such as an anti-psychotic medication.

In one embodiment, a compound of Formula I, II, or XV is administered to a human subject that is refractory to other treatments due to a propensity of the subject to develop EPS and/or TD upon administration of the treatment. Thus, in some embodiments, a subject is identified as having a propensity to developing EPS and/or TD and then administered a compound of Formula I, II, or XV. In one embodiment, the compound is administered essentially free of clozapine. In one embodiment, the amount of any clozapine administered with the compound is low enough such that the combined compound and clozapine administered result in a net agonism at dopamine receptors. In one embodiment, the net agonism is a partial agonism. In one embodiment, some amount of clozapine is administered but it is low enough such that the combined compound and clozapine administered result in a net agonism at dopamine receptors.

In one embodiment, a compound of Formula I, II, or XV is administered to effect dopamine stabilization in a subject. In one embodiment, the compound is administered to effect stabilization of the D2 receptor.

In one embodiment, D2 receptors are modulated by contacting the D2 receptors with a compound of Formula I, II, or XV. In one embodiment, the D2 receptors are contacted with the composition essentially free of clozapine. In one embodiment, the amount of any clozapine administered with the compound is low enough such that the combined compound and clozapine administered result in a net agonism at the D2 receptors. In one embodiment, the net agonism is a partial agonism. In one embodiment, some amount of clozapine is administered but it is low enough such that the combined compound and clozapine administered result in a net agonism at dopamine receptors.

In one embodiment, D3 receptors are modulated by contacting the D3 receptors with a compound of Formula I, II, or XV. In one embodiment, the D3 receptors are contacted with a composition essentially free of clozapine. In one embodiment, the amount of any clozapine administered with the compound is low enough such that the combined compound and clozapine administered result in a net agonism at the D3 receptors. In one embodiment, the net agonism is a partial agonism. In one embodiment, some amount of clozapine is administered but it is low enough such that the combined compound and clozapine administered result in a net agonism at dopamine receptors.

In one embodiment, one or more symptoms of a condition associated with a dopamine receptor are ameliorated by administering a compound of Formula I, II, or XV to a subject. In one embodiment, the compound is administered essentially free of clozapine. In one embodiment, the amount of any clozapine administered with the compound is low enough such that the combined compound and clozapine administered result in a net agonism at dopamine receptors. In one embodiment, the net agonism is a partial agonism. In one embodiment, some amount of clozapine is administered but it is low enough such that the combined NDMC and clozapine administered result in a net agonism at dopamine receptors.

In some embodiments, a compound of Formula I, II, or XV may be used as an adjunctive therapy with known drugs to reduce the dosage required of these traditional drugs, and thereby reduce their side effects. Thus, in one embodiment, the compound is administered to a subject in combination with one or more agents. In some embodiments, the one or more additional agents are administered at a dosage that is less than the dosage that would be typically used if the other agents were administered alone. In one embodiment, the one or more agents are administered at a dosage level that is 75% or less of the typically used dosage. In one embodiment, the one or more agents are administered at a dosage level that is 50% or less of the typically used dosage. In one embodiment, the one or more agents are administered at a dosage level that is 25% or less of the typically used dosage.

In some embodiments, a compound of Formula I, II, or XV is administered in combination with one or more additional therapeutic agents. The additional therapeutic agents can include, but are not limited to, a neuropsychiatric agent. As used herein, a “neuropsychiatric agent” refers to a compound, or a combination of compounds, that affects the neurons in the brain either directly or indirectly, or affects the signal transmitted to the neurons in the brain. Neuropsychiatric agents, therefore, may affect a person's psyche, such as the person's mood, perception, nociception, cognition, alertness, memory, etc. In certain embodiments, the neuropsychiatric agent may be selected from the group consisting of monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin 1A agonists, antiepileptic and peripherally acting muscarinic antagonists.

In some embodiments, the antipsychotic agent may be selected from the group consisting of a phenothiazine, phenylbutylpiperadine, debenzapine, benzisoxidil, and a salt of lithium. The phenothiazine group of compounds may be selected from the group consisting of chlorpromazine (Thorazine®), mesoridazine (Serentil®), prochlorperazine (Compazine®), and thioridazine (Mellaril®). The phenylbutylpiperadine group of compounds may be selected from the group consisting of haloperidol (Haldol®), and pimozide (Orap®). The debenzapine group of compounds may be selected from the group consisting of clozapine (Clozaril®), loxapine (Loxitane®), olanzapine (Zyprexa®) and quetiapine (Seroquel®). The benzisoxidil group of compounds may be selected from the group consisting of resperidone (Resperidal®) and ziprasidone (Geodon®). The salt of lithium may be lithium carbonate. In some embodiments, the antipsychotic agent may be selected from the group consisting of Aripiprazole (Abilify), Clozapine, Clozaril, Compazine, Etrafon, Geodon, Haldol, Inapsine, Loxitane, Mellaril, Moban, Navane, Olanzapine (Zyprexa), Orap, Permitil, Prolixin, Phenergan, Quetiapine (Seroquel), Reglan, Risperdal, Serentil, Seroquel, Stelazine, Taractan, Thorazine, Triavil, Trilafon, and Zyprexa, or pharmaceutically acceptable salts thereof.

In certain embodiments, the selective serotonin reuptake inhibitor is selected from the group consisting of fluoxetine, fluvoxamine, sertraline, paroxetine, citalopram, escitalopram, sibutramine, duloxetine, and venlafaxine, and pharmaceutically acceptable salts or prodrugs thereof.

In other embodiments, the norepinephrine reuptake inhibitor is selected from the group consisting of thionisoxetine and reboxetine.

In further embodiments, the dopamine agonist is selected from the group consisting of cabergoline, amantadine, lisuride, pergolide, ropinirole, pramipexole, and bromocriptine.

In another embodiment, the inverse serotonin 2A agonist is N-(1-methylpiperidin-4-yl)-N-(4-flourophenylmethyl)-N′-(4-(2-methylpropyloxy)phenylmethyl)carbamide, MDL 100,907, SR-43694B (eplivanserin), ritanserin, ketanserin, mianserin, cinanserin, mirtazepine, cyproheptadine and cinnarizine.

In another aspect, the present disclosure is directed to a method of treating neuropsychiatric disorder in a patient comprising identifying a patient in need thereof and administering to said patient a therapeutically effective amount of a pharmaceutical composition comprising a compound of Formula I, II, or XV and a neuropsychiatric agent. In yet another aspect, the present disclosure is directed to a method of treating a neuropsychiatric disorder in a patient comprising identifying a patient in need thereof and administering to said patient a therapeutically effective amount of a compound of Formula I, II, or XV and a therapeutically effective amount of a neuropsychiatric agent.

In some embodiments, a compound of Formula I, II, or XV and additional therapeutic agent(s) are administered nearly simultaneously. These embodiments include those in which the compounds are in the same administrable composition, i.e., a single tablet, pill, or capsule, or a single solution for intravenous injection, or a single drinkable solution, or a single dragee formulation or patch, contains the compounds. The embodiments also include those in which each compound is in a separate administrable composition, but the patient is directed to take the separate compositions nearly simultaneously, i.e., one pill is taken right after the other or that one injection of one compound is made right after the injection of another compound, etc.

In other embodiments, one of a compound of Formula I, II, or XV and an additional therapeutic compound is administered first and then the other one of a compound of Formula I, II, or XV and the additional therapeutic compound is administered second. In these embodiments, the patient may be administered a composition comprising one of the compounds and then at some time, a few minutes later, a few hours later, or at some other later desired time be administered another composition comprising the other one of the compounds. Also included in these embodiments are those in which the patient is administered a composition comprising one of the compounds on a routine or continuous basis while receiving a composition comprising the other compound occasionally.

By administration in “combination,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered i.v. In one advantageous embodiment, the pharmacokinetics of the two or more agents are substantially the same.

In some embodiments of combination administration, a compound of Formula I, II, or XV is administered in combination with another therapeutic agent, wherein at least a portion of the compound is administered by directly introducing the compound to a subject. Thus, for example, clozapine may be administered in combination with NDMC wherein both clozapine and NDMC are directly administered to a subject. A portion of the NDMC administered to the patient will be due to metabolism of clozapine. However, another portion of NDMC will be due to direct administration of NDMC. In one embodiment, directly introducing NDMC to a subject may be accomplished by the subject orally ingesting NDMC. In one embodiment, directly introducing NDMC to a subject may be accomplished by intravenously injecting NDMC into the subject.

In some embodiments, prodrugs, metabolites, stereoisomers, and pharmaceutically acceptable salts of a compound of Formula I, II, or XV disclosed herein are provided.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety.

The term “pro-drug ester” refers to derivatives of the compounds disclosed herein formed by the addition of any of several ester-forming groups that are hydrolyzed under physiological conditions. Examples of pro-drug ester groups include pivoyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference in their entirety.

Metabolites of the compounds disclosed herein include active species that are produced upon introduction of the compounds into the biological milieu.

Where the compounds disclosed herein have at least one chiral center, they may exist as a racemate or as enantiomers. It should be noted that all such isomers and mixtures thereof are included in the scope of the present invention. Furthermore, some of the crystalline forms for the compounds of disclosed herein may exist as polymorphs. Such polymorphs are included in one embodiment of the present invention. In addition, some of the compounds of the present invention may form solvates with water (i.e., hydrates) or common organic solvents. Such solvates are included in one embodiment of the present invention.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine, lysine, and the like.

If the manufacture of pharmaceutical formulations involves intimate mixing of the pharmaceutical excipients and the active ingredient in its salt form, then it may be desirable to use pharmaceutical excipients which are non-basic, that is, either acidic or neutral excipients.

In various embodiments, the compounds disclosed herein can be used alone, in combination with other compounds disclosed herein, or in combination with one or more other agents active in the therapeutic areas described herein.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

Any amine, hydroxy, or carboxyl side chain on the compounds of the present invention can be esterified or amidified. The procedures and specific groups to be used to achieve this end are known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

The terms “purified,” “substantially purified,” and “isolated” as used herein refer to compounds disclosed herein being free of other, dissimilar compounds with which the compounds of the invention are normally associated in their natural state, so that the compounds of the invention comprise at least 0.5%, 1%, 5%, 10%, or 20%, and most preferably at least 50% or 75% of the mass, by weight, of a given sample.

An “agonist” is defined as a compound that increases the basal activity of a receptor (i.e. signal transduction mediated by the receptor).

An “antagonist” is defined as a compound which blocks the action of an agonist on a receptor.

An “inverse agonist” is defined as a compound which reduces, or suppresses the basal activity of a receptor.

A partial agonist is defined as an agonist that displays limited, or less than complete, activity compared to an agonist.

The term “subject” refers to an animal, preferably a mammal, and most preferably a human, who is the object of treatment, observation or experiment.

The term “therapeutically effective amount” is used to indicate an amount of an active compound, or pharmaceutical agent, that elicits the biological or medicinal response indicated. This response may occur in a tissue, system, animal or human that is being sought by a researcher, veterinarian, medical doctor or other clinician, and includes alleviation of the symptoms of the disease being treated.

Methods of Preparation

In some embodiments, compounds of Formula V or Formula VI: 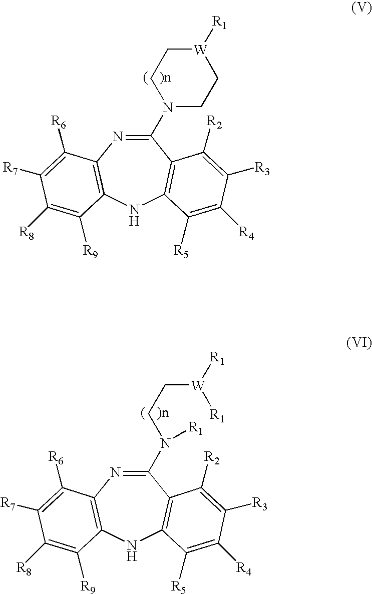
are synthesized by reacting a compound of Formula VII 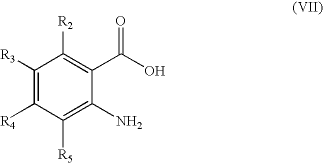
with a compound of Formula VIII 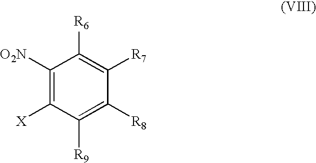
to form a fused ring compound of Formula IX, 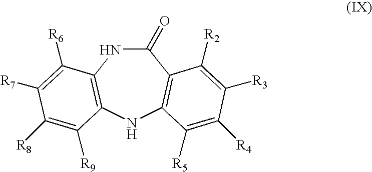
and reacting the compound of Formula IX with a compound of Formula X 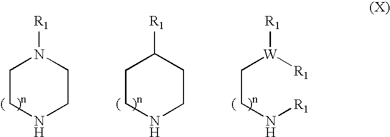
to obtain a compound of Formula V or VI; wherein X is a halogen; and R₁-R₉ are as defined herein. In some embodiments, the compound of Formula V synthesized according to the disclosed method is clozapine while in other embodiments, the compound is N-desmethylclozapine. In certain other embodiments, the compound of Formula V synthesized according to the disclosed method does not include clozapine or N-desmethylclozapine.

Consistent with this aspect, Schemes 1 and 2 depict the synthesis of some of the compounds disclosed herein. The first series of steps generating the intermediate lactam have been described by, inter alia, Liao et al. J. Med. Chem. 1997, 40, 4146-4153. The last step has been described by e.g. Liao et al. J. Med. Chem. 1999, 42, 2235-2244. Both of these references are hereby incorporated herein by reference in their entirety, including any drawings.

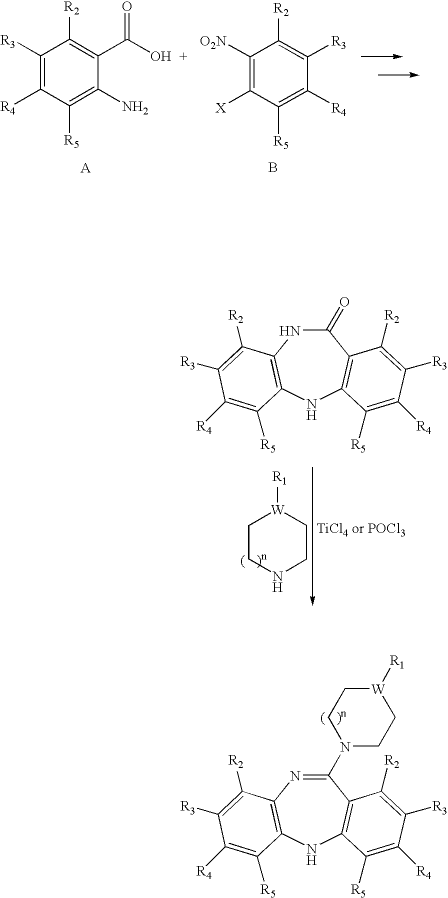

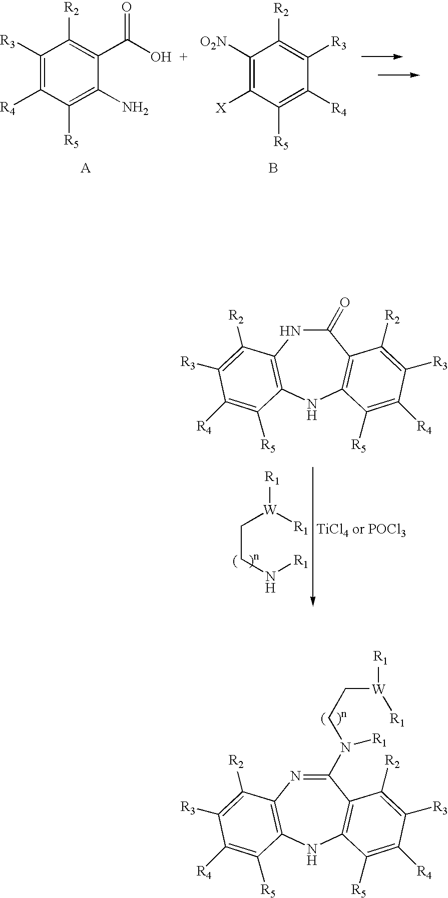

In certain embodiments of the invention the building blocks A and B are selected from but not limited to

A

| | |
|---|---|
| 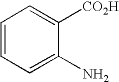 | 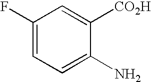 |
| 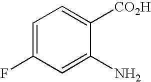 | 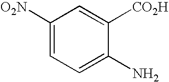 |
| 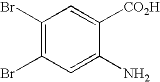 | 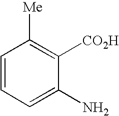 |
| 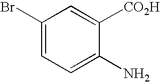 | 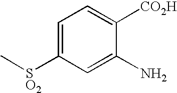 |
| 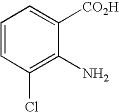 | 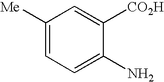 |
| 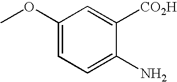 | 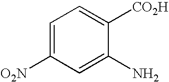 |
| 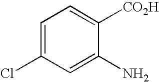 | 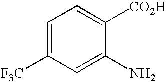 |
| 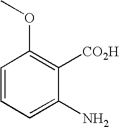 | 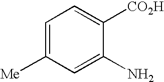 |
| 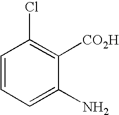 | 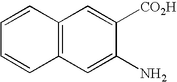 |
| 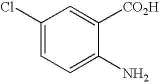 | 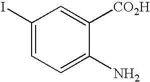 |

B

| | |
|---|---|
| 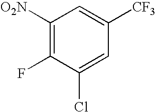 | 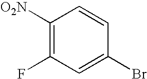 |
| 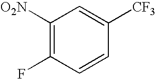 | 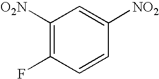 |
| 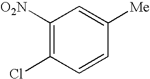 | 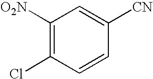 |
| 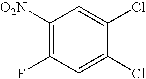 | 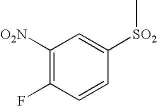 |
| 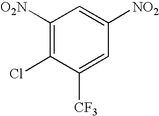 | 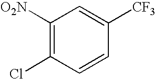 |

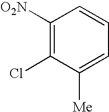

Dibenzo[b,e][1,4]diazepine compounds may be formed by reacting a compound of Formula VII,

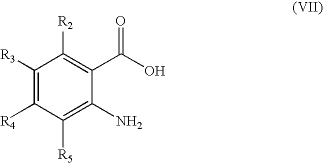

with a compound of Formula VIII and

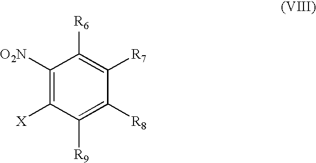

a compound of Formula XI,

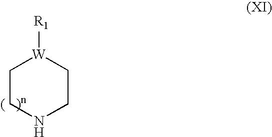

wherein X is a halogen; W is nitrogen, CH, oxygen, or sulfur; n is 1, 2, 3, or 4 and R₁-R₉ are as defined herein. In some embodiments, the combinatorial library includes clozapine and/or N-desmethylclozapine. In certain other embodiments, the combinatorial library does not include clozapine or N-desmethylclozapine.

In another embodiment, dibenzo[b,e][1,4]diazepine compounds may be formed by reacting a compound of Formula VII,

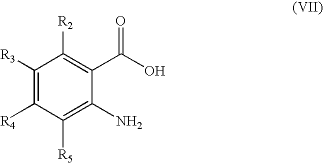

with a compound of Formula VIII and

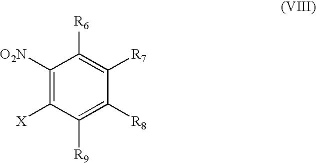

a compound of Formula XII,

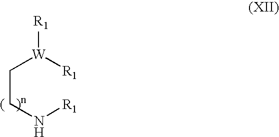

wherein X is a halogen; W is nitrogen, CH, oxygen, or sulfur; n is 1, 2, 3, or 4; and R₁-R₉ are as defined herein.

NDMC may be synthesized by methods described below, or by modification of these methods. Ways of modifying the methodology include, among others, temperature, solvent, reagents etc., and will be obvious to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and Greene & Wuts, Protective Groups in Organic Synthesis, John Wiley & Sons, 1991, which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety.

N-desmethylclozapine (I) (NDMC) may be prepared as previously described (28) and as presented in Scheme I. The dibenzo-diazepine-lactam precursor (II) may be converted to the thiolactam (III) using phosphorus pentasulfide, followed by alkylation with e.g. dimethyl sulfate to give the imino thioether (IV). Aminolysis of the thioether with an excess of piperazine gives the desired N-desmethylclozapine (I). Alternatively, the dibenzo-diazepine-lactam (II) may be converted into the imino-chloride (V) by treatment with a halogenating agent such as phosphorus pentachloride. The product (V) may be converted to N-desmethylclozapine (I) by reaction with piperazine.

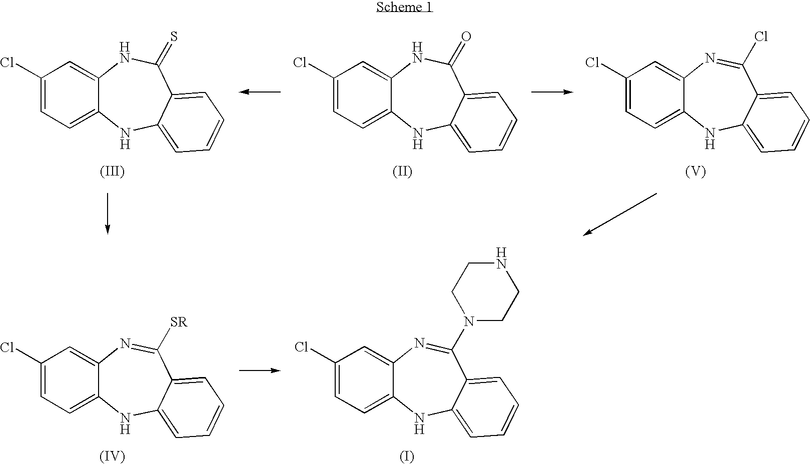

Where the processes for the preparation of the compounds disclosed herein give rise to mixtures of stereoisomers, such isomers may be separated by conventional techniques such as preparative chiral chromatography. The compounds may be prepared in racemic form or individual enantiomers may be prepared by stereoselective synthesis or by resolution. The compounds may be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid, followed by fractional crystallization and regeneration of the free base. The compounds may also be resolved using a chiral auxiliary by formation of diastereomeric derivatives such as esters, amides or ketals followed by chromatographic separation and removal of the chiral auxiliary.

Pharmaceutical Compositions

In another aspect, the present disclosure relates to a pharmaceutical composition comprising a physiologically acceptable surface active agents, carriers, diluents, excipients, smoothing agents, suspension agents, film forming substances, and coating assistants, or a combination thereof; and a compound disclosed herein. Acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives, stabilizers, dyes, sweeteners, fragrances, flavoring agents, and the like may be provided in the pharmaceutical composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium methasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, topical, or intestinal administration; parenteral delivery, including intramuscular, subcutaneous, intravenous, intramedullary injections, as well as intrathecal, direct intraventricular, intraperitoneal, intranasal, or intraocular injections. The compounds can also be administered in sustained or controlled release dosage forms, including depot injections, osmotic pumps, pills, transdermal (including electrotransport) patches, and the like, for prolonged and/or timed, pulsed administration at a predetermined rate.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations (for example, liposomes), may be utilized.

For transmucosal administration, penetrants appropriate to the barrier to be permeated may be used in the formulation.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Further disclosed herein are various pharmaceutical compositions well known in the pharmaceutical art for uses that include intraocular, intranasal, and intraauricular delivery. Suitable penetrants for these uses are generally known in the art. Pharmaceutical compositions for intraocular delivery include aqueous ophthalmic solutions of the active compounds in water-soluble form, such as eyedrops, or in gellan gum (Shedden et al., Clin. Ther., 23(3):440-50 (2001)) or hydrogels (Mayer et al., Ophthalmologica, 210(2):101-3 (1996)); ophthalmic ointments; ophthalmic suspensions, such as microparticulates, drug-containing small polymeric particles that are suspended in a liquid carrier medium (Joshi, A., J. Ocul. Pharmacol., 10(1):29-45 (1994)), lipid-soluble formulations (Alm et al., Prog. Clin. Biol. Res., 312:447-58 (1989)), and microspheres (Mordenti, Toxicol. Sci., 52(1):101-6 (1999)); and ocular inserts. All of the above-mentioned references, are incorporated herein by reference in their entireties. Such suitable pharmaceutical formulations are most often and preferably formulated to be sterile, isotonic and buffered for stability and comfort. Pharmaceutical compositions for intranasal delviery may also include drops and sprays often prepared to simulate in many respects nasal secretions to ensure maintenance of normal ciliary action. As disclosed in Remington's Pharmaceutical Sciences, 18th Ed., Mack Publishing Co., Easton, Pa. (1990), which is incorporated herein by reference in its entirety, and well-known to those skilled in the art, suitable formulations are most often and preferably isotonic, slightly buffered to maintain a pH of 5.5 to 6.5, and most often and preferably include antimicrobial preservatives and appropriate drug stabilizers. Pharmaceutical formulations for intraauricular delivery include suspensions and ointments for topical application in the ear. Common solvents for such aural formulations include glycerin and water.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For hydrophobic compounds, a suitable pharmaceutical carrier may be a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Agents intended to be administered intracellularly may be administered using techniques well known to those of ordinary skill in the art. For example, such agents may be encapsulated into liposomes. All molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. The liposome may be coated with a tissue-specific antibody. The liposomes will be targeted to and taken up selectively by the desired organ. Alternatively, small hydrophobic organic molecules may be directly administered intracellularly.

Additional therapeutic or diagnostic agents may be incorporated into the pharmaceutical compositions. Alternatively or additionally, pharmaceutical compositions may be combined with other compositions that contain other therapeutic or diagnostic agents.

Methods of Administration

The compounds or pharmaceutical compositions may be administered to the patient by any suitable means. Non-limiting examples of methods of administration include, among others, (a) administration though oral pathways, which administration includes administration in capsule, tablet, granule, spray, syrup, or other such forms; (b) administration through non-oral pathways such as rectal, vaginal, intraurethral, intraocular, intranasal, or intraauricular, which administration includes administration as an aqueous suspension, an oily preparation or the like or as a drip, spray, suppository, salve, ointment or the like; (c) administration via injection, subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; as well as (e) administration topically; as deemed appropriate by those of skill in the art for bringing the compound of the invention into contact with living tissue.

Pharmaceutical compositions suitable for administration include compositions where the active ingredients are contained in an amount effective to achieve its intended purpose. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage may range broadly, depending upon the desired affects and the therapeutic indication. Typically, dosages may be between about 10 microgram/kg and 100 mg/kg body weight, preferably between about 100 microgram/kg and 10 mg/kg body weight. Alternatively dosages may be based and calculated upon the surface area of the patient, as understood by those of skill in the art.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, which is hereby incorporated herein by reference in its entirety, with particular reference to Ch. 1, p. 1). Typically, the dose range of the composition administered to the patient can be from about 0.0001 to 25 mg/kg of the patient's body weight. Preferably, the range is about 0.001 to 10 mg/kg of body weight, and especially from about 0.001 mg/kg to 1 mg/kg body weight. The dosage may be a single one or a series of two or more given in the course of one or more days, as is needed by the patient. In instances where human dosages for compounds have been established for at least some condition, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. The daily dosage regimen for an adult human patient may be, for example, an oral dose of between 0.01 mg and 2000 mg of each active ingredient, preferably between 1 mg and 500 mg, e.g. 5 to 200 mg. In other embodiments, an intravenous, subcutaneous, or intramuscular dose of each active ingredient of between 0.01 mg and 100 mg, preferably between 0.1 mg and 60 mg, e.g. 1 to 40 mg is used. In cases of administration of a pharmaceutically acceptable salt, dosages may be calculated as the free base. In some embodiments, the composition is administered 1 to 4 times per day. Alternatively the compositions of the invention may be administered by continuous intravenous infusion, preferably at a dose of each active ingredient up to 1000 mg per day. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections. In some embodiments, the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

### EXAMPLES

### Example 1

### General Procedure 1 (GP1)

A mixture of an aminobenzoic acid (1 eq.), a 2-fluoronitrobenezene (3 eq.) and Cs₂CO₃ (3 eq.) in DMF was heated to 140° C. for 1 hour, and then allowed to obtain room temperature. The mixture was diluted with water and washed with EtOAc (2×).

EtOH and Na₂S₂O₄ (5 eq.) was added to the aqueous phase and the resulting mixture was stirred for 1 h. Aqueous HCl (2 M) was added to the mixture and then the aqueous phase was extracted with EtOAc (3×) and the combined organic phases were concentrated.

The residue was taken up in CH₂Cl₂ and 1-ethyl-3-(3-dimethylaminopropyl)carbodiimide hydrochloride (3 eq.) was added and the resulting mixture was stirred at room temperature for 1 h, and then concentrated. The residue was diluted with EtOAc, washed with aqueous NaOH (2 M) and concentrated.

The residue was taken up in dioxane and added to a mixture of TiCl₄ (1.1 eq., 1 M in toluene) and piperazine (5 eq.) in dioxane at 50° C. The resulting mixture was stirred at 100° C. over night, and then allowed to obtain room temperature. Aqueous HCl (2 M) was added to the mixture until the solution became acidic and then the aqueous phase was extracted with EtOAc (2×). Aqueous NaOH (2 M) was added to the aqueous phase until a basic solution was obtained and the resulting suspension was extracted with EtOAc (3×). The combined organic phases were concentrated and purified by HPLC.

### Example 2

### 2,7-Dichloro-11-(piperazin-1-yl)-5H-dibenzo[b,e][1,4]diazepine (166JO85F1)

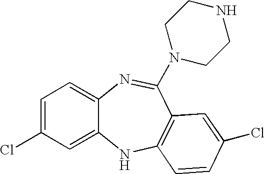

4-Chloro-2-fluoronitrobenzene (263 mg, 1.5 mmol) and 2-amino-5-chlorobenzoic acid (86 mg, 0.5 mmol) were reacted according to GP1 to give 6.1 mg of the title compound (166JO85F1). MS (ESI) 347 (MH⁺). Purity for MH⁺ (UV/MS) 100/85.

### Example 3

### 2-Chloro-11-(piperazin-1-yl)-5H-dibenzo[b,e][1,4]diazepine (166JO85F6)

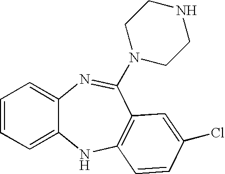

2-Fluoronitrobenzene (212 mg, 1.5 mmol) and 2-amino-5-chlorobenzoic acid (86 mg, 0.5 mmol) were reacted according to GP1 to give 5.3 mg of the title compound (166 JO85F6). MS (ESI) 313 (MH⁺). Purity for MH⁺ (UV/MS) 100/95.

### Example 4

### 2,8-Dichloro-11-(piperazin-1-yl)-5H-dibenzo[b,e][1,4]diazepine (166JO85F2)

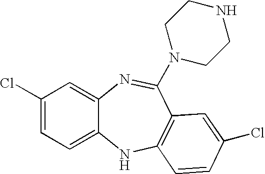

5-Chloro-2-fluoronitrobenzene (263 mg, 1.5 mmol) and 2-amino-5-chlorobenzoic acid (86 mg, 0.5 mmol) were reacted according to GP1 to give 4.8 mg of the title compound (166 JO85F2). MS (ESI) 347 (MH⁺). Purity for MH⁺ (UV/MS) 99/99.

### Example 5

### 8-Bromo-2-chloro-11-(piperazin-1-yl)-5H-dibenzo[b,e][1,4]diazepine (166JO85F3)

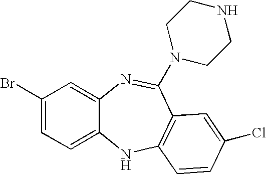

5-Bromo-2-fluoronitrobenzene (330 mg, 1.5 mmol) and 2-amino-5-chlorobenzoic acid (86 mg, 0.5 mmol) were reacted according to GP1 to give 8.0 mg of the title compound (166 JO85F3). MS (ESI) 391 (MH⁺). Purity for MH⁺ (UV/MS) 100/96.

### Example 6

### 2-Chloro-11-(piperazin-1-yl)-8-trifluoromethyl-5H-dibenzo[b,e][1,4]diazepine (166JO85F7)

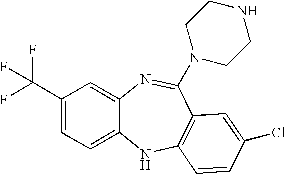

4-Fluoro-3-nitrobenzotrifluoride (314 mg, 1.5 mmol) and 2-amino-5-chlorobenzoic acid (86 mg, 0.5 mmol) were reacted according to GP1 to give 0.3 mg of the title compound (166 JO85F7). MS (ESI) 381 (MH⁺). Purity for MH⁺ (UV/MS) 100/95.

### Example 7

### 6-Chloro-11-(piperazin-1-yl)-8-trifluoromethyl-5H-dibenzo[b,e][1,4]diazepine (189JO77B)

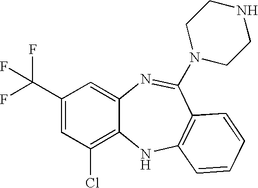

3-Chloro-4-fluoro-5-nitrobenzotrifluoride (366 mg, 1.5 mmol) and 2-aminobenzoic acid (69 mg, 0.5 mmol) were reacted according to GP1 to give 28 mg of the title compound (189JO77B). MS (ESI) 381 (MH⁺). Purity for MH⁺ (UV/MS) 99/100.

### Example 8

### 7-Chloro-11-(piperazin-1-yl)-5H-dibenzo[b,e][1,4]diazepine (160FE35B)

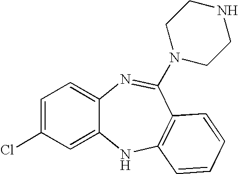

4-Chloro-2-fluoronitrobenzene (528 mg, 3.0 mmol) and 2-aminobenzoic acid (138 mg, 1.0 mmol) were reacted according to GP1 to give 5.0 mg of the title compound (160FE35B). MS (ESI) 313 (MH⁺). Purity for MH⁺ (UV/MS) 99/86.

### Example 9

### 8-Bromo-1-chloro-11-(piperazin-1-yl)-5H-dibenzo[b,e][1,4]diazepine (160FE36A)

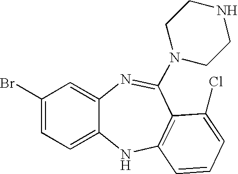

5-Bromo-2-fluoronitrobenzene (660 mg, 3.0 mmol) and 2-amino-6-chlorobenzoic acid (172 mg, 1.0 mmol) were reacted according to GP1 to give 5.0 mg of the title compound (160FE36A). MS (ESI) 391 (MH⁺). Purity for MH⁺ (UV/MS) 94/87.

### Example 10

### 8-Bromo-2-methyl-11-(piperazin-1-yl)-5H-dibenzo[b,e][1,4]diazepine (160FE40C)

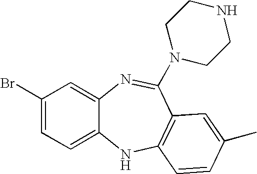

5-Bromo-2-fluoronitrobenzene (660 mg, 3.0 mmol) and 2-amino-5-methylbenzoic acid (152 mg, 1.0 mmol) were reacted according to GP1 to give 7.9 mg of the title compound (160FE40C). MS (ESI) 371 (MH⁺). Purity for MH⁺ (UV/MS) 100/100.

### Example 11

### 4,8-Dichloro-11-(piperazin-1-yl)-5H-dibenzo[b,e][1,4]diazepine (160FE41A)

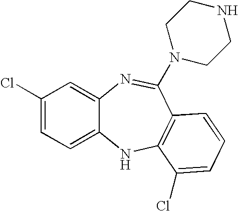

5-Chloro-2-fluoronitrobenzene (527 mg, 3.0 mmol) and 2-amino-3-chlorobenzoic acid (172 mg, 1.0 mmol) were reacted according to GP1 to give 4.6 mg of the title compound (160FE41A). MS (ESI) 347 (MH⁺). Purity for MH⁺ (UV/MS) 95/70.

### Example 12

### 8-Chloro-2-methyl-11-(piperazin-1-yl)-5H-dibenzo[b,e][1,4]diazepine (160FE41B)

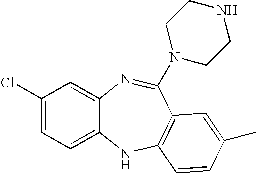

5-Chloro-2-fluoronitrobenzene (527 mg, 3.0 mmol) and 2-amino-5-methylbenzoic acid (151 mg, 1.0 mmol) were reacted according to GP1 to give 7.1 mg of the title compound (160FE41B). MS (ESI) 327 (MH⁺). Purity for MH⁺ (UV/MS) 100/94.

### Example 13

### 8-Chloro-2-fluoro-11-(piperazin-1-yl)-5H-dibenzo[b,e][1,4]diazepine (160FE42A-F3)

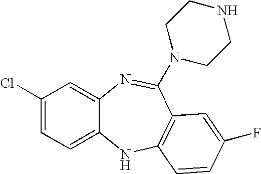

5-Chloro-2-fluoronitrobenzene (264 mg, 1.5 mmol) and 2-amino-5-fluorobenzoic acid (78 mg, 0.5 mmol) were reacted according to GP1 to give 21 mg of the title compound (160FE42A-F3). MS (ESI) 331 (MH⁺). Purity for MH⁺ (UV/MS) 99/98.

### Example 14

### 3,8-Dichloro-11-(piperazin-1-yl)-5H-dibenzo[b,e][1,4]diazepine (160FE42B-F4)

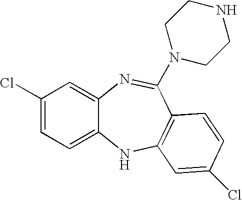

5-Chloro-2-fluoronitrobenzene (264 mg, 1.5 mmol) and 2-amino-4-chlorobenzoic acid (86 mg, 0.5 mmol) were reacted according to GP1 to give 9.4 mg of the title compound (160FE42B-F4). MS (ESI) 347 (MH⁺). Purity for MH⁺ (UV/MS) 99/97.

### Example 15

### 2-Bromo-8-chloro-11-(piperazin-1-yl)-5H-dibenzo[b,e][1,4]diazepine (160FE43A-F6)

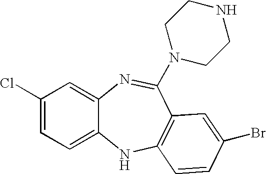

5-Chloro-2-fluoronitrobenzene (528 mg, 3.0 mmol) and 2-amino-5-bromobenzoic acid (216 mg, 1.0 mmol) were reacted according to GP1 to give 20 mg of the title compound (160FE43A-F6). MS (ESI) 391 (MH⁺). Purity for MH⁺ (UV/MS) 100/100.

### Example 16

### 3,7-Dichloro-11-(piperazin-1-yl)-5H-dibenzo[b,e][1,4]diazepine (160FE58D1)

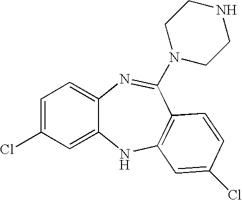

4-Chloro-2-fluoronitrobenzene (263 mg, 1.5 mmol) and 2-amino-4-chlorobenzoic acid (86 mg, 0.5 mmol) were reacted according to GP1 to give 3.1 mg of the title compound (160FE58D1). MS (ESI) 347 (MH⁺). Purity for MH⁺ (UV/MS) 63/83.

### Example 17

### 8-Bromo-3-chloro-11-(piperazin-1-yl)-5H-dibenzo[b,e][1,4]diazepine (160FE58D3)

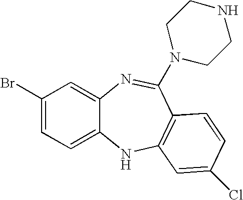

5-Bromo-2-fluoronitrobenzene (330 mg, 1.5 mmol) and 2-amino-4-chlorobenzoic acid (86 mg, 0.5 mmol) were reacted according to GP1 to give 1.1 mg of the title compound (160FE58D3). MS (ESI) 391 (MH⁺). Purity for MH⁺ (UV/MS) 90/85.

### Example 18

### 3-Chloro-11-(piperazin-1-yl)-5H-dibenzo[b,e][1,4]diazepine (160FE58D6)

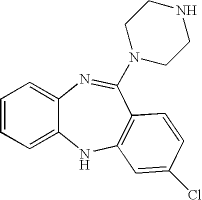

2-Fluoronitrobenzene (212 mg, 1.5 mmol) and 2-amino-4-chlorobenzoic acid (86 mg, 0.5 mmol) were reacted according to GP1 to give 2.2 mg of the title compound (160FE58D6). MS (ESI) 313 (MH⁺). Purity for MH⁺ (UV/MS) 90/100.

### Example 19

### 3-Chloro-11-(piperazin-1-yl)₈-trifluoromethyl-5H-dibenzo[b,e][1,4]diazepine (160FE58D7)

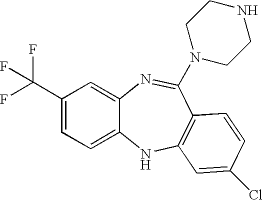

4-Fluoro-3-nitrobenzotrifluoride (314 mg, 1.5 mmol) and 2-amino-4-chlorobenzoic acid (86 mg, 0.5 mmol) were reacted according to GP1 to give 2.0 mg of the title compound (160FE58D7). MS (ESI) 381 (MH⁺). Purity for MH⁺ (UV/MS) 100/100.

### Example 20

### 7-Chloro-2-methyl-11-(piperazin-1-yl)-5H-dibenzo[b,e][1,4]diazepine (160FE58E1)

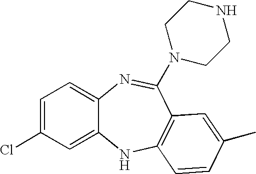

4-Chloro-2-fluoronitrobenzene (263 mg, 1.5 mmol) and 2-amino-5-methylbenzoic acid (76 mg, 0.5 mmol) were reacted according to GP1 to give 1.1 mg of the title compound (160FE58E1). MS (ESI) 327 (MH⁺). Purity for MH⁺ (UV/MS) 100/90.

### Example 21

### 2-Methyl-11-(piperazin-1-yl)-5H-dibenzo[b,e][1,4]diazepine (160FE58E6)

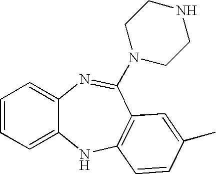

4-Fluoronitrobenzene (212 mg, 1.5 mmol) and 2-amino-5-methylbenzoic acid (76 mg, 0.5 mmol) were reacted according to GP1 to give 6.8 mg of the title compound (160FE58E6). MS (ESI) 293 (MH⁺). Purity for MH⁺ (UV/MS) 100/100.

### Example 22

### 2-Methyl-11-(piperazin-1-yl)-8-trifluoromethyl-5H-dibenzo[b,e][1,4]diazepine (160FE58E7)

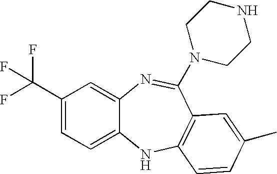

4-Fluoro-3-nitrobenzotrifluoride (314 mg, 1.5 mmol) and 2-amino-5-methylbenzoic acid (76 mg, 0.5 mmol) were reacted according to GP1 to give 1.2 mg of the title compound (160FE58E7). MS (ESI) 361 (MH⁺). Purity for MH⁺ (UV/MS) 100/85.

### Example 23

### 8-Chloro-4-methyl-11-(piperazin-1-yl)-5H-dibenzo[b,e][1,4]diazepine (160FE74C)

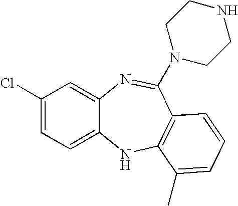

5-Chloro-2-fluoronitrobenzene (1.06 g, 6.0 mmol) and 2-amino-3-methylbenzoic acid (302 mg, 2.0 mmol) were reacted according to GP1 to give 4.8 mg of the title compound (160FE74C). MS (ESI) 327 (MH⁺). Purity for MH⁺ (UV/MS) 97/90.

### Example 24

### 1,8-Dichloro-11-(piperazin-1-yl)-5H-dibenzo[b,e][1,4]diazepine (203FE03)

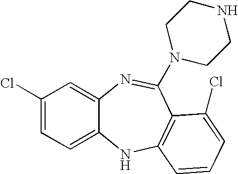

5-Chloro-2-fluoronitrobenzene (1.06 g, 6.0 mmol) and 2-amino-6-chlorobenzoic acid (343 mg, 2.0 mmol) were reacted according to GP1 to give 3.1 mg of the title compound (203FE03). MS (ESI) 347 (MH⁺). Purity for MH⁺ (UV/MS) 100/99.

### Example 25

### 8-Bromo-5-methyl-11-(piperazin-1-yl)-5H-dibenzo[b,e][1,4]diazepine (166JO32)

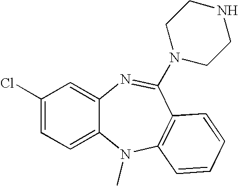

5-Bromo-2-fluoronitrobenzene (580 mg, 2.6 mmol) and N-methylantranilic acid (200 mg, 1.3 mmol) were reacted according to GP1 to give 1.6 mg of the title compound (166JO32). MS (ESI) 371 (MH⁺). Purity for MH⁺ (UV/MS) 90/74.

### Example 26

### General Procedure 2 (GP2)

A mixture of an aminobenzoic acid (1 eq.), a 2-fluoronitrobenezene (3 eq.) or a 2-chloronitrobenzene (3 eq.), and Cs₂CO₃ (3 eq.) in DMF was heated to 140° C. for 1 hour, and then allowed to obtain room temperature. The mixture was diluted with water and washed with EtOAc (2×).

EtOH and Na₂S₂O₄ (5 eq.) was added to the aqueous phase and the resulting mixture was stirred for 1 h. Aqueous HCl (2 M) was added to the mixture and then the aqueous phase was extracted with EtOAc (3×) and the combined organic phases were concentrated.

The residue was taken up in xylene and the resulting mixture was stirred at 130° C. over night. The mixture was diluted with EtOAc, washed with saturated aqueous NaHCO₃-solution, dried (Na₂SO₄), and concentrated.

The residue was taken up in dioxane and added to a mixture of TiCl₄ (1.1 eq., 1 M in toluene) and piperazine (5 eq.) in dioxane at 50° C. The resulting mixture was stirred at 100° C. over night, and then allowed to obtain room temperature. Aqueous HCl (2 M) was added to the mixture until solution became acidic and then the aqueous phase was extracted with EtOAc (2×). Aqueous NaOH (2 M) was added to the aqueous phase until a basic solution was obtained and the resulting suspension was extracted with EtOAc (3×). The combined organic phases were concentrated and purified by HPLC.

### Example 27

### 7,8-Dichloro-11-(piperazin-1-yl)-5H-dibenzo[b,e][1,4]diazepine (166JO28)

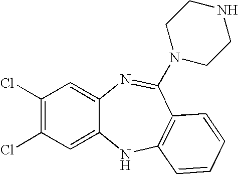

1,2-Dichloro-4-fluoro-5-nitrobenzene (1.26 g, 6.0 mmol) and 2-aminobenzoic acid (274 mg, 2 mmol) were reacted according to GP2 to give 16 mg of the title compound (166JO28). MS (ESI) 347 (MH⁺). Purity for MH⁺ (UV/MS) 99/96.

### Example 28

### 11-(piperazin-1-yl)-8-trifluoromethyl-5H-dibenzo[b,e][1,4]diazepine (166JO23)

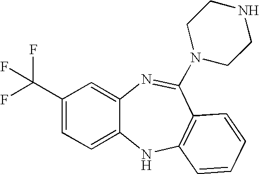

4-Fluoro-3-nitrobenzotrifluoride (1.25 g, 6 mmol) and 2-aminobenzoic acid (274 mg, 2 mmol) were reacted according to GP2 to give 12 mg of the title compound (166JO23). MS (ESI) 347 (MH⁺). Purity for MH⁺ (UV/MS) 81/98.

### Example 29

### 11-(piperazin-1-yl)-5H-dibenzo[b,e][1,4]diazepine (160FE19A)

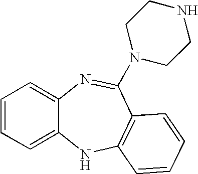

2-Fluoro-nitrobenzene (847 mg, 6.0 mmol) and 2-aminobenzoic acid (274 mg, 2 mmol) were reacted according to GP2 to give 16 mg of the title compound (160FE19A). MS (ESI) 279 (MH⁺). Purity for MH⁺ (UV/MS) 100/100.

### Example 30

### 8-Fluoro-11-(piperazin-1-yl)-5H-dibenzo[b,e][1,4]diazepine (160FE19C)

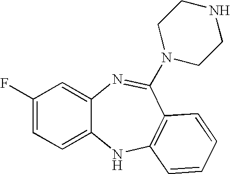

2,5-Difluoronitrobenzene (955 mg, 6.0 mmol) and 2-aminobenzoic acid (274 mg, 2 mmol) were reacted according to GP2 to give 8.9 mg of the title compound (160FE19C). MS (ESI) 297 (MH⁺). Purity for MH⁺ (UV/MS) 99/97.

### Example 31

### 11-(piperazin-1-yl)-5H-dibenzo[b,e][1,4]diazepine-8-carbonitrile (160FE19D)

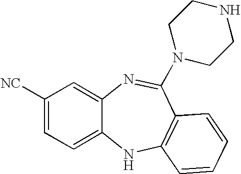

4-Chloro-3-nitrobenzonitrile (1.10 g, 6.0 mmol) and 2-aminobenzoic acid (274 mg, 2 mmol) were reacted according to GP2 to give 4.7 mg of the title compound (160FE19D). MS (ESI) 304 (MH⁺). Purity for MH⁺ (UV/MS) 100/86.

### Example 32

### 8-Bromo-11-(piperazin-1-yl)-5H-dibenzo[b,e][1,4]diazepine (160FE19E)

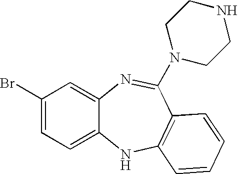

5-Bromo-2-fluoronitrobenzene (1.32 g, 6.0 mmol) and 2-aminobenzoic acid (274 mg, 2 mmol) were reacted according to GP2 to give 15 mg of the title compound (160FE19E). MS (ESI) 357 (MH⁺). Purity for MH⁺ (UV/MS) 100/100.

### Example 33

### 8-Methyl-1-(piperazin-1-yl)-5H-dibenzo[b,e][1,4]diazepine (160FE19F)

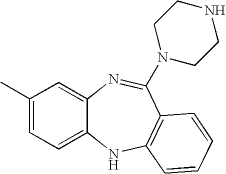

4-Chloro-3-nitrotoluene (1.03 g, 6.0 mmol) and 2-aminobenzoic acid (274 mg, 2 mmol) were reacted according to GP2 to give 1.6 mg of the title compound (160FE19F). MS (ESI) 293 (MH⁺). Purity for MH⁺ (UV/MS) 70/70.

### Example 34

### General Procedure 3 (GP3)

1-chloroethyl chloroformate (17 mg, 0.12 mmol) at 10° C. was added to a N-methyl piperazine derivative (0.1 mmol) dissolved in THF (2 ml). The resulting mixture was then heated at reflux for 18 h. The temperature was decreased and the THF removed at reduced pressure. Methanol was then added to the remaining oil and the mixture was shaken at 65° C. for 2 h. The methanol was removed at reduced pressure and the remaining crude product was purified by HPLC.

### Example 35

### 3-Fluoro-6-piperazin-1-yl-11H-dibenzo[b,e]azepine (160FE02)

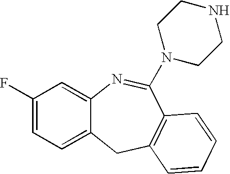

3-Fluoro-6-(4-methyl-piperazin-1-yl)-1H-dibenzo[b,e]azepine (31 mg, 0.1 mmol) was reacted according to GP3 to give 8 mg of the title compound isolated as oxalate salt (160FE02). MS (ESI) 296 (MH⁺). Purity for MH⁺ (UV/MS) 99/100.

### Example 36

### 2-(Trifluoromethanesulfonyloxy)-11-(piperazin-1-yl)-5H-dibenzo[b,e][1,4]diazepine (160FE13A)

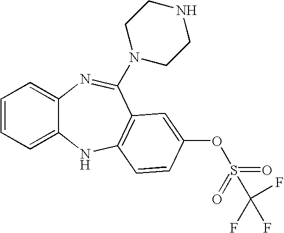

2-(Trifluoromethanesulfonyloxy) 11-(4-methyl-piperazin-1-yl)-5H-dibenzo[b,e][1,4]diazepine (39 mg, 0.1 mmol) was reacted according to GP3 to give 3.0 mg of the title compound (160FE13A). MS (ESI) 427 (MH⁺). Purity for MH⁺ (UV/MS) 95/98.

### Example 37

### 2-(Trifluoromethanesulfonyloxy 11-(piperazin-1-yl)-5H-dibenzo[b,e][1,4]oxazepine (160FE13B)

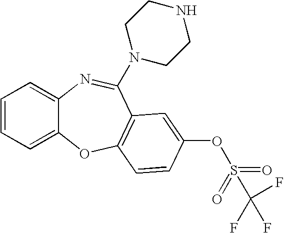

2-(Trifluoromethanesulfonyloxy)-11-(4-methyl-piperazin-1-yl)-5H-dibenzo[b,e][1,4]oxazepine (39 mg, 0.1 mmol) was reacted according to GP3 to give 11 mg of the title compound (160FE13B). MS (ESI) 428 (MH⁺). Purity for MH⁺ (UV/MS) 98/100.

### Example 38

### 8-Chloro-2-(trifluoromethanesulfonyloxy)-11-(piperazin-1-yl)-5H-dibenzo[b,e][1,4]diazepine (160FE13C)

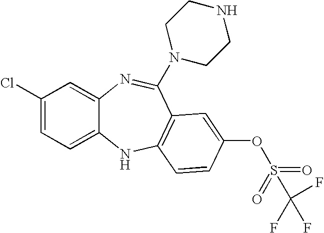

8-Chloro-2-(trifluoromethanesulfonyloxy)-11-(4-methyl-piperazin-1-yl)-5H-dibenzo[b,e][1,4]diazepine (42 mg, 0.1 mmol) was reacted according to GP3 to give 3.2 mg of the title compound (160FE13C). MS (ESI) 461 (MH⁺). Purity for MH⁺ (UV/MS) 100/100.

### Example 39

### 8-(Trifluoromethanesulfonyloxy)-11-(piperazin-1-yl)-5H-dibenzo[b,e][1,4]diazepine (160FE13D)

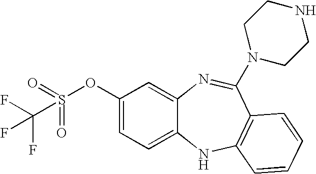

8-(Trifluoromethanesulfonyloxy)-11-(4-methyl-piperazin-1-yl)-5H-dibenzo[b,e][1,4]diazepine (39 mg, 0.1 mmol) was reacted according to GP3 to give 2.2 mg of the title compound (160FE13D). MS (ESI) 427 (MH⁺). Purity for MH⁺ (UV/MS) 100/100.

### Example 40

### General Procedure 4 (GP4)

A mixture of appropriate lactam (0.1 mmol) in dioxane was added to a mixture of TiCl₄ (1.1 eq., 1 M in toluene) and the amine (0.5 mmol) in dioxane at 50° C. or to a mixture of TiCl₄ (2.2 eq., 1 M in toluene) and the amine (1.0 mmol) in dioxane at 50° C. The resulting mixture was stirred at 100° C. over night, and then allowed to obtain room temperature. Aqueous HCl (3 mL, 2 M) was added to the aqueous mixture and then the aqueous phase was extracted with EtOAc (2×4 mL). Aqueous NaOH (6 mL, 2 M) was added to the aqueous phase and the resulting suspension was extracted with EtOAc (3×3 mL). The combined organic phases were concentrated and purified by HPLC.

### Example 41

### 11-(piperazin-1-yl)-dibenzo[b,f][1,4]thiazepin (160FE17A)

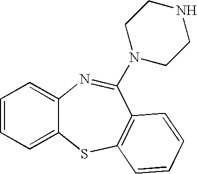

10H-Dibenzo[b,f][1,4]thiazepin-11-one (23 mg, 0.1 mmol) and piperazine (43 mg, 0.5 mmol) were reacted according to GP4 to give 3.1 mg of the title compound (160FE17A). MS (ESI) 296 (MH⁺). Purity for MH⁺ (UV/MS) 97/90.

### Example 42

### 11-(piperazin-1-yl)-2,3-dihydro-1,4-benzodioxino[6,7-b][1,4]benzothiazepin (160FE17B)

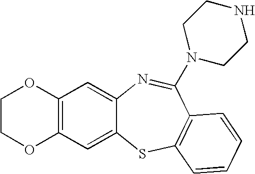

2,3-Dihydro-1,4-benzodioxino[6,7-b][1,4]benzothiazepin-11(12H)-one (29 mg, 0.1 mmol) and piperazine (43 mg, 0.5 mmol) were reacted according to GP4 to give 1.9 mg of the title compound (160FE17B). MS (ESI) 354 (MH⁺). Purity for MH⁺ (UV/MS) 99/95.

### Example 43

### 8-Chloro-11-[1,4]diazepam-1-yl-5H-dibenzo[b,e][1,4]diazepine (160FE16A)

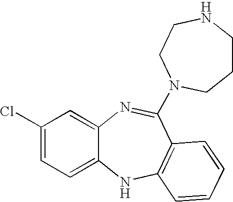

8-Chloro-5,10-dihydro-dibenzo[b,e][1,4]diazepine-11-one (25 mg, 0.1 mmol) and homopiperazine (50 mg, 0.5 mmol) were reacted according to GP4 to give 12 mg of the title compound (160FE16A). MS (ESI) 327 (MH⁺). Purity for MH⁺ (UV/MS) 99/93.

### Example 44

### N′-(8-Chloro-5H-dibenzo[b,e][1,4]diazepine-11-yl)-N,N-dimethyl-ethane-1,2-diamine (160FE16D)

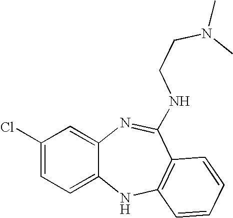

8-Chloro-5,10-dihydro-dibenzo[b,e][1,4]diazepine-11-one (25 mg, 0.1 mmol) and N,N-dimethylethylenediamine (44 mg, 0.5 mmol) were reacted according to GP4 to give 20 mg of the title compound (160FE16D). MS (ESI) 315 (MH⁺). Purity for MH⁺ (UV/MS) 100/100.

### Example 45

### N′-(8-Chloro-5H-dibenzo[b,e][1,4]diazepine-11-yl)-N N-diethyl-ethane-1,2-diamine (160FE16E)

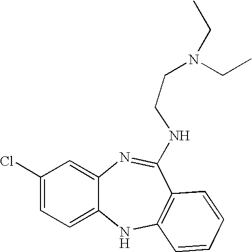

8-Chloro-5,10-dihydro-dibenzo[b,e][1,4]diazepine-11-one (25 mg, 0.1 mmol) and N,N-diethylethylenediamine (58 mg, 0.5 mmol) were reacted according to GP4 to give 3.9 mg of the title compound (160FE16E). MS (ESI) 343 (MH⁺). Purity for MH⁺ (UV/MS) 99/94.

### Example 46

### 8-Chloro-11-(4-methyl-[1,4]diazepam-1-yl)-5H-dibenzo[b,e][1,4]diazepine (160FE16F)

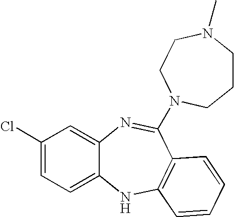

8-Chloro-5,10-dihydro-dibenzo[b,e][1,4]diazepine-11-one (25 mg, 0.1 mmol) and 1-methylhomopiperazine (57 mg, 0.5 mmol) were reacted according to GP4 to give 5.7 mg of the title compound (160FE16F). MS (ESI) 341 (MH⁺). Purity for MH⁺ (UV/MS) 100/100.

### Example 47

### 8-Chloro-2-methoxy-11-(piperazin-1-yl)-5H-dibenzo[b,e][1,4]diazepine (160FE20A)

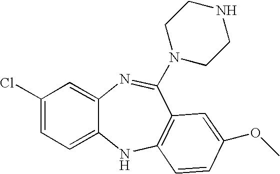

8-Chloro-2-methoxy-5,10-dihydro-dibenzo[b,e][1,4]diazepine-11-one (28 mg, 0.1 mmol) and piperazine (86 mg, 1.0 mmol) were reacted according to GP4 to give 19 mg of the title compound (160FE20A). MS (ESI) 342 (MH⁺). Purity for MH⁺ (UV/MS) 99/100.

### Example 48

### N-(5H-Dibenzo[b,e][1,4]diazepine-11-yl)-N,N-dimethyl-ethane-1,2-diamine (160FE20B)

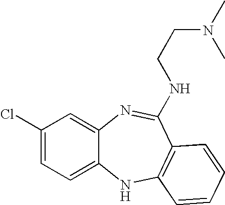

5,10-Dihydro-dibenzo[b,e][1,4]diazepine-11-one (160FE15A) (21 mg, 0.1 mmol) and N,N-dimethylethylenediamine (88 mg, 1.0 mmol) were reacted according to GP4 to give 7.6 mg of the title compound (160FE20B). MS (ESI) 281 (MH⁺). Purity for MH⁺ (UV/MS) 100/100.

### Example 49

### 11-[1,4]Diazepam-1-yl-5H-dibenzo[b,e][1,4]diazepine (160FE20C)

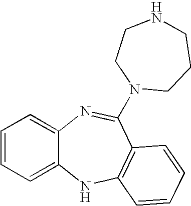

5,10-Dihydro-dibenzo[b,e][1,4]diazepine-11-one (160FE15A) (21 mg, 0.1 mmol) and homopiperazine (100 mg, 1.0 mmol) were reacted according to GP4 to give 12 mg of the title compound (160FE20C). MS (ESI) 293 (MH⁺). Purity for MH⁺ (UV/MS) 95/95.

### Example 50

### N′-(8-Fluoro-5H-dibenzo[b,e][1,4]diazepine-11-yl)-N,N-dimethyl-ethane-1,2-diamine (160FE20D)

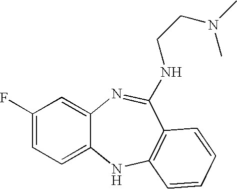

8-Fluoro-5,10-dihydro-dibenzo[b,e][1,4]diazepine-11-one (160FE15C) (23 mg, 0.1 mmol) and N,N-dimethylethylenediamine (88 mg, 1.0 mmol) were reacted according to GP4 to give 11 mg of the title compound (160FE20D). MS (ESI) 299 (MH⁺). Purity for MH⁺ (UV/MS) 100/100.

### Example 51

### 8-Fluoro-11-[1,4]diazepam-1-yl-5H-dibenzo[b,e][1,4]diazepine (160FE16A)

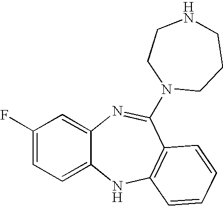

8-Fluoro-5,10-Dihydro-dibenzo[b,e][1,4]diazepine-11-one (160FE15C) (23 mg, 0.1 mmol) and homopiperazine (100 mg, 1.0 mmol) were reacted according to GP4 to give 19 mg of the title compound (160FE20E). MS (ESI) 311 (MH⁺). Purity for MH⁺ (UV/MS) 100/100.

### Example 52

### N′-(8-Chloro-5H-dibenzo[b,e][1,4]diazepine-11-yl)-N-methyl-ethane-1,2-diamine (160FE22)

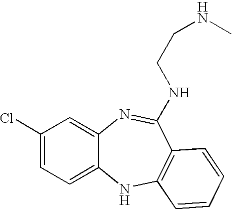

8-Chloro-5,10-dihydro-dibenzo[b,e][1,4]diazepine-11-one (25 mg, 0.1 mmol) and N-methylethylenediamine (74 mg, 1.0 mmol) were reacted according to GP4 to give 7.6 mg of the title compound (160FE22). MS (ESI) 301 (MH⁺). Purity for MH⁺ (UV/MS) 92/83.

### Example 53

### 8-Chloro-11-(trans-2,5-dimethyl-piperazin-1-yl)-5H-dibenzo[b,e][1,4]diazepine (160FE33A)

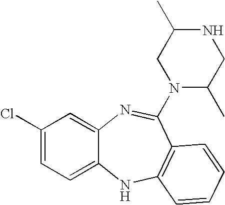

8-Chloro-5,10-dihydro-dibenzo[b,e][1,4]diazepine-11-one (25 mg, 0.1 mmol) and trans-2,5-dimethylpiperazine (114 mg, 1.0 mmol) were reacted according to GP4 to give 1.9 mg of the title compound (160FE33A). MS (ESI) 341 (MH⁺). Purity for MH⁺ (UV/MS) 100/82.

### Example 54

### 8-Chloro-11-(3,5-dimethyl-piperazin-1-yl)-5H-dibenzo[b,e][1,4]diazepine (160FE33B)

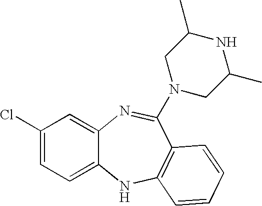

8-Chloro-5,10-dihydro-dibenzo[b,e][1,4]diazepine-11-one (25 mg, 0.1 mmol) and 2,6-dimethylpiperazine (114 mg, 1.0 mmol) were reacted according to GP4 to give 18 mg of the title compound (160FE33B). MS (ESI) 341 (MH⁺). Purity for MH⁺ (UV/MS) 100/100.

### Example 55

### 8-Chloro-11-(3-methyl-piperazin-1-yl)-5H-dibenzo[b,e][1,4]diazepine (160FE38)

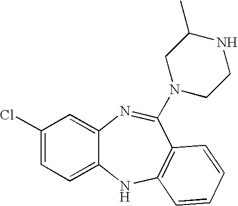

8-Chloro-5,10-dihydro-dibenzo[b,e][1,4]diazepine-11-one (25 mg, 0.1 mmol) and 2-methylpiperazine (100 mg, 1.0 mmol) were reacted according to GP4 to give 30 mg of the title compound (160FE38). MS (ESI) 327 (MH⁺). Purity for MH⁺ (UV/MS) 100/89.

### Example 56

### 8-Chloro-11-(3-phenyl-piperazin-1-yl)-5H-dibenzo[b,e][1,4]diazepine (160FE45)

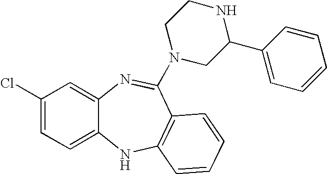

8-Chloro-5,10-dihydro-dibenzo[b,e][1,4]diazepine-11-one (25 mg, 0.1 mmol) and 2-phenylpiperazine (162 mg, 1.0 mmol) were reacted according to GP4 to give 27 mg of the title compound (160FE45). MS (ESI) 389 (MH⁺). Purity for MH⁺ (UV/MS) 100/89.

### Example 57

### 8-Chloro-5-methyl-11-(piperazin-1-yl)-5H-dibenzo[b,e][1,4]diazepine (189JO25A)

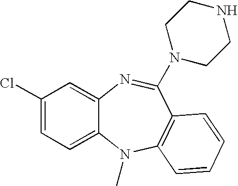

NaH (12 mg, 0.29 mmol, 60% in mineral oil) was added to a mixture of 8,5-dichloro-5H-dibenzo[b,e][1,4]diazepine (160FE64) (50 mg, 0.19 mmol) in toluene (1.5 mL) and DMF (0.5 mL). MeI (24 μL, 0.38 mmol) was then added. The resulting mixture was stirred for 1 h then quenched by addition of saturated aqueous NaHCO₃-solution (2 mL). The mixture was extracted with diethyl ether, and the combined organic phases were dried (Na₂SO₄) and concentrated. The residue was taken up in toluene (2.0 mL), piperazine (98 mg, 1.1 mmol) was added, and the resulting mixture was stirred at 100° C. for 1 h. Aqueous HCl (1 mL, 2M) and EtOAc (2 mL) was then added to the mixture. The phases were separated and the aqueous phase was extracted with EtOAc (2 mL) and then aqueous NaOH (2 mL, 2 M) was added. The basic aqueous phase was extracted with EtOAc (3×2 mL) and the combined organic phases were dried (Na₂SO₄) and concentrated. The residue was dissolved in DMF and purified on HPLC to give 34 mg of the title compound (189JO25A). MS (ESI) 327 (MH⁺). Purity for MH⁺ (UV/MS) 100/100.

### Example 58

### 8-Chloro-5-benzyl-11-(piperazin-1-yl)-5H-dibenzo[b,e][1,4]diazepine (160FE46-PIPBN)

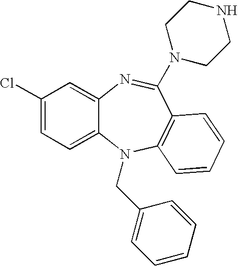

8,5-Dichloro-5H-dibenzo[b,e][1,4]diazepine (160FE46) (51 mg, 0.20 mmol) and benzyl bromide (68 mg, 0.4 mmol) were reacted as described for Example 57 to give 8.4 mg of the title compound (160FE46-PIPBN). MS (ESI) 403 (MH⁺). Purity for MH⁺ (UV/MS) 100/100.

### Example 59

### 8-Iodo-11-(piperazin-1-yl)-5H-dibenzo[b,e][1,4]diazepine (166JO38)

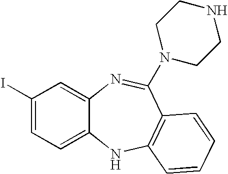

A mixture of 8-bromo-5,10-dihydro-dibenzo[b,e][1,4]diazepine-11-one (166JO31) (60 mg, 0.21 mmol), NaI, (62 mg, 0.42 mmol), N,N-dimethylethylenediamine (2.2 μL, 0.021 mmol) and CuI (2 mg, 0.01 mmol) in dioxane (1 ml) was heated in a capped tube for 3 days. The reaction mixture was allowed to obtain room temperature and then the mixture was applied onto a SCX-2 ion exchange column and the product was eluted with CH₂Cl₂ to give 49 mg of intermediate 8-iodolactam. The intermediate 8-iodolactam (20 mg, 0.060 mmol) in dioxane (1 mL) was added to a mixture of TiCl₄ (0.13 mL, 0.13 mmol, 1 M in toluene) and piperazine (0.051 g, 0.60 mmol) in dioxane at 50° C. The resulting mixture was stirred at 100° C. over night then allowed to obtain room temperature. Aqueous HCl (3 mL, 2 M) was added to the mixture and then the aqueous phase was extracted with EtOAc (2×4 mL). Aqueous NaOH (6 mL, 2 M) was added to the aqueous phase and the resulting suspension was extracted with EtOAc (3×3 mL). The combined organic phases were concentrated and purified by HPLC to give 4.1 mg of the title compound (166JO38). MS (ESI) 405 (MH⁺). Purity for MH⁺ (UV/MS) 100/100.

### Example 60

### 2-Iodo-8-chloro-11-(piperazin-1-yl)-5H-dibenzo[b,e][1,4]diazepine (166JO54)

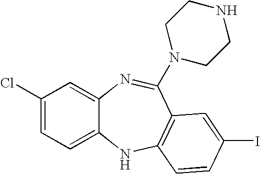

2-Bromo-8-chloro-5,10-dihydro-dibenzo[b,e][1,4]diazepine-11-one (intermediate from GP1) (30 mg, 0.09 mmol) was reacted as described for Example 59 to give 7.0 mg of the title compound (166JO54). MS (ESI) 439 (MH⁺). Purity for MH⁺ (UV/MS) 100/100.

### Example 61

### 8-Phenyl-11-(piperazin-1-yl)-5H-dibenzo[b,e][1,4]diazepine (189JO53)

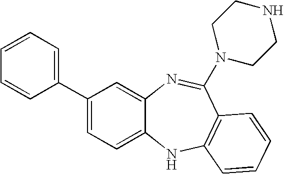

Tetrakis(triphenylphosphine)palladium(0) (catalytic amount) was added to a mixture of 8-bromo-5,10-dihydro-dibenzo[b,e][1,4]diazepine-11-one (166JO31) (30 mg, 0.12 mmol), benzene boronic acid (18 mg, 0.15 mmol) and K₂CO₃ (34 mg, 0.24 mmol) in deoxygenised toluene/EtOH/H₂O (1.5 mL) and the resulting mixture was stirred at 80° C. over night. The mixture was diluted with EtOAc, washed with saturated aqueous NaHCO₃-solution, dried (Na₂SO₄) and concentrated to give crude 8-phenyl lactam. The intermediate 8-phenyl lactam in dioxane (1 mL) was added to a mixture of TiCl₄ (0.24 mL, 0.24 mmol, 1 M in toluene) and piperazine (0.103 g, 1.2 mmol) in dioxane at 50° C. The resulting mixture was stirred at 100° C. over night, and then allowed to obtain room temperature. Aqueous HCl (3 mL, 2 M) was added to the mixture and then the aqueous phase was extracted with EtOAc (2×4 mL). Aqueous NaOH (6 mL, 2 M) was added to the aqueous phase and the resulting suspension was extracted with EtOAc (3×3 mL). The combined organic phases were applied onto a SCX-2 ion exchange column. The column was washed with MeOH, and then the product was eluted with NH₃ (7 N in MeOH), concentrated, and purified by HPLC to give 16 mg of the title compound (189JO53). MS (ESI) 355 (MH⁺). Purity for MH⁺ (UV/MS) 100/100.

### Example 62

### 8-Chloro-11-(piperidin-1-yl)-5H-dibenzo[b,e][1,4]diazepine (166JO69A)

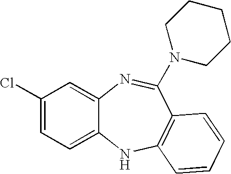

Piperidine (37 mg, 0.44 mmol) was added to crude 8-chloro-11-methylsulfanyl-5H-dibenzo[b,e][1,4]diazepine (166JO50) (90 mg, purity 50%, 0.218 mmol) in pyridine (2 mL) and the resulting mixture was heated in a capped tube at 160° C. for 10 h. The mixture was concentrated and flash chromatographed (SiO₂, heptane:EtOAc 8:1-6:1) to give 12 mg of the title compound (166JO69A). MS (ESI) 312 (MH⁺). Purity for MH⁺ (UV/MS) 100/100.

### Example 63

### 8-Chloro-11-(morpholin-4-yl)-5H-dibenzo[b,e][1,4]diazepine (166JO69B)

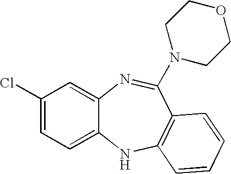

Crude 8-chloro-11-methylsulfanyl-5H-dibenzo[b,e][1,4]diazepine (166JO50) (90 mg, purity 50%, 0.218 mmol) and morpholine (38 mg, 0.44 mmol) were reacted as described for Example 62 to give 11 mg of the title compound (166JO69B). MS (ESI) 314 (MH⁺). Purity for MH⁺ (UV/MS) 100/98.

### Example 64

### 5-Allyl-8-chloro-11-(piperazin-1-yl)-5H-dibenzo[b,e][1,4]diazepine (166JO68)

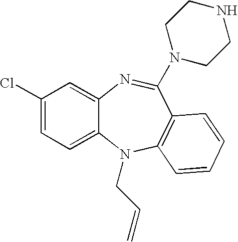

K^{t}OBu (343 mg, 3.1 mmol) was added to a mixture of 8-chloro-5,10-dihydro-dibenzo[b,e][1,4]diazepine-11-one (500 mg, 2.0 mmol) in dioxane (10 mL) and the resulting mixture was stirred at 60° C. for 1 h, then cooled to room temperature. p-Methoxybenzyl chloride (0.42 mL, 3.1 mmol) was added and the resulting mixture was stirred at 40° C. for 2 h. The reaction was quenched by addition of MeOH (2 mL). The mixture was diluted with CH₂Cl₂, washed with saturated aqueous NaHCO₃-solution, dried (Na₂SO₄), concentrated and flash chromatographed (SiO₂, heptane:EtOAc, 4:1-3:1) which gave intermediate p-methoxybenzyl protected lactam (732 mg), 85% pure, which was used in the next step without further purification.

To a mixture of p-methoxybenzylprotected lactam (100 mg, 0.27 mmol) in DMF (2 mL) was added NaH (16 mg, 0.41 mmol, 60% in mineral oil) and the resulting mixture was heated to 60° C. then allowed to obtain room temperature. Allyl bromide (36 μL, 0.41 mmol) was added and the resulting mixture was stirred at room temperature for 3 h then diluted with CH₂Cl₂, washed with saturated aqueous NaHCO₃-solution, dried (Na₂SO₄), concentrated, flash chromatographed (SiO₂, heptane:EtOAc 8:1-4:1), and concentrated. The residue was taken up in trifluoroacetic acid (4 mL) and the resulting mixture was stirred at room temperature over night, then at 45° C. for 2 h. The mixture was concentrated, chromatographed (SiO₂, heptane:EtOAc 8:1-4:1), and concentrated. The residue was taken up in toluene (2 mL) and N,N-dimethylaniline (48 μL, 0.38 mmol) and POCl₃ (35 μL, 0.38 mmol) were added. The resulting mixture was stirred at 100° C. for 2 h then concentrated. The residue was taken up in dioxane, piperazine (65 mg, 0.76 mmol) was added and the resulting mixture was stirred at 100° C. for 3 h. To the mixture was added aqueous HCl (3 mL, 2 M) and then the aqueous phase was extracted with EtOAc (2×4 mL). To the aqueous phase was added aqueous NaOH (6 mL, 2 M) and the resulting suspension was extracted with EtOAc (3×3 mL). The combined organic phases were concentrated and purified by HPLC to give 17 mg of the title compound (166JO68). MS (ESI) 353 (MH⁺). Purity for MH⁺ (UV/MS) 99/88.

### Example 65

### 6-Chloro-11-(piperazin-1-yl)-5H-dibenzo[b,e][1,4]diazepine (189JO68)

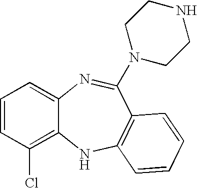

A mixture of a methyl 2-aminobenzoate (454 mg, 3.0 mmol), 3-chloro-2-fluoronitrobenezene (352 mg, 2 mmol) and Cs₂CO₃ (0.78 g, 2.4 mol) in DMF (4 mL) was stirred at 140° C. for 2 h.

The mixture was diluted with EtOAc (10 mL) and washed with 2 M aqueous NaOH-solution (2×5 mL), dried (Na₂SO₄), concentrated and flash chromatographed (SiO₂, toluene:heptane:EtOAc-system) and concentrated. The residue was taken up in THF (10 mL), 1 M aqueous LiOH (5 mL) was added and the resulting mixture was stirred at 80° C. for 1 h, and then allowed to obtain room temperature. 2 M aqueous HCl was added until pH 2. The aqueous phase was extracted with EtOAc (3×). The combined organic phases were dried (Na₂SO₄) and concentrated. The residue was taken up in EtOH and a mixture of K₂CO₃ (1.38 g, 10 mmol) and Na₂S₂O₄ (1.74 g, 10 mmol) in water was added and the resulting mixture was stirred for 1 h. The mixture was diluted with water and washed with 1 M aqueous NaOH-solution (2×5 mL) and then dried (Na₂SO₄) and concentrated.

The residue was taken up in CH₂Cl₂ and 1-ethyl-3-(3-dimethylaminopropyl)carbodiimide hydrochloride (307 mg, 1.6 mmol) was added. The resulting mixture was stirred at room temperature for 1 h. The mixture was diluted with EtOAc, washed with saturated aqueous NaHCO₃-solution, dried (Na₂SO₄), concentrated, and flash chromatographed (SiO₂, heptane:EtOAc, 2:1) to give 21 mg of the intermediate lactam.

The intermediate lactam was taken up in dioxane and added to a mixture of TiCl₄ (0.19 mL, 0.19 mmol, 1 M in toluene) and piperazine (73 mg, 0.85 mmol) in dioxane at 50° C. The resulting mixture was stirred at 100° C. over night, and then allowed to obtain room temperature. To the mixture was added aqueous HCl (1 mL, 2 M) and then the aqueous phase was extracted with EtOAc (2×1 mL). To the aqueous phase was added aqueous NaOH (2 mL, 2 M) and the resulting suspension was extracted with EtOAc (3×1 mL). The combined organic phases were concentrated and purified by HPLC to give 9.8 mg of the title compound (189JO68) MS (ESI) 313 (MH⁺). Purity for MH⁺ (UV/MS) 100/98.

### Example 66

### 8-Chloro-5-piperazin-1-yl-1H-benzo[b]pyrido[2,3-e][1,4]diazepine (166JO63)

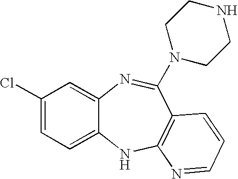

To a mixture of 5-chloro-2-nitroaniline (345 mg, 2 mmol) and pyridine (162 μL, 2 mmol) in dioxane was added 2-chloronicotinyl chloride (352 mg, 2 mmol) and the resulting mixture was stirred at room temperature for 2 h. The mixture was diluted with CH₂Cl₂, washed with saturated aqueous NaHCO₃-solution, dried (Na₂SO₄), concentrated and crystallised from MeOH to give 271 mg of intermediate diarylamine. To a mixture of intermediate diarylamine (100 mg, 0.32 mmol) in EtOH (0.5 mL) was added a mixture of K₂CO₃ (220 mg, 1.6 mmol) and Na₂S₂O₄ (278 mg, 1.6 mmol) in water (0.5 mL) and the resulting mixture was stirred for 1 h at room temperature. The mixture was concentrated and the residue taken up in EtOAc/H₂O and separated. The organic phase was dried (Na₂SO₄) and concentrated. The residue was taken up in xylene and heated to 130° C. over night, then diluted with EtOAc, washed with saturated aqueous NaHCO₃-solution, dried (Na₂SO₄), concentrated and flash chromatographed (SiO₂, heptane:EtOAc) to give intermediate lactam. The intermediate lactam was taken up in dioxane and added to a mixture of TiCl₄ (187 μL, 0.187 mmol, 1 M in toluene) and piperazine (73 mg, 0.85 mmol) in dioxane at 50° C. The resulting mixture was stirred at 100° C. over night, and then allowed to obtain room temperature. To the mixture was added aqueous HCl (1 mL, 2 M) and then the aqueous phase was extracted with EtOAc (2×2 mL). To the aqueous phase was added aqueous NaOH (2 mL, 2 M) and the resulting suspension was extracted with EtOAc (3×1 mL). The combined organic phases were concentrated and purified by HPLC to give 20 mg of the title compound (166JO63). MS (ESI) 314 (MH⁺). Purity for MH⁺ (UV/MS) 100/99.

### Example 67

### 2-Chloro-10-piperazin-1-yl-5H-dibenzo[b,f]azepin (189JO39)

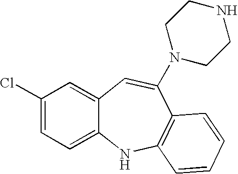

To a mixture under Ar of 2-chloro-5-(4-methoxybenzyl)-5,11-dihydrodibenzo[b,f]azepin-11-one (189JO27) (150 mg, 0.41 mmol) in CH₂Cl₂ (10 mL) at −75° C. was added TiCl₄ (0.60 mL, 0.60 mmol, 1 M in toluene) and the resulting mixture was stirred for 1 h. The mixture was diluted with saturated aqueous NH₄Cl-solution and CH₂Cl₂ and the mixture was allowed to obtain room temperature and the phases were separated. The aqueous phase was extracted with CH₂Cl₂ (1×10 mL) and the combined organic phases were dried (Na₂SO₄) and concentrated to give crude protected product (90 mg, 90%), that was used in the next step without further purification.

To a solution of TiCl₄ (0.18 mL, 0.18 mmol, 1 M in toluene) and piperazine (283 mg, 3.3 mmol) in dioxane (4 mL) at 50° was added crude protected product (80 mg, 0.33 mmol) and the resulting suspension was stirred at 100° C. for 1.5 h. The mixture was allowed to obtain room temperature, then it was diluted with EtOAc, washed with saturated aqueous NaHCO₃-solution, dried (Na₂SO₄), concentrated and flash chromatographed (Al₂O₃, CH₂Cl₂:MeOH, 1:0-25:1) to give 64 mg (63%) of the title compound (189JO39). MS (ESI) 312 (MH⁺). Purity for MH⁺ (UV/MS) 97/95.

### Example 68

### 8-Chloro-11-(piperazin-1-yl)-dibenzo[b,f][1,4]thiazepine (189JO16)

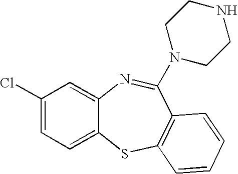

To a mixture of 8-chloro-10H-dibenzo[b,f][1,4]thiazepin-11-one (189JO13) (38 mg, 0.15 mmol) and N,N-dimethylaniline (46 μL, 0.36 mmol) in toluene was added POCl₃ (27 μL, 0.29 mmol) and the resulting mixture was stirred for 2 h at 100° C., and then concentrated. Toluene (2 mL) and piperazine (62 mg, 0.73 mmol) were added, and the resulting mixture was stirred at 100° C. for 3 h, and then allowed to obtain room temperature. To the mixture was added aqueous HCl (1 mL, 2 M) and then the aqueous phase was extracted with EtOAc (2×2 mL). To the aqueous phase was added aqueous NaOH (3 mL, 2 M) and the resulting mixture was extracted with EtOAc (3×3 mL). The combined organic phases were concentrated and purified by HPLC to give 6.6 mg of the title compound (189JO16). MS (ESI) 330 (MH⁺). Purity for MH⁺ (UV/MS) 99/98.

### Example 68

### 8-Chloro-11-(piperazin-1-yl)-dibenzo[b,f][1,4]oxazepine (189JO31)

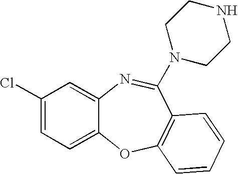

A mixture of 8-chloro-10H-dibenzo[b,f][1,4]oxazepin-11-one (189JO29C) (17 mg, 0.069 mmol) and 2,4-bis(4-methoxyphenyl)-1,3-dithia-2,4-diphosphetane-2,4-disulfide (16 mg, 0.040 mmol) in toluene (2 mL) was heated in capped tube using microwave assisted heating (130° C., 20 minutes). The reaction mixture was cooled to room temperature and MeI (18 μL, 0.29 mmol) was added and the resulting mixture was heated in capped tube using microwave assisted heating (120° C., 20 minutes). The mixture was concentrated and the residue was taken up in pyridine (2 mL) and piperazine (25 mg, 0.29 mmol) was added. The resulting mixture was heated in a capped tube at 130° C. over night then using microwave assisted heating (160° C., 30 minutes). The mixture was concentrated, diluted with EtOAc and washed with water. The organic phase was applied onto a SCX-2 ion exchange column. The column was washed with MeOH, and then the product was eluted with NH₃ (7 N in MeOH) to give 9.0 mg (57%) of the title compound (189JO31). MS (ESI) 314 (MH⁺). Purity for MH⁺ (UV/MS) 92/100.

### Example 69

### 8-Chloro-11-(4-methyl-piperazin-1-yl)-dibenzo[b,f][1,4]oxazepine (189JO47)

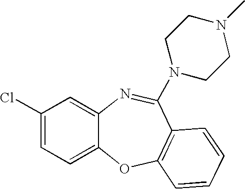

A mixture of 8-chloro-10H-dibenzo[b,f][1,4]oxazepin-11-one (189JO29C) (30 mg, 0.069 mmol) and 2,4-bis(4-methoxyphenyl)-1,3-dithia-2,4-diphosphetane-2,4-disulfide (29 mg, 0.040 mmol) in toluene (2 mL) was heated in a capped tube using microwave assisted heating (130° C., 20 minutes). The reaction mixture was cooled to room temperature and MeI (38 μL, 0.29 mmol) was added and the resulting mixture was heated in capped tube using microwave assisted heating (120° C., 20 minutes). The mixture was concentrated and the residue was taken up in pyridine (2 mL) and piperazine (24 mg, 0.29 mmol) was added. The resulting mixture was heated in a capped tube at 130° C. over night then heated using microwave assisted heating (160° C., 30 minutes). The mixture was concentrated, diluted with EtOAc and washed with water. The organic phase was dried (Na₂SO₄), concentrated and flash chromatographed (SiO₂, toluene:EtOAc:MeOH, 4:2:0-2:2:1) to give 8.9 mg of the title compound (189JO47). MS (ESI) 328 (MH⁺). Purity for MH⁺ (UV/MS) 98/93.

### Example 70

### 3-Chloro-6-piperazin-1-yl-11H-dibenzo[b,e]azepine (189JO60)

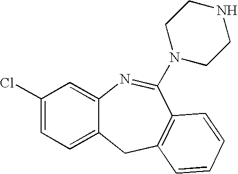

3-Chloro-5,11-dihydro-dibenzo[b,e]azepin-6-one (189JO59) (25 mg, 0.1 mmol) and piperazine were reacted according to GP4 to give 2.2 mg of the title compound (189JO60). MS (ESI) 312 (MH⁺). Purity for MH⁺ (UV/MS) 100/100.

### Example 71

### General Procedure 5 (GP5)

A mixture of a methyl aminobenzoic ester (2.0 mmol), a 2-fluoronitrobenezene (1.0 mmol) and Cs₂CO₃ (0.65 g, 2.0 mmol) in DMF (4 mL) was stirred at 40° C. for 2 h. The mixture was diluted with EtOAc (10 mL) and washed with 2 M aqueous NaOH-solution (2×5 mL).

EtOH, H₂O, K₂CO₃ (0.69 g, 5 mmol) and Na₂S₂O₄ (0.87 g, 5 mmol) was added to the EtOAc-phase and the resulting mixture was stirred vigorously for 1 h. The aqueous phase was removed and the organic phase was washed with 1 M aqueous NaOH-solution (2×5 mL) and then concentrated.

The residue was taken up in DMF (1 mL), toluene (4 mL) and NaH (60 mg, 1.5 mmol, 60% in mineral oil) was added and the resulting mixture was stirred at 80° C. over night, then quenched by addition of saturated aqueous NH₄Cl-solution. The resulting mixture was diluted with EtOAc, washed with 2 M aqueous NaOH-solution (2×5 mL), dried (Na₂SO₄) and concentrated. The residue was taken up in dioxane and added to a mixture of TiCl₄ (1.1 mL, 1.1 mmol, 1 M in toluene) and piperazine (0.41 g, 5 mmol) in dioxane at 50° C. The resulting mixture was stirred at 100° C. over night, and then allowed to obtain room temperature. To the mixture was added aqueous HCl (3 mL, 2 M) and then the aqueous phase was extracted with EtOAc (2×4 mL). To the aqueous phase was added aqueous NaOH (6 mL, 2 M) and the resulting suspension was extracted with EtOAc (3×3 mL). The combined organic phases were concentrated, dried (Na₂SO₄) and purified by HPLC.

### Example 72

### 8-Bromo-1-(piperazin-1-yl)-dibenzo[b,f][1,4]oxazepine (189JO48A)

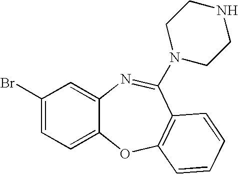

5-Bromo-2-fluoronitrobenzene (220 mg, 1 mmol) and methyl 2-hydroxybenzoate (304 mg, 2 mmol) were reacted according to GP5 to give 36 mg of the title compound (189JO48A). MS (ESI) 358 (MH⁺). Purity for MH⁺ (UV/MS) 96/82.

### Example 73

### 11-(piperazin-1-yl)-dibenzo[b,f][1,4]oxazepine (189JO48B)

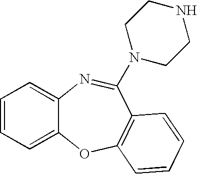

2-fluoronitrobenzene (141 mg, 1 mmol) and methyl 2-hydroxybenzoate (304 mg, 2 mmol) were reacted according to GP5 to give 5.2 mg of the title compound (189JO48B). MS (ESI) 280 (MH⁺). Purity for MH⁺ (UV/MS) 99/99.

### Example 74

### 7-Chloro-11-(piperazin-1-yl)-dibenzo[b,f][1,4]oxazepine (189JO50A)

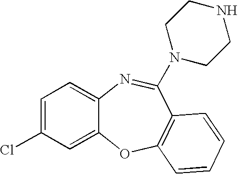

4-Chloro-2-fluoronitrobenzene (175 mg, 1 mmol) and methyl 2-hydroxybenzoate (304 mg, 2 mmol) were reacted according to GP5 to give 17 mg of the title compound (189JO50A). MS (ESI) 314 (MH⁺). Purity for MH⁺ (UV/MS) 100/100.

### Example 75

### 8-Chloro-3-methoxy-11-(piperazin-1-yl)-dibenzo[b,f][1,4]oxazepine (189JO50B)

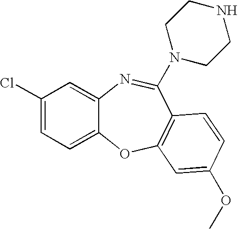

5-Chloro-2-fluoronitrobenzene (175 mg, 1 mmol) and methyl 2-hydroxy-4-methoxybenzoate (364 mg, 2 mmol) were reacted according to GP5 to give 6.8 mg of the title compound (189JO50B). MS (ESI) 344 (MH⁺). Purity for MH⁺ (UV/MS) 94/86.

### Example 76

### 8-Bromo-3-methoxy-11-(piperazin-1-yl)-dibenzo[b,f][1,4]oxazepine (189JO50D)

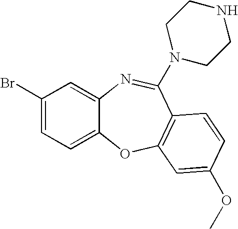

5-Bromo-2-fluoronitrobenzene (220 mg, 1 mmol) and methyl 2-hydroxy-4-methoxybenzoate (364 mg, 2 mmol) were reacted according to GP5 to give 14 mg of the title compound (189JO50D). MS (ESI) 388 (MH⁺). Purity for MH⁺ (UV/MS) 100/100.

### Example 77

### 3-Methoxy-11-(piperazin-1-yl)-dibenzo[b,f][1,4]oxazepine (189JO50E)

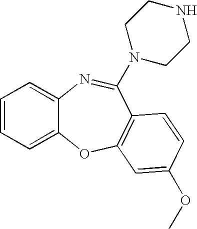

2-Fluoronitrobenzene (141 mg, 1 mmol) and methyl 2-hydroxy-4-methoxybenzoate (364 mg, 2 mmol) were reacted according to GP5 to give 33 mg of the title compound (189JO50E). MS (ESI) 310 (MH⁺). Purity for MH⁺ (UV/MS) 100/100.

### Example 78

### 7-Chloro-3-methoxy-11-(piperazin-1-yl)-dibenzo[b,f][1,4]oxazepine (189JO50F)

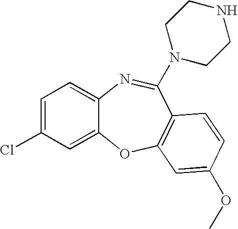

4-Chloro-2-fluoronitrobenzene (175 mg, 1 mmol) and methyl 2-hydroxy-4-methoxybenzoate (364 mg, 2 mmol) were reacted according to GP5 to give 6.7 mg of the title compound (189JO50F). MS (ESI) 344 (MH⁺). Purity for MH⁺ (UV/MS) 98/96.

### Example 79

### 8-Chloro-4-methyl-11-(piperazin-1-yl)-dibenzo[b,f][1,4]oxazepine (189JO50H)

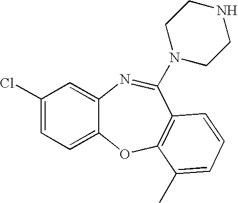

5-Chloro-2-fluoronitrobenzene (175 mg, 1 mmol) and methyl 2-hydroxy-3-methylbenzoate (332 mg, 2 mmol) were reacted according to GP5 to give 34 mg of the title compound (189JO50H). MS (ESI) 328 (MH⁺). Purity for MH⁺ (UV/MS) 100/100.

### Example 80

### 8-Bromo-4-methyl-11-(piperazin-1-yl)-dibenzo[b,f][1,4]oxazepine (189JO51A)

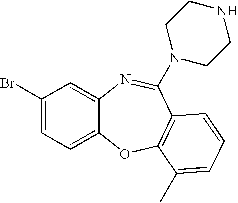

5-Bromo-2-fluoronitrobenzene (220 mg, 1 mmol) and methyl 2-hydroxy-3-methylbenzoate (332 mg, 2 mmol) were reacted according to GP5 to give 20 mg of the title compound (189JO51A). MS (ESI) 372 (MH⁺). Purity for MH⁺ (UV/MS) 100/100.

### Example 81

### 4-Methyl-11-(piperazin-1-yl)-dibenzo[b,f][1,4]oxazepine (189JO51B)

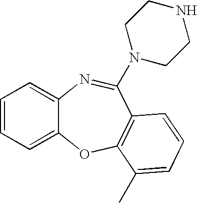

2-Fluoronitrobenzene (141 mg, 1 mmol) and methyl 2-hydroxy-3-methylbenzoate (332 mg, 2 mmol) were reacted according to GP5 to give 1.8 mg of the title compound (189JO51B). MS (ESI) 294 (MH⁺). Purity for MH⁺ (UV/MS) 99/98.

### Example 82

### 2-Bromo-8-chloro-11-(piperazin-1-yl)-dibenzo[b,f][1,4]oxazepine (189JO51D)

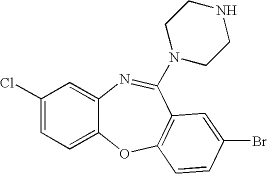

5-Chloro-2-fluoronitrobenzene (175 mg, 1 mmol) and methyl 5-bromo-2-hydroxybenzoate (462 mg, 2 mmol) were reacted according to GP5 to give 21 mg of the title compound (189JO51D). MS (ESI) 392 (MH⁺). Purity for MH⁺ (UV/MS) 100/100.

### Example 83

### 2,8-Dibromo-11-(piperazin-1-yl)-dibenzo[b,f][1,4]oxazepine (189JO51E)

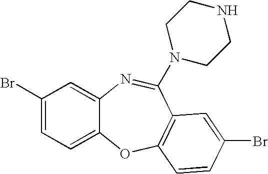

5-Bromo-2-fluoronitrobenzene (220 mg, 1 mmol) and methyl 5-bromo-2-hydroxybenzoate (462 mg, 2 mmol) were reacted according to GP5 to give 0.7 mg of the title compound (189JO51E). MS (ESI) 436 (MH⁺). Purity for MH⁺ (UV/MS) 94/99.

### Example 84

### 2-Bromo-1-(piperazin-1-yl)-dibenzo[b,f][1,4]oxazepine (189JO51F)

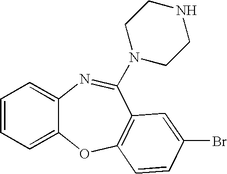

2-Fluoronitrobenzene (142 mg, 1 mmol) and methyl 5-bromo-2-hydroxybenzoate (462 mg, 2 mmol) were reacted according to GP5 to give 10 mg of the title compound (189JO51F). MS (ESI) 358 (MH⁺). Purity for MH⁺ (UV/MS) 95/99.

### Example 85

### 2-Bromo-7-chloro-11-(piperazin-1-yl)-dibenzo[b,f][1,4]oxazepine (189JO51G)

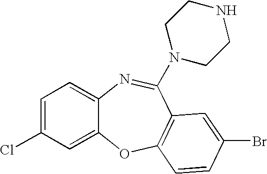

4-Chloro-2-fluoronitrobenzene (175 mg, 1 mmol) and methyl 5-bromo-2-hydroxybenzoate (462 mg, 2 mmol) were reacted according to GP5 to give 17 mg of the title compound (189JO51G). MS (ESI) 392 (MH⁺). Purity for MH⁺ (UV/MS) 100/100.

### Example 86

### 11-(piperazin-1-yl)-8-trifluoromethyl-dibenzo[b,f][1,4]oxazepine (189JO54A)

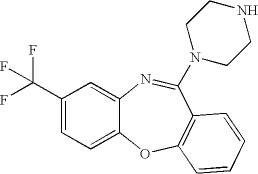

2-Fluoro-3-nitrobenzotrifluoride (209 mg, 1 mmol) and methyl 2-hydroxybenzoate (304 mg, 2 mmol) were reacted according to GP5 to give 19 mg of the title compound (189JO54A). MS (ESI) 348 (MH⁺). Purity for MH⁺ (UV/MS) 100/100.

### Example 87

### 4-Methyl-1-(piperazin-1-yl)-8-trifluoromethyl-dibenzo[b,f][1,4]oxazepine (189JO54C)

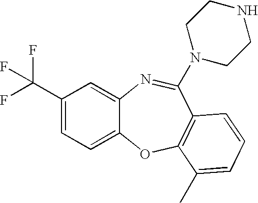

2-Fluoro-3-nitrobenzotrifluoride (209 mg, 1 mmol) and methyl 2-hydroxy-3-methylbenzoate (332 mg, 2 mmol) were reacted according to GP5 to give 15 mg of the title compound (189JO54C). MS (ESI) 362 (MH⁺). Purity for MH⁺ (UV/MS) 100/100.

### Example 88

### 8-Fluoro-11-(piperazin-1-yl)-dibenzo[b,f][1,4]oxazepine (189JO54E)

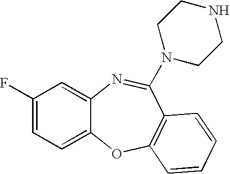

2,5-Difluoronitrobenzene (159 mg, 1 mmol) and methyl 2-hydroxybenzoate (304 mg, 2 mmol) were reacted according to GP5 to give 14 mg of the title compound (189JO54E). MS (ESI) 298 (MH⁺). Purity for MH⁺ (UV/MS) 100/100.

### Example 89

### 8-Fluoro-3-methoxy-11-(piperazin-1-yl)-dibenzo[b,f][1,4]oxazepine (189JO54F)

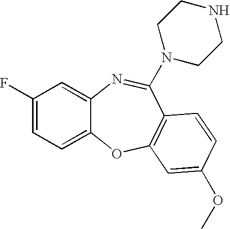

2,5-Difluoronitrobenzene (159 mg, 1 mmol) and methyl 2-hydroxy-4-methoxybenzoate (364 mg, 2 mmol) were reacted according to GP5 to give 9.8 mg of the title compound (189JO54F). MS (ESI) 328 (MH⁺). Purity for MH⁺ (UV/MS) 100/100.

### Example 90

### 8-Fluoro-4-methyl-11-(piperazin-1-yl)-dibenzo[b,f][1,4]oxazepine (189JO54G)

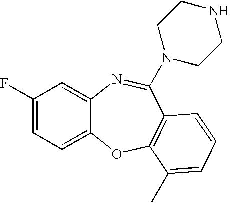

2,5-Difluoronitrobenzene (159 mg, 1 mmol) and 2-hydroxy-3-methylbenzoate (332 mg, 2 mmol) were reacted according to GP5 to give 9.8 mg of the title compound (189JO54G). MS (ESI) 312 (MH⁺). Purity for MH⁺ (UV/MS) 100/100.

### Example 91

### 2-Bromo-8-fluoro-11-(piperazin-1-yl)-dibenzo[b,f][1,4]oxazepine (189JO54H)

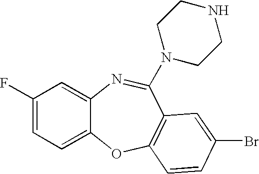

2,5-Difluoronitrobenzene (159 mg, 1 mmol) and methyl 5-bromo-2-hydroxybenzoate (462 mg, 2 mmol) were reacted according to GP5 to give 11 mg of the title compound (189JO54H). MS (ESI) 376 (MH⁺). Purity for MH⁺ (UV/MS) 100/100.

### Example 92

### 8-Methyl-1-(piperazin-1-yl)-dibenzo[b,f][1,4]oxazepine (189JO58A)

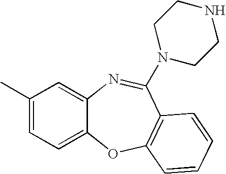

4-Fluoro-3-nitrotoluene (155 mg, 1 mmol) and methyl 2-hydroxybenzoate (304 mg, 2 mmol) were reacted according to GP5 to give 24 mg of the title compound (189JO58A). MS (ESI) 294 (MH⁺). Purity for MH⁺ (UV/MS) 100/98.

### Example 93

### 3-Methoxy-8-methyl-11-(piperazin-1-yl)-dibenzo[b,f][1,4]oxazepine (189JO58B)

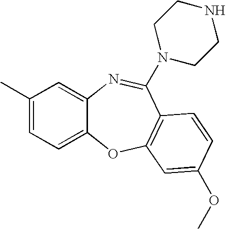

4-Fluoro-3-nitrotoluene (155 mg, 1 mmol) and methyl 2-hydroxy-4-methoxybenzoate (364 mg, 2 mmol) were reacted according to GP5 to give 27 mg of the title compound (189JO58B). MS (ESI) 324 (MH⁺). Purity for MH⁺ (UV/MS) 100/98.

### Example 94

### 4,8-Dimethyl-11-(piperazin-1-yl)-dibenzo[b,f][1,4]oxazepine (189JO58C)

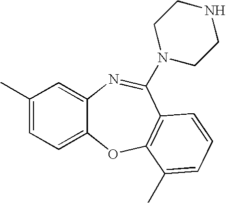

4-Fluoro-3-nitrotoluene (155 mg, 1 mmol) and methyl 2-hydroxy-3-methylbenzoate (332 mg, 2 mmol) were reacted according to GP5 to give 24 mg of the title compound (189JO58C). MS (ESI) 308 (MH⁺). Purity for MH⁺ (UV/MS) 100/98.

### Example 95

### 3-Methoxy-11-(piperazin-1-yl)-8-trifluoromethyl-dibenzo[b,f][1,4]oxazepine (189JO62A)

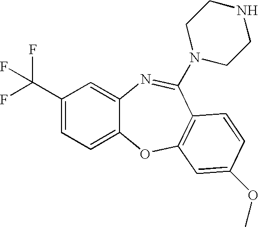

2-Fluoro-3-nitrobenzotrifluoride (209 mg, 1 mmol) and methyl 2-hydroxy-4-methoxybenzoate (364 mg, 2 mmol) were reacted according to GP5 to give 12 mg of the title compound (189JO62A). MS (ESI) 378 (MH⁺). Purity for MH⁺ (UV/MS) 100/95.

### Example 96

### 2-Bromo-11-(piperazin-1-yl)-8-trifluoromethyl-dibenzo[b,f][1,4]oxazepine (189JO62B)

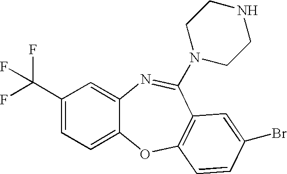

2-Fluoro-3-nitrobenzotrifluoride (209 mg, 1 mmol) and methyl 5-bromo-2-hydroxybenzoate (462 mg, 2 mmol) were reacted according to GP5 to give 11 mg of the title compound (189JO62B). MS (ESI) 426 (MH⁺). Purity for MH⁺ (UV/MS) 100/100.

### Example 97

### 6-Chloro-1-(piperazin-1-yl)-dibenzo[b,f][1,4]oxazepine (189JO69)

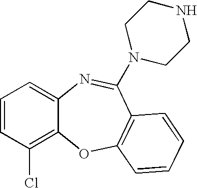

3-Chloro-2-fluoronitrobenzene (352 mg, 2 mmol) and methyl 2-hydroxybenzoate (453 mg, 3 mmol) were reacted according to GP5 to give 57 mg of the title compound (189JO69). MS (ESI) 314 (MH⁺). Purity for MH⁺ (UV/MS) 100/100.

### Example 98

### General Procedure 6 (GP6)

A mixture of a methyl aminobenzoic ester (1.0 mmol), a 2-fluoronitrobenezene (0.5 mmol) and Cs₂CO₃ (0.33 g, 1.0 mol) in DMF (3 mL) was stirred at 40° C. for 2 h. The mixture was diluted with EtOAc (10 mL) and washed with 2 M aqueous NaOH-solution (2×5 mL), dried (Na₂SO₄), concentrated, flash chromatographed (SiO₂, toluene:heptane:EtOAc-system), and concentrated. The residue was taken up in THF (4 mL), 1 M aqueous LiOH (3 mL) was added and the resulting mixture was stirred at 80° C. for 1 h, and then allowed to obtain room temperature. 2 M aqueous HCl was added until a pH of 2 was reached. The aqueous phase was extracted with EtOAc (3×). The combined organic phases were dried (Na₂SO₄) and concentrated. The residue was taken up in EtOH and a mixture of K₂CO₃ (0.35 g, 2.55 mmol) and Na₂S₂O₄ (0.44 g, 2.5 mmol) in water was added and the resulting mixture was stirred for 1 h. The mixture was diluted with water and washed with 1 M aqueous NaOH-solution (2×5 mL) and then dried (Na₂SO₄) and concentrated.

The residue was taken up in CH₃CN, 1-ethyl-3-(3-dimethylaminopropyl)carbodiimide hydrochloride (143 mg 0.75 mmol), 1-hydroxybenzotriazole hydrate (160 mg, 0.75 mmol), triethylamine (311 μL, 2.25 mmol), and N,N-dimethylaminopyridine (catalytic amount) were added. The resulting mixture was heated in a capped tube using microwave assisted heating (140° C., 10 min). The mixture was diluted with EtOAc, washed with saturated aqueous NaHCO₃-solution, dried (Na₂SO₄) and concentrated. The residue was taken up in dioxane and added to a mixture of TiCl₄ (0.55 mL, 0.55 mmol, 1 M in toluene) and piperazine (0.22 g, 2.5 mmol) in dioxane at 50° C. The resulting mixture was stirred at 100° C. over night, and then allowed to obtain room temperature. To the mixture was added aqueous HCl (3 mL, 2 M) and then the aqueous phase was extracted with EtOAc (2×4 mL). To the aqueous phase was added aqueous NaOH (6 mL, 2 M) and the resulting suspension was extracted with EtOAc (3×3 mL). The combined organic phases were concentrated and purified by HPLC.

### Example 99

### 2-Bromo-8-methyl-11-(piperazin-1-yl)-dibenzo[b,f][1,4]oxazepine (189JO63A)

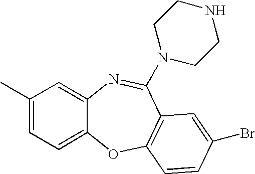

4-Fluoro-3-nitrotoluene (78 mg, 0.5 mmol) and methyl 5-bromo-2-hydroxybenzoate (231 mg, 1 mmol) were reacted according to GP6 to give 13 mg of the title compound (189JO63A). MS (ESI) 372 (MH⁺). Purity for MH⁺ (UV/MS) 100/100.

### Example 100

### 7-Chloro-4-methyl-11-(piperazin-1-yl)-dibenzo[b,f][1,4]oxazepine (189JO63B)

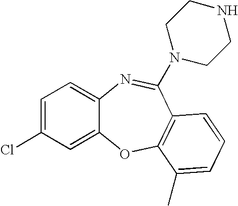

4-Chloro-2-fluoronitrobenzene (88 mg, 0.5 mmol) and methyl 2-hydroxy-3-methylbenzoate (166 mg, 1 mmol) were reacted according to GP6 to give 24 mg of the title compound (189JO63B). MS (ESI) 328 (MH⁺). Purity for MH⁺ (UV/MS) 100/100.

### Example 101

### 8-Phenyl-11-(piperazin-1-yl)-dibenzo[b,f][1,4]oxazepine (189JO64)

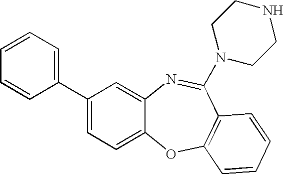

To a mixture of 8-bromo-10H-dibenzo[b,f][1,4]oxazepin-11-one (189JO56) (30 mg, 0.12 mmol), benzene boronic acid (18 mg, 0.15 mmol) and K₂CO₃ (34 mg, 0.24 mmol) in deoxygenised toluene/EtOH/H₂O (1.5 mL) was added tetrakis(triphenylphosphine)palladium(0) (catalytic amount) and the resulting mixture was heated in a capped tube in a microwave oven (140° C., 15 min). The mixture was diluted with EtOAc, washed with saturated aqueous NaHCO₃-solution, dried (Na₂SO₄) and concentrated to give crude 8-phenyl lactam. A mixture of the intermediate 8-phenyl lactam in dioxane (1 mL) was added to a mixture of TiCl₄ (0.27 mL, 0.27 mmol, 1 M in toluene) and piperazine (0.103 g, 1.2 mmol) in dioxane at 50° C. The resulting mixture was stirred at 100° C. over night and then allowed to obtain room temperature. To the mixture was added aqueous HCl (3 mL, 2 M) and then the aqueous phase was extracted with EtOAc (2×4 mL). To the aqueous phase was added aqueous NaOH (6 mL, 2 M) and the resulting suspension was extracted with EtOAc (3×3 mL). The combined organic phases were applied onto a SCX-2 ion exchange column. The column was washed with MeOH, and then the product was eluted with NH₃ (7 N in MeOH) concentrated and purified by HPLC to give 16 mg of the title compound (189JO64). MS (ESI) 356 (MH⁺). Purity for MH⁺ (UV/MS) 100/99.

### Example 102

### 8-Chloro-11-(piperidin-4-yl)-5H-dibenzo[b,e][1,4]diazepine (160FE67A)

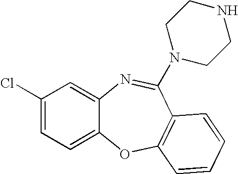

4-CBZ-piperidylzinc iodide (generated from of 4-CBZ-piperidyl iodide (345 mg, 1.0 mmol) using zinc metal and dibromoethane) (0.8 mmol) was added at 50° C. to a solution of 8,5-dichloro-5H-dibenzo[b,e][1,4]diazepine (160FE64) (106 mg, 0.4 mmol) and PdCl₂(PPh₃)₂ (18 mg, 0.04 mmol) in dry THF (2 ml). The reaction was shaken for 16 h and then quenched by the addition of aqueous saturated NH₄Cl-solution. The resulting mixture was extracted twice with ether and the combined ethereal phases were washed with brine and dried (Na₂SO₄). Filtration followed by concentration at reduced pressure of the organic phase gave a crude product. BBr₃ (100 μl) added at −30° C. was added to the crude product dissolved in CH₂Cl₂ (1 ml). The reaction temperature was then slowly increased to 0° C. TLC indicated complete conversion of the starting material and Et₃N, H₂O and EtOAc were sequentially added to the reaction mixture. The organic phase was washed with brine and dried (Na₂SO₄). Filtration followed by concentration at reduced pressure gave a crude product, which was purified by HPLC to give 2.3 mg of the title compound (160FE67A). MS (ESI) 312 (MH⁺). Purity for MH⁺ (UV/MS) 99/96.

### Example 103

### 5-Benzyl-8-chloro-11-(piperidin-4-yl)-5H-dibenzo[b,e][1,4]diazepine (160FE67B)

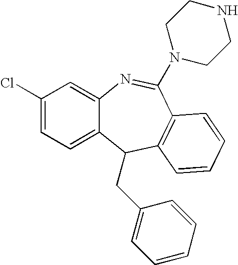

4.4 mg of the title compound (160FE67B) was isolated as a by-product in the synthesis of Example 102. MS (ESI) 402 (MH⁺). Purity for MH⁺ (UV/MS) 85/87.

### Example 104

### General Procedure 7 (GP7)

A mixture of a 2-aminobenzoic acid (1 eq.), a 2-fluoronitrobenezene (2 eq. or 3 eq.) and K₂CO₃ (3 eq.) in DMF was heated to 100° for 2 hour then allowed to obtain room temperature. The organic phase was extracted with 0.1 M aqueous NaOH-solution (3 x). The combined aqueous phases were acidified with 4 M aqueous HCl and extracted with EtOAc (3×). The combined organic phases were dried (Na₂SO₄) and concentrated. The residue was taken up in EtOH and a solution of K₂CO₃ (5 eq.) and Na₂S₂O₄ (5 eq.) in water was added and the resulting mixture was stirred for 1 h. The mixture was concentrated and the residue taken up in EtOAc. The mixture was acidified with aqueous HCl (2 M) and then the aqueous phase was extracted with EtOAc (3×) and the combined organic phases were concentrated.

The residue was taken up in xylene and the resulting mixture was stirred at 130° C. over night. The mixture was diluted with EtOAc, washed with saturated aqueous NaHCO₃-solution, dried (Na₂SO₄), concentrated, and flash chromatographed (SiO₂, heptane:EtOAc system)

### Example 105

### 8-Bromo-5,10-dihydro-dibenzo[b,e][1,4]diazepine-11-one (166JO31)

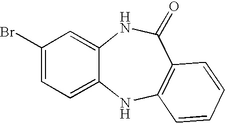

5-Bromo-2-fluoronitrobenzene (1.6 g, 7.4 mmol) and 2-aminobenzoic acid (0.50 g, 3.6 mmol) were reacted according to GP7 to give 331 mg of the title compound (166JO31). MS (ESI) 289 (MH⁺). Purity for MH⁺ (UV) 93%.

### Example 106

### 5,10-Dihydro-dibenzo[b,e][1,4]diazepine-11-one (160FE15A)

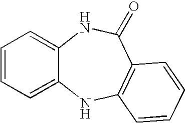

2-Fluoronitrobenzene (847 g, 6 mmol) and 2-aminobenzoic acid (274 mg, 2.0 mmol) were reacted according to GP7 to give 130 mg of the title compound (160FE15A).

### Example 107

### 8-Fluoro-5,10-dihydro-dibenzo[b,e][1,4]diazepine-11-one (160FE15C)

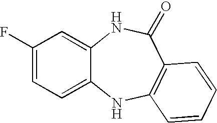

2,4-Difluoronitrobenzene (0.96 g, 6 mmol) and 2-aminobenzoic acid (274 mg, 2.0 mmol) were reacted according to GP7 to give 100 mg of the title compound (160FE15C).

### Example 108

### 8,5-Dichloro-5H-dibenzo[b,e][1,4]diazepine (160FE64)

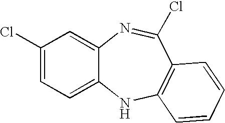

N,N-dimethylaniline (5.1 ml, 40 mmol) and phosphorus oxychloride (2.8 ml, 30 mmol) was added to a mixture of 8-chloro-5,10-dihydro-dibenzo[b,e][1,4]diazepine-11-one (2.45 g, 10 mmol) in dry toluene (20 ml). The mixture was shaken at 95° C. for 2 h. The temperature was then decreased and the excess N,N-dimethylaniline and phosphorus oxychloride were removed at reduced pressure using an oil pump. The remaining oil was dissolved in dioxane (20 ml) and aqueous Na₂CO₃-solution (10 ml, 2 M) was added. The two-phase mixture was shaken at 80° C. for 30 min. The temperature was then decreased and ether was added to the reaction mixture. The ethereal phase was washed with saturated aqueous NaCl-solution, dried (Na₂SO₄) and finally concentrated at reduced pressure. The obtained oil crystallized upon standing at room temperature. Recrystallization (heptane-ether) gave 1.8 g (69%) of the title compound (160FE64). ¹H NMR (CDCl₃) δ 7.61 (dd, 1H, J=1.4, 7.8 Hz), 7.31 (dt, 1H, J=1.5, 8.0 Hz), 7.15 (d, 1H, J=2.5 Hz), 7.02 (m 2H), 6.66 (dd, 1H, J=1.0, 7.8 Hz), 6.58 (d, 1H, J=8.4 Hz), 4.94 (bs, 1H). ¹³C NMR (CDCl₃) δ 157.2, 152.4, 140.3, 138.9, 134.0, 131.9, 129.7, 128.5, 128.0, 127.0, 123.5, 121.0, 119.8.

### Example 109

### 8-Chloro-11-methylsulfanyl-5H-dibenzo[b,e][1,4]diazepine (166JO50)

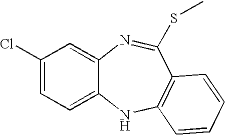

A mixture of 8-chloro-5,10-dihydro-dibenzo[b,e][1,4]diazepine-11-one (500 mg, 2.05 mmol) and 2,4-bis(4-methoxyphenyl)-1,3-dithia-2,4-diphosphetane-2,4-disulfide (480 mg, 1.19 mmol) in toluene (4 mL) was heated in a capped tube in a microwave oven (120° C., 30 minutes). The mixture was chromatographed (SiO₂, heptane:EtOAc, 2:1) to give 599 mg of the intermediate thiolactam. To a mixture of the intermediate thiolactam in THF (10 mL) was added MeI (633 μL, 10.3 mmol) and the resulting mixture was heated at reflux for 4 h. The mixture was concentrated to give 610 mg of the crude title compound (166JO50) (purity 50%).

### Example 110

### N,N-diethyl(2-bromobenzyl)amide (189JO10)

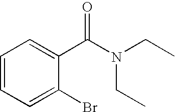

To a mixture of 2-bromo benzoylchloride (3.5 g, 16 mmol) in CH₂Cl₂ (50 mL) at 0° C. was added diethylamine (3.2 mL, 32 mmol) drop-wise and the resulting mixture was allowed to obtain room temperature. After 30 minutes, water was added, the mixture was diluted with CH₂Cl₂, washed with saturated aqueous NaHCO₃-solution and saturated aqueous NH₄Cl-solution, dried (Na₂SO₄) and concentrated to give 3.9 g (95%) of the title compound (189JO10). ¹H NMR (CDCl₃) δ 7.54 (m, 1H), 7.32 (m, 1H), 7.22 (m, 2H), 3.79 (m, 1H), 3.33 (m, 1H), 3.13 (m, 2H), 1.26 (t, 3H, J=7.2 Hz), 1.05 (t, 3H, J=7.0 Hz). ¹³C NMR (CDCl₃) δ 168.5, 139.0, 132.8, 130.0, 127.61, 127.59, 119.3, 42.8, 39.0, 14.0, 12.6

### Example 111

### 2[(4-Chloro-2-methylphenyl)(4-methoxybenzyl)amino]-N,N-diethylbenzamide (189JO26)

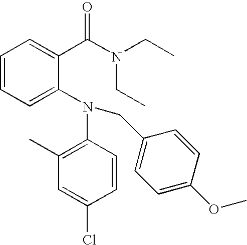

To a mixture of N,N-diethyl(2-bromobenzyl)amide (189JO10) (1.41 g, 5.50 mmol) and 4-chloro-2-methylaniline (1.01 g, 7.15 mmol) in deoxygenised toluene (14 mL) was added NaO^{t}Bu (0.74 g, 7.7 mmol), rac-BINAP (110 mg, 0.17 mmol) and Pd(OAc)₂ (18 mg, 0.08 mmol) and the resulting mixture was stirred under Ar for 14 h at 80° C. The mixture was filtered through celite, concentrated and flash chromatographed (SiO₂, heptane:EtOAc, 10:1-4:1) which gave unprotected intermediate ketone (1.50 g) containing about 15% impurities.

The mixture containing the intermediate was dissolved in DMF (20 mL). p-Methoxybenzyl chloride (0.90 mL, 6.6 mmol) was added and then NaH (0.23 g, 5.6 mmol, 60% in mineral oil) was added portions-wise. The resulting mixture was stirred at room temperature for 1 h, and then quenched by addition of saturated aqueous NaHCO₃-solution. The mixture was diluted with EtOAc, washed with saturated aqueous NaHCO₃-solution, dried (Na₂SO₄), concentrated and flash chromatographed (SiO₂, toluene: EtOAc 10:1) to give 1.66 g (68%) of the title compound (189JO26). ¹H NMR (CDCl₃) δ 7.35 (m, 2H), 7.20 (m, 1H), 7.09-6.99 (m, 4H), 6.91 (m, 2H), 6.80 (m, 2H), 4.84/4.54 (Abq, 2H, J=16.2 Hz), 3.74 (s, 3H), 3.18 (m, 2H), 3.03 (m, 1H), 2,48 (m, 1H), 2.17 (s, 3H), 1.01 (t, 3H, J=7.2 Hz), 0.97 (t, 3H, J=7.0 Hz), ¹³C NMR (CDCl₃) δ 169.6, 158.7, 146.53, 146.51, 137.0, 131.3, 130.9, 130.4, 129.6, 129.3, 128.7, 127.8, 127.4, 126.3, 122.8, 121.4, 114.0, 57.1, 55.3, 43.3, 39.0, 19.1, 13.9, 12.9. MS (ESI) 437 (MH⁺).

### Example 112

### 2-Chloro-5-(4-methoxybenzyl)-5,11-dihydrodibenzo[b,f]azepin-11-one (189JO27)

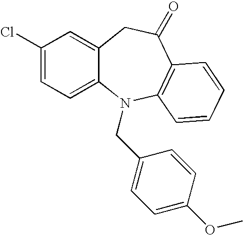

To a mixture of diisopropylamine (1.09 mL, 7.8 mmol) and N,N,N,N-tetramethylenediamine (1.17 mL, 7.8 mmol) in dry THF (19 mL) at −20° C. was added n-BuLi (5.54 mL, 1.4 M in hexane) and the resulting mixture was stirred at −20° C. for 5 minutes. Then a mixture of 2[(4-chloro-2-methylphenyl)(4-methoxybenzyl)-amino]-N,N-diethylbenzamide (189JO26) (1.36 g, 3.1 mmol) in dry THF (38 mL) was added and the resulting mixture was stirred at −20° for 4 h. The reaction was quenched by addition of saturated aqueous NH₄Cl-solution. The mixture was diluted with EtOAc, washed with water, dried (Na₂SO₄), concentrated, and flash chromatographed (SiO₂, toluene:heptane, 7:1-1:0) to give 665 mg (59%) of the title compound (189JO27). ¹H NMR (CDCl₃) δ 8.15 (dd, 1H, J=1.8, 8.0 Hz), 7.43 (m, 1H), 7.24 (m, 4H), 7.17 (d, 1H, J=8.6 Hz), 7.12 (dd, 1H, J=2.4, 8.6 Hz), 7.00 (dt, 1H, J=0.8, 7.0 Hz), 6.81 (m, 2H), 5.09 (s, 2H), 4.00 (s, 2H), 3.75 (s, 3H); ¹³C NMR (CDCl₃) δ 190.3, 159.1, 149.5, 146.2, 134.1, 132.4, 131.3, 131.1, 129.1, 129.0, 128.6, 127.3, 126.4, 123.4, 121.0, 118.5, 114.2, 55.5, 19.3. MS (ESI) 364 (MH⁺).

### Example 113

### 2-(4-Chloro-2-nitro-phenylsulfanyl)-benzoic acid methyl ester (189JO09)

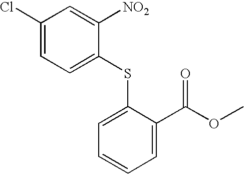

To a mixture of 5-chloro-2-nitrofluorobenzene (176 mg, 1 mmol) and methyl thiosalicylate (275 μL, 2 mmol) in DMF (5 mL) was added Cs₂CO₃ (652 mg, 2 mmol) and the resulting mixture was stirred at room temperature for 2 h. The mixture was diluted with CH₂Cl₂, washed with water, dried (Na₂SO₄), concentrated and flash chromatographed (SiO₂, heptane:toluene, 1:10-1:4) to give 300 mg (92%) of the title compound (189JO09). ¹H NMR (CDCl₃) δ 8.15 (d, 1H J=2.4 Hz), 7.94 (m, 1H), 7.53-7.46 (m, 3H), 7.34 (dd, 1H, 2.4, 8.6 Hz), 6.95 (d, 1H, J=8.8 Hz), 3.82 (s, 3H).

### Example 114

### 2-(2-Amino-4-chlorophenylsulfanyl)-benzoic acid methyl ester (189JO11)

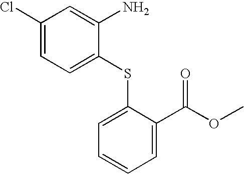

To a mixture of 2-(4-chloro-2-nitro-phenylsulfanyl)-benzoic acid methyl ester (189JO09) (232 mg, 0.72 mmol) in EtOH (5 mL) was added SnCl₂.2H₂O (812 mg, 3.6 mmol) and the resulting mixture was stirred at 80° C. for 2 h and then concentrated. The residue was treated with ice, and then Na₂CO₃ was added until a pH of 10 was reached. EtOAc was added and the slurry was filtered through celite. The EtOAc-phase was washed with water and brine, dried (Na₂SO₄) and concentrated to give 149 mg (70%) of the title compound (189JO31). ¹H NMR (CDCl₃) δ 8.02 (dd, 1H, J=1.6, 7.8 Hz), 7.39 (d, 1H, J=8.2 Hz), 7.29 (m, 1H), 7.15 (dt, 1H, J=1.2, 7.8 Hz), 6.87 (d, 1H, J=2.2 Hz), 6.80 (dd, 1H, J=2.2, 8.2 Hz), 6.76 (dd, 1H, J=1.2, 8.0 Hz), 3.96 (s, 3H).

### Example 115

### 8-Chloro-10H-dibenzo[b,f][1,4]thiazepin-11-one (189JO13)

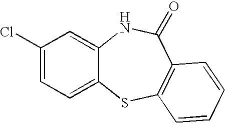

A mixture of 2-(2-amino-4-chlorophenylsulfanyl)-benzoic acid methyl ester (189JO11) (149 mg, 0.51 mmol) and AlMe₃ (355 μL, 0.71 mmol, 2 M in toluene) in CH₂Cl₂ (3 mL) was stirred at ambient temperature for six days, and then water was added carefully. The mixture was diluted with CH₂Cl₂, and was acidified with 2 M aqueous HCl. The organic phase was separated, dried (Na₂SO₄), concentrated and flash chromatographed (heptane:EtOAc, 5:1-3:1) to give 38 mg (29%) of the title compound (189JO13). MS (ESI) 262 (MH⁺).

### Example 116

### 2-(Chloro-2-nitro-phenoxy)-benzoic acid methyl ester (189JO29A)

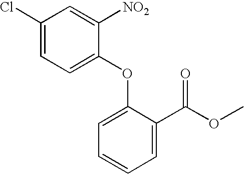

Cs₂CO₃ (1.30 g, 4 mmol) was added to a mixture of 5-chloro-2-nitrofluorobenzene (352 mg, 2 mmol) and methyl 2-hydroxybenzoate (0.52 mL, 4 mmol) in DMF (6 mL) and the resulting mixture was stirred at room temperature for 2 h. The mixture was diluted with CH₂Cl₂, washed with water, dried (Na₂SO₄), concentrated and flash chromatographed (SiO₂, heptane:EtOAc, 10:1-4:1) to give 505 mg (82%) of the title compound (189JO29A). ¹H NMR (CDCl₃) δ 8.02 (dd, 1H, J=1.8, 7.8 Hz), 7.96 (d, 1H, J=1.9 Hz), 7.59 (dt, 1H, J=2.0, 7.6 Hz), 7.39 (dd, 1H, J=2.5, 9.0 Hz), 7.24 (dt, 1H, J=1.2, 7.6 Hz), 7.13 (dd, 1H, J=1.2, 8.0 Hz), 6.74 (d, 1H, J=9.0 Hz), 3.77 (s, 3H).

### Example 117

### 8-Chloro-10H-dibenzo[b,f][1,4]oxazepin-11-one (189JO29C

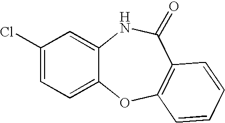

Pd (catalytic amount, 5% on carbon) was added to a solution of 2-(chloro-2-nitro-phenoxy)-benzoic acid methyl ester (189JO29A) (505 mg, 1.64 mmol) in EtOAc (20 mL) and the resulting mixture was hydrogenated (H₂, 1 atm.) for 48 h, then filtered through celite and concentrated. The residue was taken up in toluene (6 mL) and NaH (160 mg, 4.0 mmol, 60% in mineral oil) was added. The resulting mixture was stirred at 80° C. over night, and then quenched by addition of saturated aqueous NH₄Cl-solution. The resulting mixture was diluted with EtOAc, washed with water, dried (Na₂SO₄), concentrated and flash chromatographed (SiO₂, toluene:EtOAc, 4:1), which gave 171 mg (42%) of the title compound (189JO29C). ¹H NMR (CDCl₃) δ 8.12 (bs, 1H), 7.95 (dd, 1H, J=1.8, 8.0 Hz), 7.54 (dt, 1H, J=1.8, 8.0 Hz), 7.29-7.19 (m, 3H), 7.08 (dd, 1H, J=2.3, 8.6 Hz), 7.04 (d, 1 H, J=2.3 Hz). MS (ESI) 246 (MH⁺).

### Example 118

### 3-Chloro-5,11-dihydro-dibenzo[b,e]azepin-6-one (189JO59)

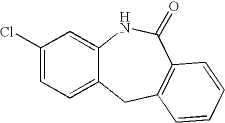

To a mixture of 5-chloro-2-methylphenyl isocyanate (100 μL, 0.73 mmol) in CCl₄ (2 mL) was added sulfuryl chloride (118 μL, 0.88 mmol) and 2,2′-azobis(isobutyronitrile) (catalytic amount) and the resulting mixture was refluxed for 20 h. The mixture was allowed to obtain room temperature, then diluted with CH₂Cl₂, washed with saturated aqueous NaHCO₃-solution, dried (Na₂SO₄) and concentrated. The mixture was taken up in benzene (2 mL) and a mixture of AlCl₃ (160 mg, 1.2 mmol) in benzene (1 mL) was added. The resulting mixture was stirred at 80° for 4 h, and then allowed to obtain room temperature. The mixture was filtered through a short column (SiO₂, heptane:EtOAc, 1:1) to give 25 mg (14%) of the title compound (189JO59). ¹H NMR (CDCl₃) δ 8.18 (bs, 1H), 7.92 (dd, 1H, J=1.2, 7.8 Hz), 7.46 (dt 1H, J=1.4, 7.4 Hz), 7.34 (dt, 1H, J=1.2, 7.4 Hz), 7.23 (m, 2H), 7.07 (m, 2H), 3.92 (s 2H). MS (ESI) 244 (MH⁺)

### Example 119

### 8-Bromo-10H-dibenzo[b,f][1,4]oxazepin-11-one (189JO56)

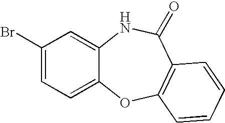

A mixture of a methyl 2-hydroxybenzoate (1.0 mL, 10.0 mmol), 5-bromo-2-fluoronitrobenezene (0.62 mL, 5.0 mmol) and Cs₂CO₃ (3.3 g, 10.0 mol) in DMF (12 mL) was stirred at 40° C. for 2 h. The mixture was diluted with EtOAc and washed with 2 M aqueous NaOH-solution. To the EtOAc-phase was added EtOH, H₂O, K₂CO₃ (2.8 g, 20 mmol) and Na₂S₂O₄ (3.5 g, 20 mmol) and the resulting mixture was stirred vigorously for 1 h. The aqueous phase was removed and the organic phase was washed with 1 M aqueous NaOH-solution and then concentrated. The residue was taken up in DMF (1 mL) and then toluene (4 mL) and NaH (60 mg, 1.5 mmol, 60% in mineral oil) were added and the resulting mixture was stirred at 80° C. over night, then quenched by addition of saturated aqueous NH₄Cl-solution. The resulting mixture was diluted with EtOAc, washed with 2 M aqueous NaOH-solution, dried (Na₂SO₄), concentrated, filtered through a short SiO₂-column, concentrated and crystallised from heptane:EtoAc to give 130 mg of the title compound (189JO56). MS (ESI) 290 (MH⁺). Purity for MH⁺ (UV/MS) 100/100.

### Example 120

### General Procedure 8 (GP8)

A BOC-protected diamine (1.8 eq.) was added to 8-chloro-11-methylsulfanyl-5H-dibenzo[b,e][1,4]diazepine (166JO50) (purity 50%, 1 eq.) in pyridine. The resulting mixture was heated in a capped tube at 110° C. for 66 h. The mixture was concentrated and then diluted with CH₂Cl₂:trifluoroacetic acid (2:1-ratio). The resulting mixture was stirred at ambient temperature over night, and then concentrated. The residue was taken up in CH₂Cl₂ and washed with saturated aqueous NaHCO₃-solution. The organic phase was applied onto a SCX-2 ion exchange column. The column was washed with MeOH, and then the product was eluted with NH₃ (7 N in MeOH), concentrated and purified on HPLC.

### Example 121

### (8-Chloro-5H-dibenzo[b,e][1,4]diazepin-11-yl)-(S)-1-pyrrolidin-2-yl-methyl-amine (166JO51)

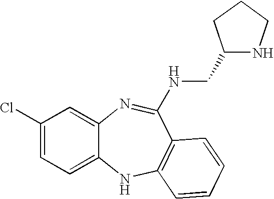

8-Chloro-11-methylsulfanyl-5H-dibenzo[b,e][1,4]diazepine (166JO50) (50 mg, 0.11 mmol) and (S)-(2-aminomethyl)-1-N-(tert-butoxycarbonylamino)pyrrolidine (39 mg, 0.2 mmol) were reacted according to GP8 to give 3.0 mg of the title compound (166JO51). MS (ESI) 327 (MH⁺). Purity for MH⁺ (UV/MS) 100/92.

### Example 122

### 1-(8-Chloro-5H-dibenzo[b,e][1,4]diazepin-11-yl)-piperidine-4-yl-amine (166JO55)

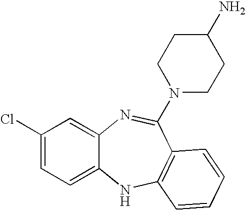

8-Chloro-11-methylsulfanyl-5H-dibenzo[b,e][1,4]diazepine (166JO50) (50 mg, 0.11 mmol) and 4-(tert-butoxycarbonylamino)aminopiperidine (39 mg, 0.2 mmol) were reacted according to GP8 to give 6.5 mg of the title compound (166JO55). MS (ESI) 327 (MH⁺). Purity for MH⁺ (UV/MS) 100/99.

### Example 123

### 1-(8-Chloro-5H-dibenzo[b,e][1,4]diazepin-11-yl)-pyrrolidin-3-yl-amine (166JO64)

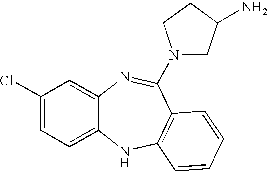

8-Chloro-11-methylsulfanyl-5H-dibenzo[b,e][1,4]diazepine (166JO50) (100 mg, 0.22 mmol) and 3-(tert-butoxycarbonylamino)pyrrolidine (73 mg, 0.4 mmol) were reacted according to GP8 to give 8.1 mg of the title compound (166JO64). MS (ESI) 313 (MH⁺). Purity for MH⁺ (UV/MS) 100/94.

### Example 124

### (8-Chloro-5H-dibenzo[b,e][1,4]diazepin-11-yl)-(R)-1-pyrrolidin-2-yl-methyl-amine (166JO70)

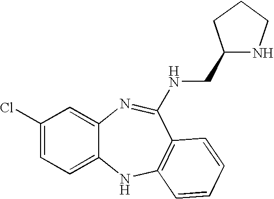

8-Chloro-11-methylsulfanyl-5H-dibenzo[b,e][1,4]diazepine (166JO50) (100 mg, 0.22 mmol) and (R)-(2-aminomethyl)-1-N-(tert-butoxycarbonylamino)pyrrolidine (78 mg, 0.4 mmol) were reacted according to GP8 to give 7.6 mg of the title compound (166JO70). MS (ESI) 327 (MH⁺). Purity for MH⁺ (UV/MS) 100/90.

### Example 125

### (8-Chloro-5H-dibenzo[b,e][1,4]diazepin-11-yl)-pyrrolidin-3-yl-amine (166JO74)

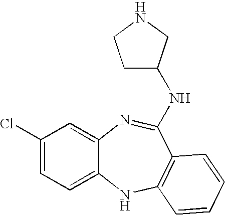

8-Chloro-11-methylsulfanyl-5H-dibenzo[b,e][1,4]diazepine (166JO50) (100 mg, 0.22 mmol) and 3-amino-1-N-(tert-butoxycarbonylamino)pyrrolidine (73 mg, 0.4 mmol) were reacted according to GP8 to give 7.7 mg of the title compound (166JO74). MS (ESI) 313 (MH⁺). Purity for MH⁺ (UV/MS) 100/90.

### Example 126

### 8-Chloro-11-(2,5-diaza-bicyclo[2.2.1]hept-2-yl)-5H-dibenzo[b,e][1,4]diazepine (166JO39-2)

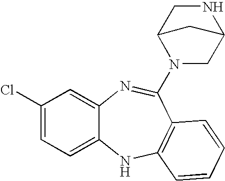

8-Chloro-11-methylsulfanyl-5H-dibenzo[b,e][1,4]diazepine (166JO50) (50 mg, 0.11 mmol) and N-(tert-butoxycarbonylamino)-2,5-diazabicyclo[2.2.1]heptane (34 mg, 0.2 mmol) were reacted according to GP8 to give 15 mg of the title compound (166JO39-2). MS (ESI) 324 (MH⁺). Purity for MH⁺ (UV/MS) 93/100.

### Example 127

### Acetidin-3-yl-(8-chloro-5H-dibenzo[b,e][1,4]diazepine-11-yl)amine (189JO65)

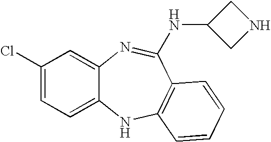

To 8,5-Dichloro-5H-dibenzo[b,e][1,4]diazepine (160FE64) (30 mg, 0.11 mmol) in dioxane (2.0 mL) was added 3-amino-azetidine-1-carboxylic acid tert-butyl ester (59 mg, 0.34 mmol) and Cs₂CO₃ (74 mg, 0.23 mmol) and the resulting mixture was heated in capped tube using microwave assisted heating (170° C., 40 minutes). The mixture was diluted with EtOAc, washed with water, dried (Na₂SO₄) and concentrated. The residue was taken up in CH₂Cl₂ (2 mL) and trifluoroacetic acid (1 mL) was added. The resulting mixture was stirred at ambient temperature over night, and then concentrated. The residue was taken up in CH₂Cl₂ and washed with saturated aqueous NaHCO₃-solution. The organic phase was applied onto a SCX-2 ion exchange column. The column was washed with MeOH, and then the product was eluted with NH₃ (7 N in MeOH), concentrated, and purified by HPLC to give 16 mg of the title compound (189JO65). MS (ESI) 299 (MH⁺). Purity for MH⁺ (UV/MS) 97/90.

### Example 128

### General Procedure 9 (GP9)

A mixture of a 3-aminomethyl ester (1 eq.), 5-bromo-2-fluoronitrobenezene (1 eq.) and K₂CO₃ (4 eq.) in DMF was heated to 60° C. for 1 hour, and then allowed to obtain room temperature. The mixture was diluted with CH₂Cl₂ and washed with saturated aqueous NH₄Cl-solution, dried (Na₂SO₄) and concentrated. The residue was taken up in EtOH and a mixture of K₂CO₃ (5 eq.) and Na₂S₂O₄ (5 eq.) in water was added and the resulting mixture was stirred vigorously for 1 h. The aqueous phase was extracted with EtOAc (3×) and the combined organic phases were dried (Na₂SO₄) and concentrated.

The residue was taken up in CH₃CN, H₂SO₄ (10 vol-%, 98%) was added, and the resulting mixture was stirred at 80° C. for 1 h. The mixture was diluted with CH₂Cl₂, washed with saturated aqueous NaHCO₃-solution, dried (Na₂SO₄), concentrated, flash chromatographed (SiO₂, heptane:EtOAc-system), and concentrated to give intermediate lactam.

The residue was taken up in dioxane and added to a mixture of TiCl₄ (1.1 eq., 1 M in toluene) and piperazine (5 eq.) in dioxane at 50° C. The resulting mixture was stirred at 100° C. over night, and then allowed to obtain room temperature. To the mixture was added aqueous HCl (2 M) until acidic solution and then the aqueous phase was extracted with EtOAc (2×). To the aqueous phase was added aqueous NaOH (2 M) until basic solution and the resulting suspension was extracted with EtOAc (3×). The combined organic phases were concentrated and flash chromatographed (SiO₂, CH₂Cl₂:MeOH, NH₃ (7N in MeOH))-system.

### Example 129

### 7-Bromo-4-(piperazin-1-yl)-2,3-dihydro-1H-benzo[b][1,4]diazepine (166JO47)

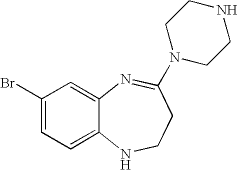

5-Bromo-2-fluoronitrobenzene (440 mg, 2.0 mmol) and methyl 3-amino propionate hydrochloride (920 mg, 3.0 mmol) were reacted according to GP9 to give 4.0 mg of the title compound (166JO47). MS (ESI) 309 (MH⁺). Purity for MH⁺ (UV/MS) 100/100.

### Example 130

### 7-Bromo-2-methyl-(piperazin-1-yl)-2,3-dihydro-1H-benzo[b][1,4]diazepine (166JO95)

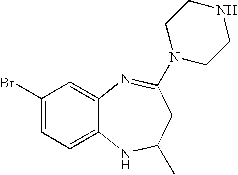

5-Bromo-2-fluoronitrobenzene (440 mg, 2.0 mmol) and methyl 3-amino buturate (787 mg, 3.0 mmol) were reacted according to GP9 to give 12 mg of the title compound (166JO95). MS (ESI) 323 (MH⁺). Purity for MH⁺ (UV/MS) 100/100.

### Example 131

### 7-Bromo-2-phenyl-4-(piperazine-1-yl)-2,3-dihydro-1H-benzo[b][1,4]diazepine (189JO20)

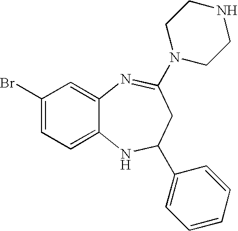

5-Bromo-2-fluoronitrobenzene (440 mg, 2.0 mmol) and ethyl 3-amino-3-phenylpropionate hydrochloride (394 mg, 1.5 mmol) were reacted according to GP9 to give 9.8 mg of the title compound (189JO20). MS (ESI) 385 (MH⁺). Purity for MH⁺ (UV/MS) 97/88.

### Example 132

### 7-Bromo-10-(piperazin-1-yl)-1,2,3,3a,4,10a-hexahydro-benzo[b]cyclopenta[e][1,4]diazepine (166JO46)

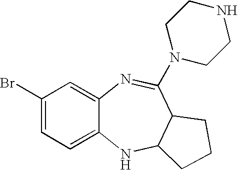

5-Bromo-2-fluoronitrobenzene (110 mg, 0.5 mmol) and cis-2-amino-1-cyclopentanecarboxylic acid hydrochloride (138 mg, 0.75 mmol) were reacted according to GP2 to give 3.0 mg of the title compound (166JO46). MS (ESI) 349 (MH⁺). Purity for MH⁺ (UV/MS) 99/88.

### Example 133

### General Procedure 10 (GP10)

A zinc reagent (0.4 mmol) was added at room temperature to a solution of 8,5-Dichloro-5H-dibenzo[b,e][1,4]diazepine (160FE64) (53 mg, 0.2 mmol) and PdCl₂(PPh₃)₂ (9 mg, 0.02 mmol) in dry THF (1 ml). The reaction was shaken until complete conversion (1-16 h, TLC) and then quenched by the addition of aqueous saturated NH₄Cl. The resulting mixture was extracted twice with ether and the combined ethereal phases were washed with brine and dried over Na₂SO₄. Filtration followed by concentration at reduced pressure of the organic phase gave a crude product, which was purified using column chromatography (heptane:EtOAc-system).

### Example 134

### 8-Chloro-11-(4-fluorobenzyl)-5H-dibenzo[b,e][1,4]diazepine (160FE59)

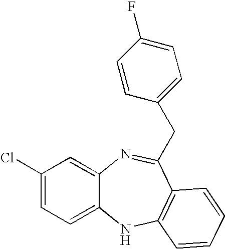

4-Fluorobenzylzinc chloride (0.8 ml, 0.5 M in THF, 0.4 mmol) and 8,5-dichloro-5H-dibenzo[b,e][1,4]diazepine (160FE64) (53 mg, 0.2 mmol) were reacted according GP10 to give 52 mg of the title compound (160FE59). MS (ESI) 337 (MH⁺). Purity for MH⁺ (UV/MS) 90/90.

### Example 134

### 8-Chloro-11-(4-fluorophenyl)-5H-dibenzo[b,e][1,4]diazepine (160FE70)

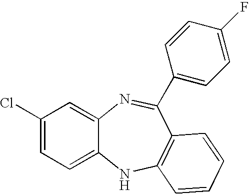

4-Fluorohenylzinc chloride (0.5 ml, 0.5 M in THF, 0.4 mmol) and 8,5-dichloro-5H-dibenzo[b,e][1,4]diazepine (160FE64) (26 mg, 0.1 mmol) were reacted according GP10 to give 23 mg of the title compound (160FE70). MS (ESI) 323 (MH⁺). Purity for MH⁺ (UV/MS) 98/100.

### Example 135

### General Procedure 11 (GP11)

Aqueous Na₂CO₃ (1 ml, 1M) was added at room temperature to a solution of the 8,5-dichloro-5H-dibenzo[b,e][1,4]diazepine (160FE64) (53 mg, 0.2 mmol) (26 mg, 0.1 mmol), Pd(PPh₃)₄ (10 mg), and the appropriate boronic acid reagent (0.12 mmol) in dioxane (3 ml). The mixture was then shaken at 80° C. until complete conversion of the imidoyl chloride (TLC). The temperature was decreased and ether and H₂O were added to the reaction mixture. The ether phase was washed with brine and dried over Na₂SO₄. Filtration followed by concentration at reduced pressure of the organic phase gave a crude product, which was purified using column chromatography (heptane:EtOAc-system).

### Example 136

### 8-Chloro-11-(4-nonylphenyl)-5H-dibenzo[b,e][1,4]diazepine (160FE63)

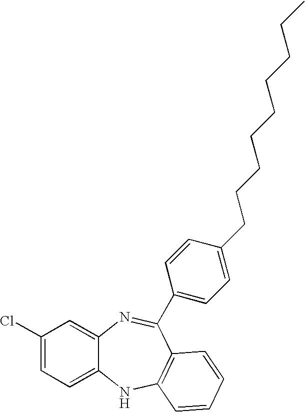

4-Nonylphenylboronic acid (30 mg, 0.12 mmol) and 8,5-dichloro-5H-dibenzo[b,e][1,4]diazepine (160FE64) (26 mg, 0.1 mmol) were reacted according GP11 to give 25 mg of the title compound (160FE63). MS (ESI) 431 (MH⁺). Purity for MH⁺ (UV/MS) 85/85.

### Example 137

### 8-Chloro-11-(pyridin-4-yl)-5H-dibenzo[b,e][1.4]diazepine (160FE69A

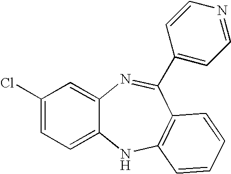

4 pyridyl-4-boronic acid (14 mg, 0.12 mmol) and 8,5-dichloro-5H-dibenzo[b,e][1,4]diazepine (160FE64) (26 mg, 0.1 mmol) were reacted according GP11 to give 9.3 mg of the title compound (160FE69A). MS (ESI) 306 (MH⁺). Purity for MH⁺ (UV/MS) 98/95.

### Example 138

### 8-Chloro-11-(1H-pyrazol-4-yl)-5H-dibenzo[b,e][1,4]diazepine (160FE59)

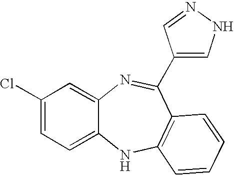

4-(4,4,5,5-Tetramethyl-1,3,2-dioxaborolan-2-yl)-1H-pyrazole (22 mg, 0.12 mmol) and 8,5-dichloro-5H-dibenzo[b,e][1,4]diazepine (160FE64) (26 mg, 0.1 mmol) were reacted according GP11 to give 8.7 mg of the title compound (160FE69B). MS (ESI) 295 (MH⁺). Purity for MH⁺ (UV/MS) 95/100.

### Example 139

### Activity of Known Antipsychotic Compounds

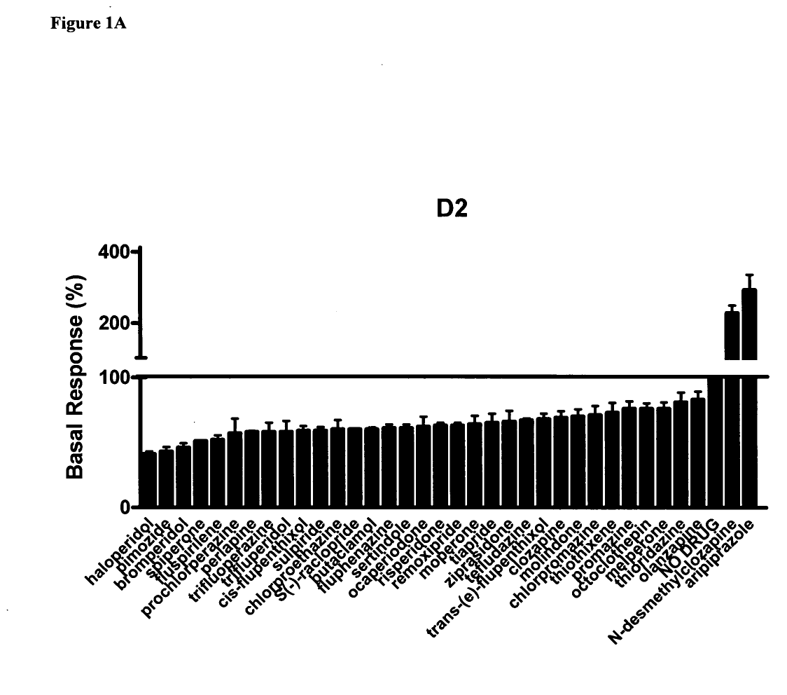

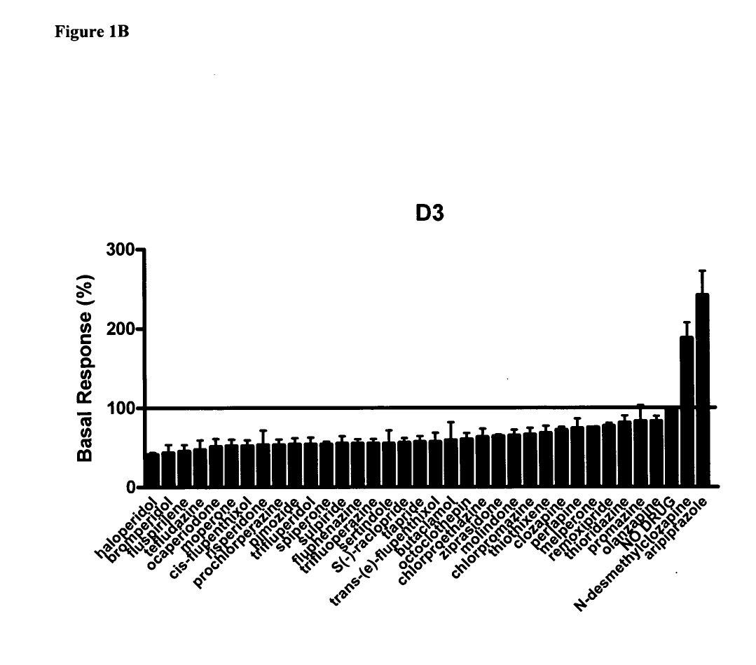

The functional receptor assay, Receptor Selection and Amplification Technology (R-SAT), was used (essentially as disclosed in U.S. Pat. No. 5,707,798, which is incorporated herein by reference in its entirety) with modifications described recently (29, 30), and with the additional modification that the G-protein Gαo was co-expressed with the human D2 and D3 dopaminergic receptors to induce constitutive activity of said receptors, to investigate the functional pharmacological properties of known antipsychotics, including many of their metabolites. Basal response was normalized to the basal response measured without any compounds included (i.e., no drug), which was assigned a value of 100%. Compounds were tested at 1 μM concentrations. The resulting basal activities at D2 and D3 receptors are presented in Table 1. The data in Table 1 represent the mean with the +/− indicating the standard error. These experiments provided a molecular profile, or fingerprint, for each of the agents. The results are also presented in order of increasing basal response in the bargraphs of FIG. 1A (D2 receptor) and FIG. 1B (D3 receptor).

TABLE 1
Instrinsic activites of antipsychotics at human
D2 and D3 dopamine receptors as
determined by R-SAT assays.

| Ligand | D2 basal | D3 basal |
|---|---|---|
| aripiprazole | 294 +/− 42 | 242 +/− 30 |
| N-desmethylclozapine | 229 +/− 21 | 188 +/− 20 |
| NO DRUG | 100− | 100− |
| olanzapine | 83 +/− 6 | 83 +/− 6 |
| thioridazine | 81 +/− 8 | 81 +/− 9 |
| melperone | 76 +/− 5 | 75− |
| octoclothepin | 76 +/− 4 | 60 +/− 8 |
| promazine | 76 +/− 6 | 83 +/− 20 |
| thiothixene | 73 +/− 7 | 68 +/− 9 |
| chlorpromazine | 71 +/− 7 | 66 +/− 8 |
| molindone | 70 +/− 6 | 65 +/− 7 |
| clozapine | 69 +/− 5 | 72 +/− 3 |
| trans-(e)-flupenthixol | 68 +/− 4 | 57 +/− 11 |
| tefludazine | 67 +/− 2 | 47 +/− 12 |
| ziprasidone | 66 +/− 8 | 64 +/− 2 |
| tiapride | 65 +/− 7 | 57 +/− 7 |
| moperone | 64 +/− 7 | 52 +/− 8 |
| remoxipride | 63 +/− 2 | 77 +/− 4 |
| risperidone | 63 +/− 2 | 53 +/− 18 |
| ocaperiodone | 62 +/− 8 | 51 +/− 10 |
| sertindole | 61 +/− 3 | 55 +/− 17 |
| fluphenazine | 61 +/− 3 | 55 +/− 5 |
| butaclamol | 60 +/− 2 | 59 +/− 22 |
| S(−)-raclopride | 60− | 56− |
| chlorproethazine | 60 +/− 7 | 63 +/− 10 |
| sulpiride | 59 +/− 3 | 55 +/− 9 |
| cis-flupenthixol | 59 +/− 4 | 52 +/− 7 |
| trifluperidol | 58 +/− 9 | 54 +/− 8 |
| trifluoperazine | 58 +/− 7 | 55 +/− 6 |
| perlapine | 58 +/− 0 | 74 +/− 12 |
| prochlorperazine | 57 +/− 11 | 53 +/− 7 |
| fluspirilene | 52 +/− 3 | 45 +/− 8 |
| spiperone | 51 +/− 1 | 54 +/− 4 |
| bromperidol | 46 +/− 3 | 43 +/− 10 |
| pimozide | 43 +/− 4 | 54 +/− 7 |
| haloperidol | 41 +/− 2 | 41 +/− 2 |

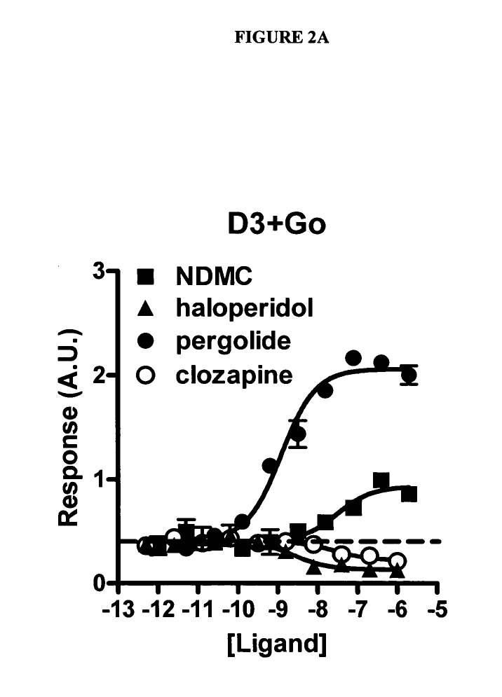

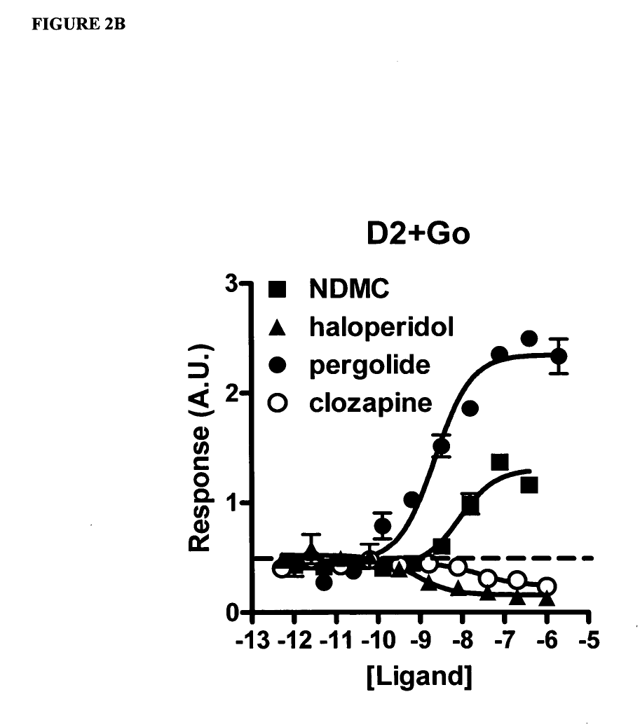

As illustrated in FIGS. 1A and 1B, of all of the agents tested, only aripiprazole and NDMC displayed D2 dopamine receptor agonist activity. The graphs in FIGS. 2A (D3 receptor) and 2B (D2 receptor) depict the concentration dependence of the receptor response to NDMC (filled squares), haloperidol (filled triangles), pergolide (filled circles), and clozapine (open circles). The dashed line in FIGS. 2A and 2B represents the basal activity in the absence of an added ligand. As illustrated in FIGS. 2A and 2B, clozapine displays high potency (pEC₅₀ of 7.2 and 7.6 at D2 and D3, respectively) yet displayed negative intrinsic efficacy at human D2 and D3 receptors. Clozapine is thus defined as an inverse agonist. Similarly, haloperidol was observed to be an inverse agonist at D2 and D3 receptors. Inverse agonists, besides acting as functional competitive antagonists of agonist action, reduce the intrinsic or agonist-independent activity of receptors (31), and may cause receptor upregulation/hypersensitization as previously shown for haloperidol at D2 receptors (21). In contrast, NDMC also displays high potency (pEC₅₀ of 7.5 and 7.0 at human D2 and D3 receptors, respectively), yet it displayed positive intrinsic activity at D2 and D3 receptors (34% and 40% relative efficacy to pergolide at D2 and D3, respectively), behaving as a partial agonist in the R-SAT.

### Example 140

### Dopamine Stabilizing Effect of NDMC

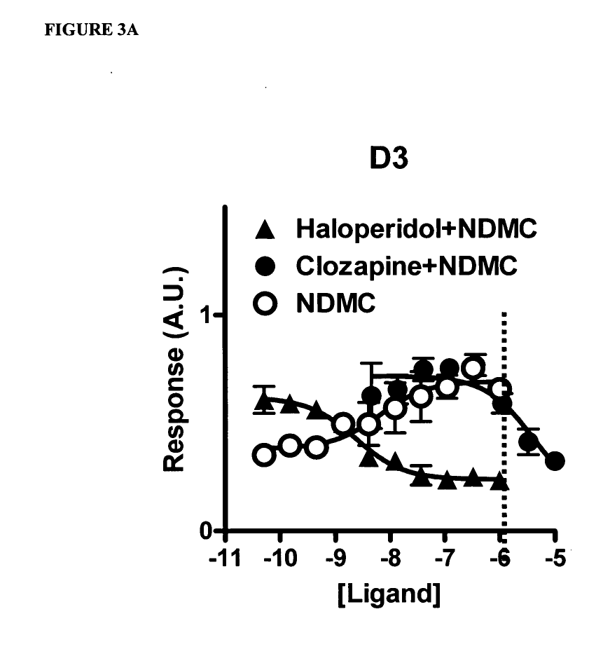

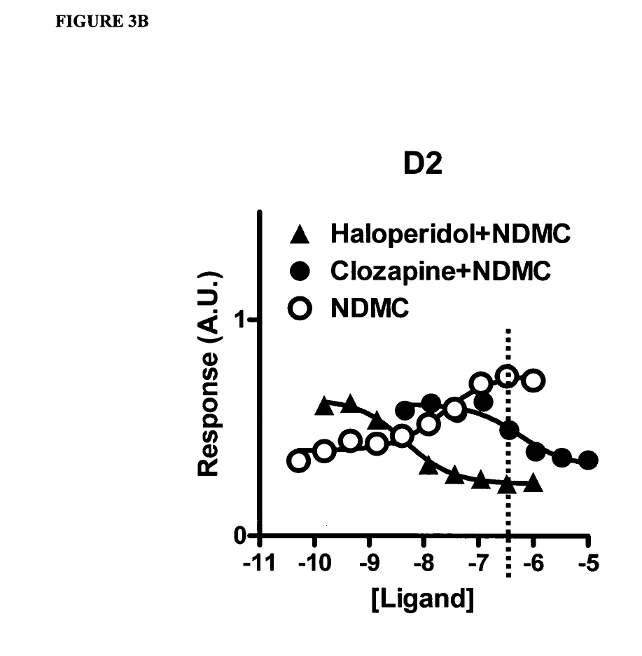

Clozapine and haloperidol were tested for their ability to block the agonist actions of NDMC at D2 and D3 dopaminergic receptors. The concentration response of NDMC in the R-SAT assay described in Example 1 was compared to the responses for haloperidol combined with NDMC and clozapine combined with NDMC. The response for the haloperidol and clozapine combinations was measured after each receptor was incubated with 300 nM NDMC. The concentration response curves are depicted in FIGS. 3A (D3 receptor) and 3B (D2 receptor). As shown in FIGS. 3A and 3B, both clozapine and haloperidol block the actions of the partial agonist NDMC at D2 and D3 dopaminergic receptors.

The doted lines in FIGS. 3A and 3B indicate the maximum concentrations of clozapine for which the combined NDMC and clozapine still exhibit a net agonism at the indicated receptors. For D2 receptors, the minimum NDMC to clozapine ratio for which a net agonism was observed was approximately 1:1 (FIG. 3B). For D3 receptors, the minimum NDMC to clozapine ratio for which a net agonism was observed was approximately 3:1 (FIG. 3A).

This positive efficacy suggests that NDMC will act as a partial agonist/competitive antagonist in vivo, a functional profile distinct from that observed for clozapine and most other antipsychotics that have negative intrinsic efficacy and that act as inverse agonists in vivo. These functional differences suggest that NDMC may act as a ‘dopamine stabilizer’/D2 stabilizer and have a lower propensity to cause extra pyramidal symptoms (EPS) and tardive dyskinesias (TDs) (15, 16), providing relief from these side effects, whereas most other antipsychotics will cause upregulation/hypersensitization of D2-like receptors in vivo due to their negative intrinsic activity at D2-like receptors (21), a phenomenon that has been associated with causing a predisposition towards EPS and TD.

### Example 141

### Dopamine Activity of NDMC Analogs

Various NDMC analogs described herein were subjected to a competitive radioligand D2 binding assay. The experiments were conducted on cell membranes harvested from HEK-293T cells transiently transfected with human D2 receptors. (Methoxy-³H)-raclopride competition curves using butaclamol as an experimental control were constructed and IC₅₀ values were determined using non-linear curve fitting. pKᵢ values were determined from the mean of one or two experiments. Basal response was normalized to the basal response measured without any compounds included (i.e., no drug), which was assigned a value of 100%. The results are depicted in Table 2 indicating that these compounds have intrinsic agonism or partial agonism at D2 receptors.

TABLE 2
R-SAT assay results indicating D2 intrinsic activity of NDMC analogs.

| | | % basal |
|---|---|---|
| COMPOUND | pKi | response |
| 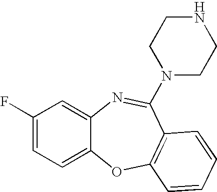 | 6.3 | 207 |
| 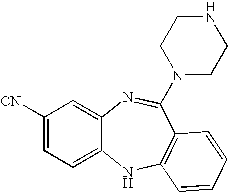 | 5.3 | 201 |
| 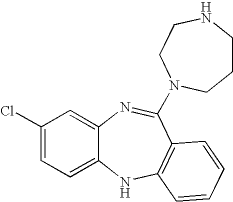 | 5.7 | 172 |
| 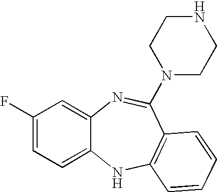 | 5.6 | 170 |
| 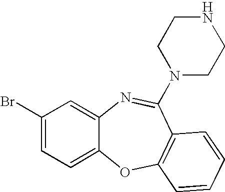 | 6.9 | 155 |
| 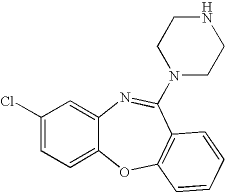 | 6.6 | 150 |
| 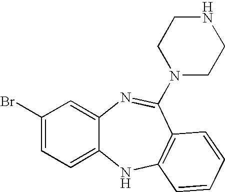 | 6.8 | 147 |
| 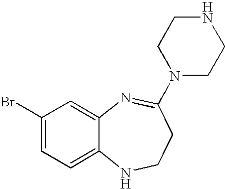 | NA | 142 |
| 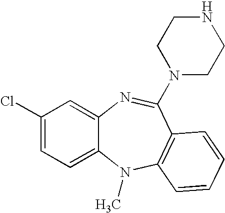 | 6.1 | 141 |
| 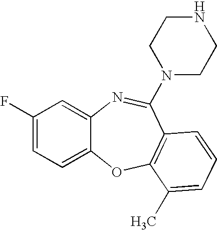 | 6.5 | 126 |
| 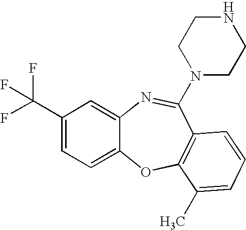 | 5.6 | 118 |
| 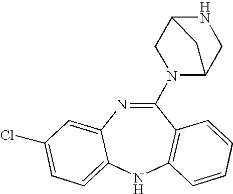 | 5.6 | 116 |
| 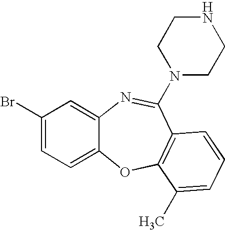 | 6.1 | 112 |
| 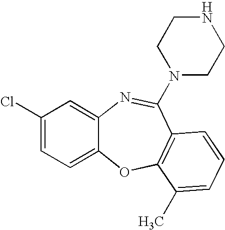 | 6.5 | 106 |
| 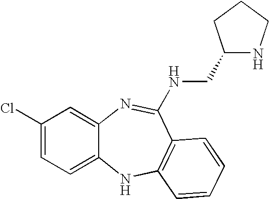 | 5.8 | 105 |
| 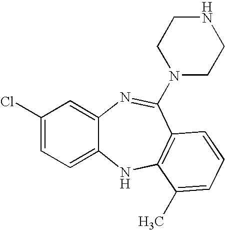 | 5.2 | 102 |
| 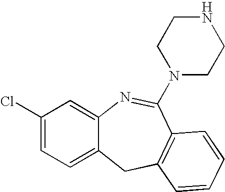 | 7.9 | 260 |

Although the invention has been described with reference to embodiments and examples, it should be understood that numerous and various modifications can be made without departing from the spirit of the invention. Accordingly, the invention is limited only by the following claims.

### REFERENCES

The following references are cited throughout this application by reference to the indicated numerals. Each such reference is incorporated herein by reference in their entirety.
1. Baumeister A A, Francis J L. Historical development of the dopamine hypothesis of schizophrenia. J Hist Neurosci. 2002 September; 11(3):265-77.
2. Strange P G. Antipsychotic drugs: importance of dopamine receptors for mechanisms of therapeutic actions and side effects. Pharmacol Rev. 2001 March; 53(1):119-33.
3. Creese I, Burt D R and Snyder S H (1976) Dopamine receptor binding predicts clinical and pharmacological potencies of antischizophrenic drugs. Science 192: 481-483.
4. Seeman P, Lee T, Chau-Wong M and Wong K (1976) Antipsychotic drug doses and neuroleptic/dopamine receptors. Nature 261: 717-719.
5. Meltzer H Y. What's atypical about atypical antipsychotic drugs? Curr Opin Pharmacol. 2004 4(1):53-7.
6. Hagger C, Buckley P, Kenny J T, Friedman L, Ubogy D, Meltzer H Y. Improvement in cognitive functions and psychiatric symptoms in treatment-refractory schizophrenic patients receiving clozapine. Biol Psychiatry. 1993 Nov. 15; 34(10):702-12.
7. Serretti A, De Ronchi D, Lorenzi C, Berardi D. New antipsychotics and schizophrenia: a review on efficacy and side effects. Curr Med. Chem. 2004 February; 11(3):343-58.
8. Shapleske J, Mickay A P, Mckenna P J. Successful treatment of tardive dystonia with clozapine and clonazepam. Br J Psychiatry. 1996 April; 168(4):516-8.
9. Trugman J M, Leadbetter R, Zalis M E, Burgdorf R O, Wooten G F. Treatment of severe axial tardive dystonia with clozapine: case report and hypothesis. Mov Disord. 1994 July; 9(4):441-6.
10. Charfi F, Cohen D, Houeto J L, Soubrie C, Mazet P. Tardive dystonia induced by atypical neuroleptics: a case report with olanzapine. J Child Adolesc Psychopharmacol. 2004 Spring; 14(1):149-52.
11. Casey D E. Tardive dyskinesia: pathophysiology and animal models. J Clin Psychiatry 2000; 61 Suppl 4:5-9.
12. Hall D A, Strange P G Evidence that antipsychotic drugs are inverse agonists at D2 dopamine receptors. Br J Pharmacol. 1997 June; 121(4):731-6.
13. Daeffler L, Landry Y. Inverse agonism at heptahelical receptors: concept, experimental approach and therapeutic potential. Fundam Clin Pharmacol. 2000 March-April;14(2):73-87.
14. Milligan G, MacEwan D J, Mercouris M, Mullaney I. Inverse agonism at adrenergic and opioid receptors: studies with wild type and constitutively active mutant receptors. Receptors Channels. 1997; 5(3-4):209-13.
15. Tamminga C A. Partial dopamine agonists in the treatment of psychosis. J Neural Transm. 2002 March; 109(3):411-20.
16. Tamminga C A, Carlsson A. Partial dopamine agonists and dopaminergic stabilizers, in the treatment of psychosis. Curr Drug Targets CNS Neurol Disord. 2002 April; 1(2):141-7.
17. Burris K D, Molski T F, Xu C, Ryan E, Tottori K, Kikuchi T, Yocca F D, Molinoff P B. Aripiprazole, a novel antipsychotic, is a high-affinity partial agonist at human dopamine D2 receptors. J Pharmacol Exp Ther. 2002 July; 302(1):381-9.
18. Lawler C P, Prioleau C, Lewis M M, Mak C, Jiang D, Schetz J A, Gonzalez A M, Sibley D R, Mailman R B. Interactions of the novel antipsychotic aripiprazole (OPC-14597) with dopamine and serotonin receptor subtypes. Neuropsychopharmacology. 1999 June; 20(6):612-27.
19. Shapiro D A, Renock S, Arrington E, Chiodo L A, Liu L X, Sibley D R, Roth B L, Mailman R. Aripiprazole, a novel atypical antipsychotic drug with a unique and robust pharmacology. Neuropsychopharmacology. 2003 August; 28(8):1400-11
20. Harrison T S & Perry C M Aripirazole: A review of its use in schizophrenia and schizoaffective disorder. Drugs 2004 64(15):1715-1736.
21. Inoue T, Domae M, Yamada K, Furukawa T. Effects of the novel antipsychotic agent 7-(4-[4-(2,3-dichlorophenyl)-1-piperazinyl]butyloxy)-3,4-dihydro-2(1H)-quinolinone (OPC-14597) on prolactin release from the rat anterior pituitary gland. J Pharmacol Exp Ther. 1996 April; 277(1):137-43.
22. Jann, M., W., Grimsley, S., R., Gray, E., C., and Chang, W. (1993) Pharmacokinetic and pharmacodynamics of clozapine. Clin. Pharmacokinet. 24(2): 161-176.
23. Bondesson, U., and Lindstrom. L., H., (1988) Determination of clozapine and its N-desmethylated metabolite in plasma by use of gas chromatography-mass spectrometry with single ion detection. Psychopharmacology. 95: 472-475.
24. Centorrino, F., Baldessarini, R., J., Kando, J., C., et. al. (1994) Clozapine and metabolites: concentrations in serum and clinical findings during treatment of chronically psychotic patients. J. Clin. Psychopharmacol. 14: 119-125.
25. Baldessarini, R., J., Centorrino, F., Flood, J., G., et. al. (1993) Tissue concentrations of clozapine and its metabolites in the rat. Neuropsychopharmacology. 9(2): 117-124.
26. Weigmann, H., Hartter, S., Fischer, V., Dahmen, N., and Hienike, C. (1999) Distribution of clozapine and desmethylclozapine between blood and brain in rats. European Neuropharmacology 9: 253-256.
27. Kuoppamaki, M., Syvalahti, E., and Hietala, J. (1993) Clozapine and N-desmethylclozapine are potent 5-HT1C receptor antagonists. Eur. J. Pharm. 245: 179-182.
28. Hunziker F. Fisher, E., and Scmutz, J. (1967) 11-amino-5H-dibenzo[b,e]-1,4-diazepine. Mitteilung uber siebenglienrige Heterocyclen. Helv. Chim. Acta, 50:1588-1599.
29. Weissman J T, Ma J N, Essex A, Gao Y, Burstein E S. G-protein-coupled receptor-mediated activation of rap GTPases: characterization of a novel Galphai regulated pathway. Oncogene. 2004 Jan. 8; 23(1):241-9.
30. Ma J N, Currier E A, Essex A, Feddock M, Spalding T A, Nash N R, Brann M R, Burstein E S. Discovery of novel peptide/receptor interactions: identification of PHM-27 as a potent agonist of the human calcitonin receptor. Biochem Pharmacol. 2004 Apr. 1; 67(7): 1279-84.
31. Hall D A, Strange P G Evidence that antipsychotic drugs are inverse agonists at D2 dopamine receptors. Br J Pharmacol. 1997 June; 121(4):731-6.

What is claimed is:

1. A method of ameliorating Extrapyramidal symptoms (EPS) and/or tardive dyskinesias (TD), comprising:
identifying a subject exhibiting Extrapyramidal symptoms (EPS) and/or tardive dyskinesias (TD); and
administering to the subject a therapeutically effective amount of a compound of Formula I, II, or XV: 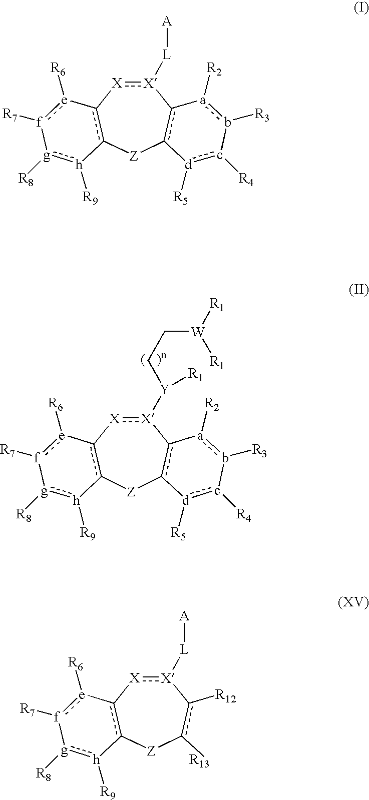
or a pharmaceutically acceptable salt, ester, amide, or prodrug thereof, wherein:
A is selected from the group consisting of 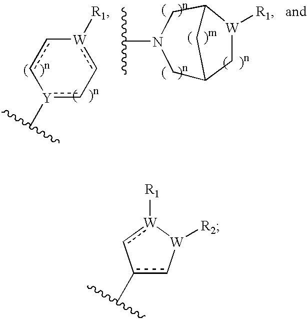
X is nitrogen, CH, or CH₂;
X′ is C or CH, wherein when X′ is C, there is a double bond between X and X′ and wherein when X′ is CH, there is a single bond between X and X′;
each Y is separately selected from the group consisting of nitrogen, oxygen, or CH;
each W is separately selected from the group consisting of nitrogen, CH, oxygen, or sulfur;
each n is separately selected from the group consisting of 0, 1, 2, 3, and 4;
m is selected from the group consisting of 1, 2, and 3;
each R₁ is separately absent or is separately selected from the group consisting of hydrogen, halogen, amine, optionally substituted C₁₋₂₀ alkyl, optionally substituted C₃₋₈ cycloalkyl, optionally substituted C₂₋₂₀ alkenyl, optionally substituted C₂₋₂₀ alkynyl, optionally substituted C₁₋₂₀-alkoxyalkyl, and optionally substituted aryl and arylalkyl;
L is absent or is selected from the group consisting of —NH(CH₂)ₙ— and —(CH₂)ₙ—;
a, b, c, and d are each separately selected from the group consisting of carbon, nitrogen, oxygen, and sulfur, or each is separately absent,
provided that at least three of a, b, c, or d are present,
provided that at least one of a, b, c, or d is carbon, and
provided that no two adjacent a, b, c, or d are both oxygen or both sulfur;
e, f, g, and h are each separately selected from the group consisting of carbon, nitrogen, oxygen, and sulfur, or each is separately absent,
provided that at least three of e, f, g, or h are present,
provided that at least one of e, f, g, or h is carbon, and
provided that no two adjacent e, f, g, or h are both oxygen or both sulfur;
R₂, R₃, R₄, and R₅, are each separately selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₂ and R₃, or R₃ and R₄, or R₄ and R₅ taken together, along with the ring carbons to which they are attached, form a five-membered or six-membered cycloalkyl, heterocyclyl or heteroaryl ring, or a six-membered aryl ring moiety;
R₆, R₇, R₈, and R₉, are each separately selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₆ and R₇, or R₇ and R₈, or R₈ and R₉ taken together, along with the ring carbons to which they are attached, form a five-membered or six-membered cycloalkyl, heterocyclyl or heteroaryl ring, or a six-membered aryl ring moiety;
Z is selected from the group consisting of NR₁₁, oxygen, sulfur, and CH₂;
R₁₀ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl optionally substituted aryl, optionally substituted arylalkyl, and perhaloalkyl; and
R₁₁ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl, and optionally substituted arylalkyl;
R₁₂ and R₁₃ are separately selected from the group consiting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₁₂ and R₁₃, taken together, along with the ring carbons to which they are attached, form a five-membered or six-membered cycloalkyl, heterocyclyl or heteroaryl ring, or a six-membered aryl ring moiety;
any bond represented by a dashed and solid line represents a bond selected from the group consisting of a carbon-carbon single bond and a carbon-carbon double bond.

2. The method of claim 1, wherein said compound has a structure set forth in Formulas III or IV.

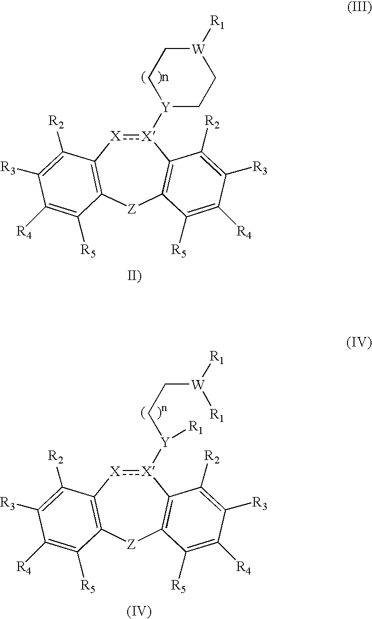

3. The method of claim 1, wherein said compound is selected from the group consisting of:

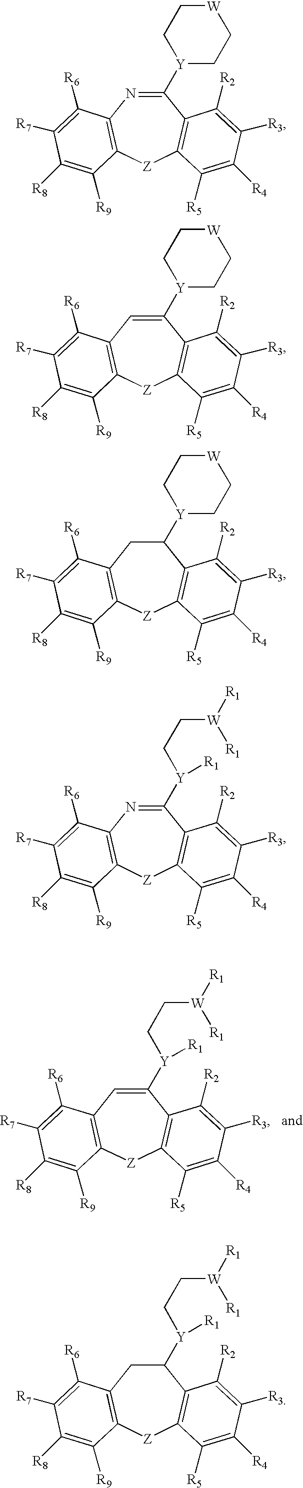

4. The method of claim 3, wherein said compound is selected from the group consisting of:

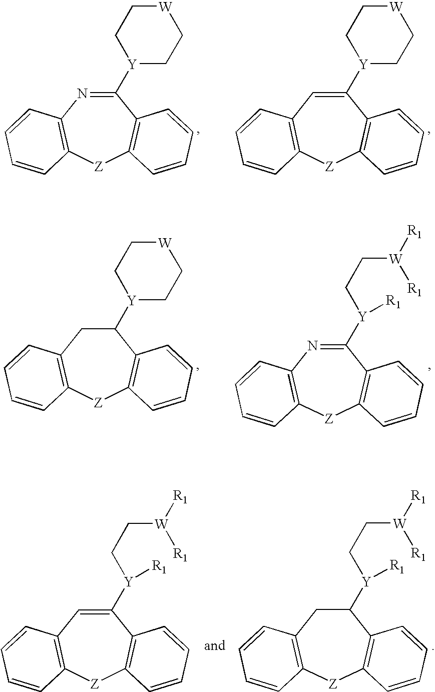

5. The method of claim 1, wherein none of a, b, c, or d is absent.

6. The method of claim 1, wherein none of e, f, g, or h is absent.

7. The method of claim 1, wherein a, b, c, and d are carbon.

8. The method of claim 1, wherein e, f, g, and h are carbon.

9. The method of claim 1, wherein R₂ is selected from the group consisting of hydrogen, halogen, optionally substituted C₁₋₆ alkyl, and optionally substituted C₁₋₆ alkyloxy.

10. The method of claim 1, wherein R₂ is selected from the group consisting of hydrogen, methyl, methoxy, and chloro.

11. The method of claim 1, wherein R₃ is selected from the group consisting of hydrogen, halogen, optionally substituted C₁₋₆ alkyl, optionally substituted C₁₋₆ alkyloxy, and NO₂

12. The method of claim 1, wherein R₃ is selected from the group consisting of hydrogen, methyl, methoxy, chloro, bromo, iodo, and NO₂.

13. The method of claim 1, wherein R₄ is selected from the group consisting of hydrogen, halogen, optionally substituted C₁₋₆ alkyl, perhaloalkyl, SO₂R₁₀, and NO₂.

14. The method of claim 1, wherein R₅ is selected from the group consisting of hydrogen, halogen, and optionally substituted C₁₋₆ alkyl.

15. The method of claim 1, wherein R₅ is hydrogen or chloro.

16. The method of claim 1, wherein R₆ is hydrogen or optionally substituted C₁₋₆ alkyl.

17. The method of claim 1, wherein R₇ is selected from the group consisting of hydrogen, halogen, optionally substituted C₁₋₆ alkyl, perhaloalkyl, CN, SO₂R₁₀, and NO₂.

18. The method of claim 1, wherein R₈ is selected from the group consisting of hydrogen, halogen, optionally substituted C₁₋₆ alkyl.

19. The method of claim 1, wherein R₈ is selected from the group consisting of hydrogen, chloro, and bromo.

20. The method of claim 1, wherein R₉ is selected from the group consisting of hydrogen, halogen, optionally substituted C₁₋₆ alkyl, and perhaloalkyl.

21. The method of claim 1, wherein R₉ is selected from the group consisting of hydrogen, chloro, methyl, and trifluoromethyl.

22. The method of claim 1, wherein R₁ is selected from the group consisting of hydrogen, optionally substituted C₁₋₆ alkyl, and optionally substituted aryl.

23. The method of claim 1, wherein R₁ is hydrogen.

24. The method of claim 1, wherein X is nitrogen.

25. The method of claim 1, wherein Y is NH.

26. The method of claim 1, wherein L is absent or is selected from the group consisting of —NHCH₂—, —NH—, and —CH₂—.

27. The method of claim 1, wherein A is selected from the group consisting of: 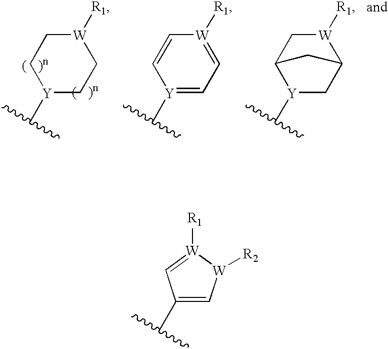
and wherein n is selected from the group consiting of 0, 1, and 2.

28. The method of claim 1, wherein the compound is selected from the group consiting of:
2,7-Dichloro-11-(piperazin-1-yl)-5H-dibenzo[b,e][1,4]diazepine,
2-Chloro-11-(piperazin-1-yl)-5H-dibenzo[b,e][1,4]diazepine,
2,8-Dichloro-11-(piperazin-1-yl)-5H-dibenzo[b,e][1,4]diazepine,
8-Bromo-2-chloro-11-(piperazin-1-yl)-5H-dibenzo[b,e][1,4]diazepine,
2-Chloro-11-(piperazin-1-yl)-8-trifluoromethyl-5H-dibenzo[b,e][1,4]diazepine,
6-Chloro-11-(piperazin-1-yl)-8-trifluoromethyl-5H-dibenzo[b,e][1,4]diazepine,
7-Chloro-11-(piperazin-1-yl)-5H-dibenzo[b,e][1,4]diazepine,
8-Bromo-1-chloro-11-(piperazin-1-yl)-5H-dibenzo[b,e][1,4]diazepine,
8-Bromo-2-methyl-11-(piperazin-1-yl)-5H-dibenzo[b,e][1,4]diazepine,
4,8-Dichloro-11-(piperazin-1-yl)-5H-dibenzo[b,e][1,4]diazepine,
8-Chloro-2-methyl-11-(piperazin-1-yl)-5H-dibenzo[b,e][1,4]diazepine,
8-Chloro-2-fluoro-11-(piperazin-1-yl)-5H-dibenzo[b,e][1,4]diazepine,
3,8-Dichloro-11-(piperazin-1-yl)-5H-dibenzo[b,e][1,4]diazepine,
2-Bromo-8-chloro-11-(piperazin-1-yl)-5H-dibenzo[b,e][1,4]diazepine,
3,7-Dichloro-11-(piperazin-1-yl)-5H-dibenzo[b,e][1,4]diazepine,
8-Bromo-3-chloro-11-(piperazin-1-yl)-5H-dibenzo[b,e][1,4]diazepine,
3-Chloro-11-(piperazin-1-yl)-5H-dibenzo[b,e][1,4]diazepine,
3-Chloro-11-(piperazin-1-yl)-8-trifluoromethyl-5H-dibenzo[b,e][1,4]diazepine,
7-Chloro-2-methyl-11-(piperazin-1-yl)-5H-dibenzo[b,e][1,4]diazepine,
2-Methyl-11-(piperazin-1-yl)-5H-dibenzo[b,e][1,4]diazepine,
2-Methyl-11-(piperazin-1-yl)-8-trifluoromethyl-5H-dibenzo[b,e][1,4]diazepine,
8-Chloro-4-methyl-11-(piperazin-1-yl)-5H-dibenzo[b,e][1,4]diazepine,
1,8-Dichloro-11-(piperazin-1-yl)-5H-dibenzo[b,e][1,4]diazepine,
8-Bromo-5-methyl-11-(piperazin-1-yl)-5H-dibenzo[b,e][1,4]diazepine,
7,8-Dichloro-11-(piperazin-1-yl)-5H-dibenzo[b,e][1,4]diazepine,
11-(piperazin-1-yl)-8-trifluoromethyl-5H-dibenzo[b,e][1,4]diazepine,
11-(piperazin-1-yl)-5H-dibenzo[b,e][1,4]diazepine,
8-Fluoro-11-(piperazin-1-yl)-5H-dibenzo[b,e][1,4]diazepine,
11-(piperazin-1-yl)-5H-dibenzo[b,e][1,4]diazepine-8-carbonitrile,
8-Bromo-1-(piperazin-1-yl)-5H-dibenzo[b,e][1,4]diazepine,
8-Methyl-11-(piperazin-1-yl)-5H-dibenzo[b,e][1,4]diazepine,
3-Fluoro-6-piperazin-1-yl-11H-dibenzo[b,e]azepine,
2-(Trifluoromethanesulfonyloxy 11-(piperazin-1-yl)-5H-dibenzo[b,e][1,4]diazepine,
2-(Trifluoromethanesulfonyloxy)-11-(piperazin-1-yl)-5H-dibenzo[b,e][1,4]oxazepine,
8-Chloro-2-(trifluoromethanesulfonyloxy)-11-(piperazin-1-yl)-5H-dibenzo[b,e][1,4]diazepine,
8-(Trifluoromethanesulfonyloxy)-11-(piperazin-1-yl)-5H-dibenzo[b,e][1,4]diazepine,
11-(piperazin-1-yl)-dibenzo[b,f][1,4]thiazepin, 11-(piperazin-1-yl)-2,3-dihydro-1,4-benzodioxino[6,7-b][1,4]benzothiazepin,
8-Chloro-1-[1,4]diazepam-1-yl-5H-dibenzo[b,e][1,4]diazepine,
N′-(8-Chloro-5H-dibenzo[b,e][1,4]diazepine-11-yl)-N,N-dimethyl-ethane-1,2-diamine,
N′-(8-Chloro-5H-dibenzo[b,e][1,4]diazepine-11-yl)-N,N-diethyl-ethane-1,2-diamine,
8-Chloro-11-(4-methyl-[1,4]diazepam-1-yl)-5H-dibenzo[b,e][1,4]diazepine,
8-Chloro-2-methoxy-11-(piperazin-1-yl)-5H-dibenzo[b,e][1,4]diazepine,
N′-(5H-Dibenzo[b,e][1,4]diazepine-11-yl)-N,N-dimethyl-ethane-1,2-diamine,
11-[1,4]Diazepam-1-yl-5H-dibenzo[b,e][1,4]diazepine,
N′-(8-Fluoro-5H-dibenzo[b,e][1,4]diazepine-11-yl)-N,N-dimethyl-ethane-1,2-diamine,
8-Fluoro-11-[1,4]diazepam-1-yl-5H-dibenzo[b,e][1,4]diazepine,
N′-(8-Chloro-5H-dibenzo[b,e][1,4]diazepine-11-yl)-N-methyl-ethane-1,2-diamine,
8-Chloro-11-(trans-2,5-dimethyl-piperazin-1-yl)-5H-dibenzo[b,e][1,4]diazepine,
8-Chloro-11-(3,5-dimethyl-piperazin-1-yl)-5H-dibenzo[b,e][1,4]diazepine,
8-Chloro-11-(3-methyl-piperazin-1-yl)-5H-dibenzo[b,e][1,4]diazepine,
8-Chloro-11-(3-phenyl-piperazin-1-yl)-5H-dibenzo[b,e][1,4]diazepine,
8-Chloro-5-methyl-11-(piperazin-1-yl)-5H-dibenzo[b,e][1,4]diazepine,
8-Chloro-5-benzyl-11-(piperazin-1-yl)-5H-dibenzo[b,e][1,4]diazepine,
8-Iodo-11-(piperazin-1-yl)-5H-dibenzo[b,e][1,4]diazepine,
2-Iodo-8-chloro-11-(piperazin-1-yl)-5H-dibenzo[b,e][1,4]diazepine,
8-Phenyl-11-(piperazin-1-yl)-5H-dibenzo[b,e][1,4]diazepine,
8-Chloro-11-(piperidin-1-yl)-5H-dibenzo[b,e][1,4]diazepine,
8-Chloro-11-(morpholin-4-yl)-5H-dibenzo[b,e][1,4]diazepine,
5-Allyl-8-chloro-11-(piperazin-1-yl)-5H-dibenzo[b,e][1,4]diazepine,
6-Chloro-11-(piperazin-1-yl)-5H-dibenzo[b,e][1,4]diazepine,
8-Chloro-5-piperazin-1-yl-11H-benzo[b]pyrido[2,3-e][1,4]diazepine,
2-Chloro-10-piperazin-1-yl-5H-dibenzo[b,f]azepin,
8-Chloro-11-(piperazin-1-yl)-dibenzo[b,f][1,4]thiazepine,
8-Chloro-11-(piperazin-1-yl)-dibenzo[b,f][1,4]oxazepine,
8-Chloro-11-(4-methyl-piperazin-1-yl)-dibenzo[b,f][1,4]oxazepine,
3-Chloro-6-piperazin-1-yl-11H-dibenzo[b,e]azepine,
8-Bromo-11-(piperazin-1-yl)-dibenzo[b,f][1,4]oxazepine,
11-(piperazin-1-yl)-dibenzo[b,f][1,4]oxazepine,
7-Chloro-11-(piperazin-1-yl)-dibenzo[b,f][1,4]oxazepine,
8-Chloro-3-methoxy-11-(piperazin-1-yl)-dibenzo[b,f][1,4]oxazepine,
8-Bromo-3-methoxy-11-(piperazin-1-yl)-dibenzo[b,f][1,4]oxazepine,
3-Methoxy-11-(piperazin-1-yl)-dibenzo[b,f][1,4]oxazepine,
7-Chloro-3-methoxy-11-(piperazin-1-yl)-dibenzo[b,f][1,4]oxazepine,
8-Chloro-4-methyl-11-(piperazin-1-yl)-dibenzo[b,f][1,4]oxazepine,
8-Bromo-4-methyl-11-(piperazin-1-yl)-dibenzo[b,f][1,4]oxazepine,
4-Methyl-11-(piperazin-1-yl)-dibenzo[b,f][1,4]oxazepine,
2-Bromo-8-chloro-11-(piperazin-1-yl)-dibenzo[b,f][1,4]oxazepine,
2,8-Dibromo-11-(piperazin-1-yl)-dibenzo[b,f][1,4]oxazepine,
2-Bromo-11-(piperazin-1-yl)-dibenzo[b,f][1,4]oxazepine,
2-Bromo-7-chloro-11-(piperazin-1-yl)-dibenzo[b,f][1,4]oxazepine,
11-(piperazin-1-yl)-8-trifluoromethyl-dibenzo[b,f][1,4]oxazepine,
4-Methyl-11-(piperazin-1-yl)-8-trifluoromethyl-dibenzo[b,f][1,4]oxazepine,
8-Fluoro-11-(piperazin-1-yl)-dibenzo[b,f][1,4]oxazepine,
8-Fluoro-3-methoxy-11-(piperazin-1-yl)-dibenzo[b,f][1,4]oxazepine,
8-Fluoro-4-methyl-11-(piperazin-1-yl)-dibenzo[b,f][1,4]oxazepine,
2-Bromo-8-fluoro-11-(piperazin-1-yl)-dibenzo[b,f][1,4]oxazepine,
8-Methyl-11-(piperazin-1-yl)-dibenzo[b,f][1,4]oxazepine,
3-Methoxy-8-methyl-11-(piperazin-1-yl)-dibenzo[b,f][1,4]oxazepine,
4,8-Dimethyl-11-(piperazin-1-yl)-dibenzo[b,f][1,4]oxazepine,
3-Methoxy-11-(piperazin-1-yl)-8-trifluoromethyl-dibenzo[b,f][1,4]oxazepine,
2-Bromo-11-(piperazin-1-yl)-8-trifluoromethyl-dibenzo[b,f][1,4]oxazepine,
6-Chloro-11-(piperazin-1-yl)-dibenzo[b,f][1,4]oxazepine,
2-Bromo-8-methyl-11-(piperazin-1-yl)-dibenzo[b,f][1,4]oxazepine,
7-Chloro-4-methyl-11-(piperazin-1-yl)-dibenzo[b,f][1,4]oxazepine,
8-Phenyl-11-(piperazin-1-yl)-dibenzo[b,f][1,4]oxazepine,
8-Chloro-11-(piperidin-4-yl)-5H-dibenzo[b,e][1,4]diazepine
5-Benzyl-8-chloro-11-(piperidin-4-yl)-5H-dibenzo[b,e][1,4]diazepine,
8-Bromo-5,10-dihydro-dibenzo[b,e][1,4]diazepine-11-one,
5,10-Dihydro-dibenzo[b,e][1,4]diazepine-11-one,
8-Fluoro-5,10-dihydro-dibenzo[b,e][1,4]diazepine-11-one,
8,5-Dichloro-5H-dibenzo[b,e][1,4]diazepine,
8-Chloro-11-methylsulfanyl-5H-dibenzo[b,e][1,4]diazepine
(8-Chloro-5H-dibenzo[b,e][1,4]diazepin-11-yl)-(S)-1-pyrrolidin-2-yl-methyl-amine,
1-(8-Chloro-5H-dibenzo[b,e][1,4]diazepin-11-yl)-piperidine-4-yl-amine,
1-(8-Chloro-5H-dibenzo[b,e][1,4]diazepin-11-yl)-pyrrolidin-3-yl-amine,
(8-Chloro-5H-dibenzo[b,e][1,4]diazepin-11-yl)-(R)-1-pyrrolidin-2-yl-methyl-amine,
(8-Chloro-5H-dibenzo[b,e][1,4]diazepin-11-yl)-pyrrolidin-3-yl-amine,
8-Chloro-11-(2,5-diaza-bicyclo[2.2.1]hept-2-yl)-5H-dibenzo[b,e][1,4]diazepine,
Acetidin-3-yl-8-chloro-5H-dibenzo[b,e][1,4]diazepine-11-yl)amine,
7-Bromo-4-(piperazin-1-yl)-2,3-dihydro-1H-benzo[b][1,4]diazepine,
7-Bromo-2-methyl-(piperazin-1-yl)-2,3-dihydro-1H-benzo[b][1,4]diazepine
7-Bromo-2-phenyl-4-(piperazine-1-yl)-2,3-dihydro-1H-benzo[b][1,4]diazepine,
7-Bromo-10-(piperazin-1-yl)-1,2,3,3a,4,10a-hexahydro-benzo[b]cyclopenta[e][1,4]diazepine,
8-Chloro-11-(4-fluorobenzyl)-5H-dibenzo[b,e][1,4]diazepine,
8-Chloro-11-(4-fluorophenyl)-5H-dibenzo[b,e][1,4]diazepine,
8-Chloro-11-(4-nonylphenyl)-5H-dibenzo[b,e][1,4]diazepine,
8-Chloro-11-(pyridin-4-yl)-5H-dibenzo[b,e][1,4]diazepine, and
8-Chloro-11-(1H-pyrazol-4-yl)-5H-dibenzo[b,e][1,4]diazepine.

29. The method of claim 1, where the compound is N-desmethylclozapine.

30. The method of claim 1, wherein the subject is human.

31. The method of claim 1, wherein the identifiying comprises identifying a subject exhibiting Extrapyramidal symptoms (EPS) and/or tardive dyskinesias (TD) as a result of exposure to one or more medications

32. A method of ameliorating Extrapyramidal symptoms (EPS) and/or tardive dyskinesias (TD), comprising administering to a subject exhibiting Extrapyramidal symptoms (EPS) and/or tardive dyskinesias (TD) a therapeutically effective amount of N-desmethylclozapine essentially free of clozapine.

33. The method of claim 32, wherein the subject exhibits Extrapyramidal symptoms (EPS) and/or tardive dyskinesias (TD) as a result of exposure to one or more medications.

34. A method of ameliorating Extrapyramidal symptoms (EPS) and/or tardive dyskinesias (TD), comprising administering to a subject exhibiting Extrapyramidal symptoms (EPS) and/or tardive dyskinesias (TD) a therapeutically effective amount of a pharmaceutical composition comprising N-desmethylclozapine and a pharmaceutically acceptable excipient or diluent, wherein the amount of any clozapine administered is low enough such that the combined N-desmethylclozapine and clozapine result in a net agonism at dopamine receptors.

35. The method of claim 34, wherein the subject exhibits Extrapyramidal symptoms (EPS) and/or tardive dyskinesias (TD) as a result of exposure to one or more medications.

36. A method of treating a subject refractory to other treatments due to a propensity to develop Extrapyramidal symptoms (EPS) and/or tardive dyskinesias (TD), comprising administering to a subject having a propensity to develop Extrapyramidal symptoms (EPS) and/or tardive dyskinesias (TD) a therapeutically effective amount of a compound of Formula I, II, or XV: 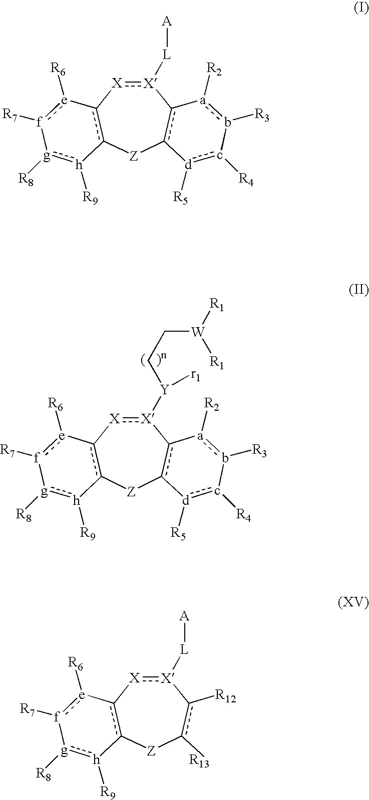
or a pharmaceutically acceptable salt, ester, amide, or prodrug thereof, wherein:
A is selected from the group consisting of 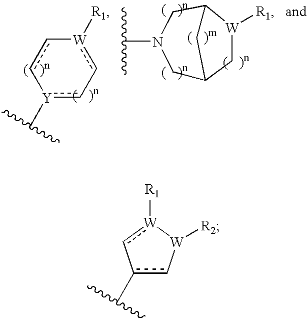
X is nitrogen, CH, or CH₂;
X′ is C or CH, wherein when X′ is C, there is a double bond between X and X′ and wherein when X′ is CH, there is a single bond between X and X′;
each Y is separately selected from the group consisting of nitrogen, oxygen, or CH;
each W is separately selected from the group consisting of nitrogen, CH, oxygen, or sulfur;
each n is separately selected from the group consisting of 0, 1, 2, 3, and 4;
m is selected from the group consisting of 1, 2, and 3;
each R₁ is separately absent or is separately selected from the group consisting of hydrogen, halogen, amine, optionally substituted C₁₋₂₀ alkyl, optionally substituted C₃₋₈ cycloalkyl, optionally substituted C₂₋₂₀ alkenyl, optionally substituted C₂₋₂₀ alkynyl, optionally substituted C₁₋₂₀-alkoxyalkyl, and optionally substituted aryl and arylalkyl;
L is absent or is selected from the group consisting of —NH(CH₂)ₙ— and —(CH₂)ₙ—;
a, b, c, and d are each separately selected from the group consisting of carbon, nitrogen, oxygen, and sulfur, or each is separately absent,
provided that at least three of a, b, c, or d are present,
provided that at least one of a, b, c, or d is carbon, and
provided that no two adjacent a, b, c, or d are both oxygen or both sulfur;
e, f, g, and h are each separately selected from the group consisting of carbon, nitrogen, oxygen, and sulfur, or each is separately absent,
provided that at least three of e, f, g, or h are present,
provided that at least one of e, f, g, or h is carbon, and
provided that no two adjacent e, f, g, or h are both oxygen or both sulfur;
R₂, R₃, R₄, and R₅, are each separately selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₂ and R₃, or R₃ and R₄, or R₄ and R₅ taken together, along with the ring carbons to which they are attached, form a five-membered or six-membered cycloalkyl, heterocyclyl or heteroaryl ring, or a six-membered aryl ring moiety;
R₆, R₇, R₈, and R₉, are each separately selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₆ and R₇, or R₇ and R₉, or R₈ and R₉ taken together, along with the ring carbons to which they are attached, form a five-membered or six-membered cycloalkyl, heterocyclyl or heteroaryl ring, or a six-membered aryl ring moiety;
Z is selected from the group consisting of NR₁₁, oxygen, sulfur, and CH₂;
R₁₀ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl optionally substituted aryl, optionally substituted arylalkyl, and perhaloalkyl; and
R₁₁ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl, and optionally substituted arylalkyl;
R₁₂ and R₁₃ are separately selected from the group consiting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₁₂ and R₁₃, taken together, along with the ring carbons to which they are attached, form a five-membered or six-membered cycloalkyl, heterocyclyl or heteroaryl ring, or a six-membered aryl ring moiety;
any bond represented by a dashed and solid line represents a bond selected from the group consisting of a carbon-carbon single bond and a carbon-carbon double bond.

37. The method of claim 36, further comprising identifying a subject having a propensity to develop Extrapyramidal symptoms (EPS) and/or tardive dyskinesias (TD).

38. The method of claim 36, wherein the compound is N-desmethylclozapine and the amount of any clozapine administered is low enough such that the combined N-desmethylclozapine and clozapine result in a net agonism at dopamine receptors.

39. A method of dopamine stabilization, comprising:
identifying a subject in need of dopamine stabilization; and
administering to the subject an amount of a compound of Formula I, II, or XV effective to stabilize one or more dopamine receptors: 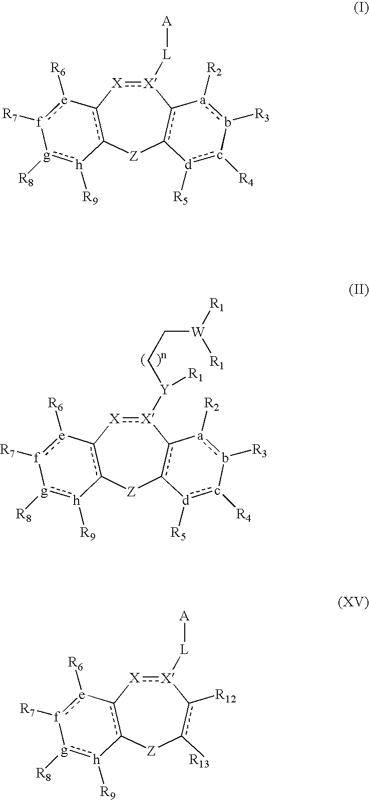
or a pharmaceutically acceptable salt, ester, amide, or prodrug thereof, wherein:
A is selected from the group consisting of 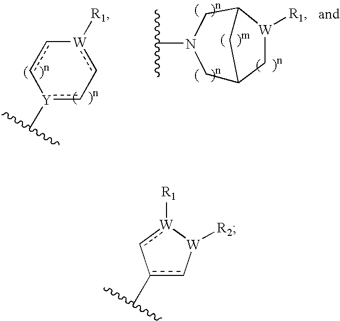
X is nitrogen, CH, or CH₂;
X′ is C or CH, wherein when X′ is C, there is a double bond between X and X′ and wherein when X′ is CH, there is a single bond between X and X′;
each Y is separately selected from the group consisting of nitrogen, oxygen, or CH;
each W is separately selected from the group consisting of nitrogen, CH, oxygen, or sulfur;
each n is separately selected from the group consisting of 0, 1, 2, 3, and 4;
m is selected from the group consisting of 1, 2, and 3;
each R₁ is separately absent or is separately selected from the group consisting of hydrogen, halogen, amine, optionally substituted C₁₋₂₀ alkyl, optionally substituted C₃₋₈ cycloalkyl, optionally substituted C₂₋₂₀ alkenyl, optionally substituted C₂₋₂₀ alkynyl, optionally substituted C₁₋₂₀-alkoxyalkyl, and optionally substituted aryl and arylalkyl;
L is absent or is selected from the group consisting of —NH(CH₂)ₙ— and —(CH₂)ₙ—;
a, b, c, and d are each separately selected from the group consisting of carbon, nitrogen, oxygen, and sulfur, or each is separately absent,
provided that at least three of a, b, c, or d are present,
provided that at least one of a, b, c, or d is carbon, and
provided that no two adjacent a, b, c, or d are both oxygen or both sulfur;
e, f, g, and h are each separately selected from the group consisting of carbon, nitrogen, oxygen, and sulfur, or each is separately absent,
provided that at least three of e, f, g, or h are present,
provided that at least one of e, f, g, or h is carbon, and
provided that no two adjacent e, f, g, or h are both oxygen or both sulfur;
R₂, R₃, R₄, and R₅, are each separately selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₂ and R₃, or R₃ and R₄, or R₄ and R₅ taken together, along with the ring carbons to which they are attached, form a five-membered or six-membered cycloalkyl, heterocyclyl or heteroaryl ring, or a six-membered aryl ring moiety;
R₆, R₇, R₈, and R₉, are each separately selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₆ and R₇, or R₇ and R₈, or R₈ and R₉ taken together, along with the ring carbons to which they are attached, form a five-membered or six-membered cycloalkyl, heterocyclyl or heteroaryl ring, or a six-membered aryl ring moiety;
Z is selected from the group consisting of NR₁₁, oxygen, sulfur, and CH₂;
R₁₀ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl optionally substituted aryl, optionally substituted arylalkyl, and perhaloalkyl; and
R₁₁ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl, and optionally substituted arylalkyl;
R₁₂ and R₁₃ are separately selected from the group consiting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₁₂ and R₁₃, taken together, along with the ring carbons to which they are attached, form a five-membered or six-membered cycloalkyl, heterocyclyl or heteroaryl ring, or a six-membered aryl ring moiety;
any bond represented by a dashed and solid line represents a bond selected from the group consisting of a carbon-carbon single bond and a carbon-carbon double bond.

40. The method of claim 39, wherein the dopamine receptor is a D2 receptor.

41. A method of modulating D2 receptors, comprising:
identifying a subject in need of D2 receptor modulation; and
contacting D2 receptors in the subject with a compound of Formula I, II, or XV: 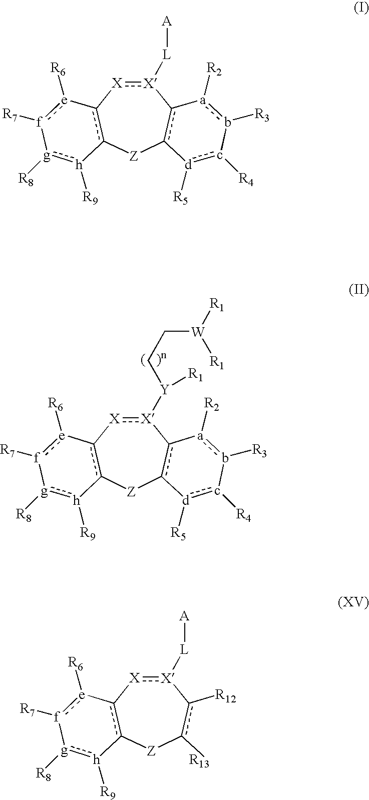
or a pharmaceutically acceptable salt, ester, amide, or prodrug thereof, wherein:
A is selected from the group consisting of 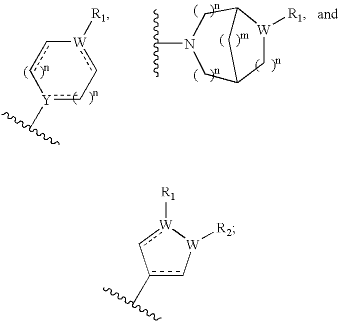
X is nitrogen, CH, or CH₂;
X′ is C or CH, wherein when X′ is C, there is a double bond between X and X′ and wherein when X′ is CH, there is a single bond between X and X′;
each Y is separately selected from the group consisting of nitrogen, oxygen, or CH;
each W is separately selected from the group consisting of nitrogen, CH, oxygen, or sulfur;
each n is separately selected from the group consisting of 0, 1, 2, 3, and 4;
m is selected from the group consisting of 1, 2, and 3;
each R₁ is separately absent or is separately selected from the group consisting of hydrogen, halogen, amine, optionally substituted C₁₋₂₀ alkyl, optionally substituted C₃₋₈ cycloalkyl, optionally substituted C₂₋₂₀ alkenyl, optionally substituted C₂₋₂₀ alkynyl, optionally substituted C₁₋₂₀-alkoxyalkyl, and optionally substituted aryl and arylalkyl;
L is absent or is selected from the group consisting of —NH(CH₂)ₙ— and —(CH₂)ₙ—;
a, b, c, and d are each separately selected from the group consisting of carbon, nitrogen, oxygen, and sulfur, or each is separately absent,
provided that at least three of a, b, c, or d are present,
provided that at least one of a, b, c, or d is carbon, and
provided that no two adjacent a, b, c, or d are both oxygen or both sulfur;
e, f, g, and h are each separately selected from the group consisting of carbon, nitrogen, oxygen, and sulfur, or each is separately absent,
provided that at least three of e, f, g, or h are present,
provided that at least one of e, f, g, or h is carbon, and
provided that no two adjacent e, f, g, or h are both oxygen or both sulfur;
R₂, R₃, R₄, and R₅, are each separately selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₂ and R₃, or R₃ and R₄, or R₄ and R₅ taken together, along with the ring carbons to which they are attached, form a five-membered or six-membered cycloalkyl, heterocyclyl or heteroaryl ring, or a six-membered aryl ring moiety;
R₆, R₇, R₈, and R₉, are each separately selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₆ and R₇, or R₇ and R₉, or R₈ and R₉ taken together, along with the ring carbons to which they are attached, form a five-membered or six-membered cycloalkyl, heterocyclyl or heteroaryl ring, or a six-membered aryl ring moiety;
Z is selected from the group consisting of NR₁₁, oxygen, sulfur, and CH₂;
R₁₀ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl optionally substituted aryl, optionally substituted arylalkyl, and perhaloalkyl; and
R₁₁ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl, and optionally substituted arylalkyl;
R₁₂ and R₁₃ are separately selected from the group consiting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₁₂ and R₁₃, taken together, along with the ring carbons to which they are attached, form a five-membered or six-membered cycloalkyl, heterocyclyl or heteroaryl ring, or a six-membered aryl ring moiety;
any bond represented by a dashed and solid line represents a bond selected from the group consisting of a carbon-carbon single bond and a carbon-carbon double bond.

42. The method of claim 41, wherein the compound is N-desmethylclozapine and any clozapine also contacting the D2 receptors is low enough such that the combined N-desmethylclozapine and clozapine contacting the D2 receptors result in a net agonism of the D2 receptors.

43. A method of ameliorating one or more symptoms of a condition associated with a dopamine receptor, comprising:
identifying a subject exhibiting one or more symptoms of a condition associated with a dopamine receptor; and
administering to the subject a therapeutically effective amount of a compound of Formula I, II, or XV: 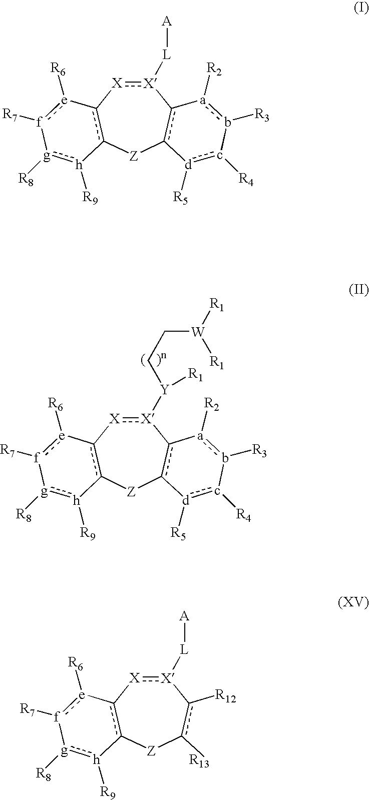
or a pharmaceutically acceptable salt, ester, amide, or prodrug thereof, wherein:
A is selected from the group consisting of 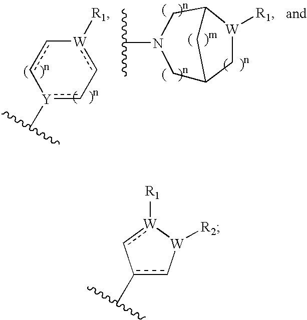
X is nitrogen, CH, or CH₂;
X′ is C or CH, wherein when X′ is C, there is a double bond between X and X′ and wherein when X′ is CH, there is a single bond between X and X′;
each Y is separately selected from the group consisting of nitrogen, oxygen, or CH;
each W is separately selected from the group consisting of nitrogen, CH, oxygen, or sulfur;
each n is separately selected from the group consisting of 0, 1, 2, 3, and 4;
m is selected from the group consisting of 1, 2, and 3;
each R₁ is separately absent or is separately selected from the group consisting of hydrogen, halogen, amine, optionally substituted C₁₋₂₀ alkyl, optionally substituted C₃₋₈ cycloalkyl, optionally substituted C₂₋₂₀ alkenyl, optionally substituted C₂₋₂₀ alkynyl, optionally substituted C₁₋₂₀-alkoxyalkyl, and optionally substituted aryl and arylalkyl;
L is absent or is selected from the group consisting of —NH(CH₂)ₙ— and —(CH₂)ₙ—;
a, b, c, and d are each separately selected from the group consisting of carbon, nitrogen, oxygen, and sulfur, or each is separately absent,
provided that at least three of a, b, c, or d are present,
provided that at least one of a, b, c, or d is carbon, and
provided that no two adjacent a, b, c, or d are both oxygen or both sulfur;
e, f, g, and h are each separately selected from the group consisting of carbon, nitrogen, oxygen, and sulfur, or each is separately absent,
provided that at least three of e, f, g, or h are present,
provided that at least one of e, f, g, or h is carbon, and
provided that no two adjacent e, f, g, or h are both oxygen or both sulfur;
R₂, R₃, R₄, and R₅, are each separately selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₂ and R₃, or R₃ and R₄, or R₄ and R₅ taken together, along with the ring carbons to which they are attached, form a five-membered or six-membered cycloalkyl, heterocyclyl or heteroaryl ring, or a six-membered aryl ring moiety;
R₆, R₇, R₈, and R₉, are each separately selected from the group consisting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₆ and R₇, or R₇ and R₈, or R₈ and R₉ taken together, along with the ring carbons to which they are attached, form a five-membered or six-membered cycloalkyl, heterocyclyl or heteroaryl ring, or a six-membered aryl ring moiety;
Z is selected from the group consisting of NR₁₁, oxygen, sulfur, and CH₂;
R₁₀ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl optionally substituted aryl, optionally substituted arylalkyl, and perhaloalkyl; and
R₁₁ is selected from the group consisting of hydrogen, optionally substituted C₁₋₆ alkyl, optionally substituted C₃₋₈ cycloalkyl, optionally substituted C₂₋₆ alkenyl, optionally substituted C₂₋₆ alkynyl, and optionally substituted arylalkyl;
R₁₂ and R₁₃ are separately selected from the group consiting of hydrogen, halogen, optionally substituted C₁₋₆ alkyl, optionally substituted C₁₋₆ alkyloxy, optionally substituted C₂₋₆ alkenyl, optionally substituted C₂₋₆ alkynyl, optionally substituted C₁₋₆-alkoxyalkyl, optionally substituted C₁₋₆ alkylthio, perhaloalkyl, CN, COR₁₀, CONHR₁₀, NHCONHR₁₀, SO₂NHR₁₀, SO₂R₁₀, OSO₂R₁₀, heteroalkyl, NO₂, NHCOR₁₀,
or R₁₂ and R₁₃, taken together, along with the ring carbons to which they are attached, form a five-membered or six-membered cycloalkyl, heterocyclyl or heteroaryl ring, or a six-membered aryl ring moiety;
any bond represented by a dashed and solid line represents a bond selected from the group consisting of a carbon-carbon single bond and a carbon-carbon double bond.

44. The method of claim 43, wherein the compound is N-desmethylclozapine and the amount of any clozapine administered is low enough such that the combined N-desmethylclozapine and clozapine result in a net agonism at the dopamine receptor.
